SEQUENCE LISTING (22 sequences)

SEQ ID NO: 1 - DNA, 42 bp, Leishmania infantum
ctgaacgctgccgcggagtcaaactcgggtcttgcgtcgaag

SEQ ID NO: 2 - protein, 14 aa, Leishmania infantum
LNAAAESNSGLASK

SEQ ID NO: 3 - DNA, 72 bp, Leishmania infantum
ggcggtggcgagccgtcgggccctttggcgagtgcgatcgtcgacagttttggcagcttcgcgagcttcaag

SEQ ID NO: 4 - protein, 24 aa, Leishmania infantum
GGGEPSGPLASAIVDSFGSFASFK

SEQ ID NO: 5 - DNA, 39 bp, Leishmania infantum
cagaatgatatggtggatatgagctctctatccggcaag

SEQ ID NO: 6 - protein, 13 aa, Leishmania infantum
QNDMVDMSSLSGK

SEQ ID NO: 7 - DNA, 30 bp, Leishmania infantum
atgccatggctttccatcccgttcgagaag

SEQ ID NO: 8 - protein, 10 aa, Leishmania infantum
MPWLSIPFEK

SEQ ID NO: 9 - DNA, 72 bp, Leishmania infantum
cagtacaaggtggaatcgattccgacgctgattggattgaacgcggacacgggtgataccgtgacaacgcgc

SEQ ID NO: 10 - protein, 24 aa, Leishmania infantum
QYKVESIPTLIGLNADTGDTVTTR

SEQ ID NO: 11 - DNA, 63 bp, Leishmania infantum
gtggaatcgattccgacgctgattggattgaacgcggacacgggtgataccgtgacaacgcgc

SEQ ID NO: 12 - protein, 21 aa, Leishmania infantum
VESIPTLIGLNADTGDTVTTR

SEQ ID NO: 13 - DNA, 36 bp, Leishmania infantum
tttgcgaacctgggctttacagaggccgccttcaag

SEQ ID NO: 14 - protein, 12 aa, Leishmania infantum
FANLGFTEAAFK

SEQ ID NO: 15 - DNA, 36 bp, Leishmania infantum
gagcaggtgcagggtgtggacgcgatcatggcacgt

SEQ ID NO: 16 - protein, 12 aa, Leishmania infantum
EQVQGVDAIMAR

SEQ ID NO: 17 - DNA, 588 bp, Leishmania infantum
atgccgttcgctgttcagccgctgccgtacccccacgatgcgctcgcgtcgaagggcatgtcgaaggagcaggtcaccttccaccacgagaagcaccacaaggggtacgccgtgaagctgaacgctgccgcggagtcaaactcgggtcttgcgtcgaagtcgctggtggacatcatcaagtctgagaagggccccgccttcaactgtgcggcgcagattttcaaccacgacttcttctggcgctgcctgtcgccgagaggcggtggcgagccgtcgggccctttggcgagtgcgatcgtcgacagttttggcagcttcgcgagcttcaagaaggaattcacggatgcggccaacggccactttggctccggctgggcgtggctcgtgaaggacaagtcgagtggcaagctgaaggtgctccaaacgcacgacgcgggctgcccgctcacggagcccaacctcgtgccgatcttgacgtgcgatatatgggagcacgcgtactatatcgactacaggaacgaccgcgcgtcctacgtgaacgcattttggaacatggtggactgggatttcgcctctagccagctttaa

SEQ ID NO: 18 - protein, 195 aa, Leishmania infantum
MPFAVQPLPYPHDALASKGMSKEQVTFHHEKHHKGYAVKLNAAAESNSGLASKSLVDIIKSEKGPAFNCAAQIFNHDFFWRCLSPRGGGEPSGPLASAIVDSFGSFASFKKEFTDAANGHFGSGWAWLVKDKSSGKLKVLQTHDAGCPLTEPNLVPILTCDIWEHAYYIDYRNDRASYVNAFWNMVDWDFASSQL

SEQ ID NO: 19 - DNA, 438 bp, Leishmania infantum
atgtccggtgtcagcaagcacttgggggatgtgttgaagctgcagaagcagaatgatatggtggatatgagctctctatccggcaagaccgtgttcttgtacttctcggcaagctggtgcccgccgtgccgtggcttcaccccgaagctggtggagttctacgagaagcatcacaattcaaagaacttcgagatcatacttgcgtcatgggacgaggaggaggaggacttcaatgggtactacagcaagatgccatggctttccatcccgttcgagaagcgcaacgttgtggaggcgctaacgaagcagtacaaggtggaatcgattccgacgctgattggattgaacgcggacacgggtgataccgtgacaacgcgcgcgcgccacgccctgacgcaggaccccgagggcgaacagtttccgtggagagacgagtaa

SEQ ID NO: 20 - protein, 145 aa, Leishmania infantum
MSGVSKHLGDVLKLQKQNDMVDMSSLSGKTVFLYFSASWCPPCRGFTPKLVEFYEKHHNSKNFEIILASWDEEEEDFNGYYSKMPWLSIPFEKRNVVEALTKQYKVESIPTLIGLNADTGDTVTTRARHALTQDPEGEQFPWRDE

SEQ ID NO: 21 - DNA, 375 bp, Leishmania infantum
atgtcgtttgaggatgtcggtgtgggcttcgtgcagcactactacaacttctttgccaaccagcgcgaccagttggccggtatttaccgccccaacacgttgttgacgtggcagaaggagcaggtgcagggtgtggacgcgatcatggcacgttttgcgaacctgggctttacagaggccgccttcaagcaggacagtatcgactgccagccctcaatgagcggcggtgtgatcgtcatcgtgaacggcgaggtgaagttgaggggtgaggatcactcactcaagttcaacgacttcttccacctggcgcaggacaacggtcagtggtacgtgtcaaatcaggtcttcaaccttgtcggcggtggaggtcagtaa

SEQ ID NO: 22 - protein, 124 aa, Leishmania infantum
MSFEDVGVGFVQHYYNFFANQRDQLAGIYRPNTLLTWQKEQVQGVDAIMARFANLGFTEAAFKQDSIDCQPSMSGGVIVIVNGEVKLRGEDHSLKFNDFFHLAQDNGQWYVSNQVFNLVGGGGQ

(19) United States
(12) Patent Application Publication
(10) Pub. No.: US 2013/0236484 A1
(43) Pub. Date: Sep. 12, 2013

(54) LEISHMANIASIS ANTIGEN DETECTION ASSAYS AND VACCINES

(71) Applicant: DETECTOGEN INC., Westborough, MA (US)

(72) Inventors: Antonio Campos-Neto, Westborough, MA (US); Claudia Abeijon, Worcester, MA (US)

(73) Assignee: DETECTOGEN INC., Westborough, MA (US)

(21) Appl. No.: 13/789,699

(22) Filed: Mar. 8, 2013

Related U.S. Application Data
(60) Provisional application No. 61/608,403, filed on Mar. 8, 2012.

(51) Int. Cl. A61K 39/008; C12Q 1/68; G01N 33/68

(52) U.S. Cl. CPC A61K 39/008; G01N 33/6893; C12Q 1/6893
USPC 424/185.1; 530/327; 536/23.7; 435/320.1; 530/387.9; 435/7.1; 435/6.11; 530/325; 530/328; 530/326; 530/350; 435/252.33; 435/7.92; 435/254.2; 435/325; 435/365; 435/362

(57) ABSTRACT

The present invention relates to isolated Visceral leishmaniasis (VL) antigens that are useful in therapeutic and vaccine compositions for stimulating a VL specific immunological response. The identified antigens are also useful in diagnostic assays to determine the presence of active VL in an individual. Combinations of antibodies raised against these antigens are especially useful in detection of VL infections. Accordingly, the present invention includes polypeptide molecules, nucleic acid molecules, vaccine compositions, diagnostic assays, and methods of diagnosis and monitoring treatment related to these VL antigens.

### RELATED APPLICATIONS

This application claims the benefit of U.S. Provisional Application No. 61/608,403, filed Mar. 8, 2012. The entire teachings of the above application are incorporated herein by reference.

### GOVERNMENT SUPPORT

The invention was supported, in whole or in part, by a grant from National Institutes of Health, Grant No. R43AI084259-01. The Government has certain rights in the invention.

### BACKGROUND OF THE INVENTION

Visceral leishmaniasis (VL) occurs on four continents and is endemic in 47 countries with approximately 200 million people at risk of infection and with an annual incidence estimated to be 500,000 cases, according to the World Health Organization. The number of cases is increasing, mostly due to deterioration of social and economic conditions and co-infection with HIV. Most of the approximately 50,000 deaths that occur each year due to VL happen in children. VL is a serious debilitating disease that affects economic productivity and quality of life, and is nearly 100% fatal if not treated promptly. The disease is caused by parasites of the Leishmania donovani complex (L. donovani and L. archibaldi in the Old World, and Leishmania infantum in Southern Europe, Africa, and South America). Notwithstanding the existence of anti-leishmanial drugs, global visceral Leishmaniasis (VL) morbidity and mortality remain high and in many parts of the world are increasing due to co-infection with human immunodeficiency virus. In addition to being a human disease, VL caused by L. infantum is a zoonotic infection as well. Foxes and domestic dogs are major vertebrate reservoirs of the parasite. Canine VL (CVL) is widely distributed in Latin America and Southern Europe. In the USA, the potential for CVL to be a significant problem has been recently highlighted.

These alarming facts have been attributed in part to the inexistence of an efficacious VL vaccine. In addition, the lack of an accurate diagnostic test that can identify active VL versus asymptomatic disease remains a key component of measurements that aim to control this serious disease. Prior to the invention, definitive diagnosis of active VL relied primarily on the direct finding of the Leishmania parasites either in smears or in cultures from spleen or bone marrow aspirates, which are invasive procedures and risky for the patient's health. Importantly, the sensitivity of these tests is in general not high and vary enormously. An alternative to these procedures are a variety of nucleic acid amplification tests. These tests are more sensitive than microscopic examination and parasite culture but they remain restricted to referral hospitals and research centers, despite efforts to simplify them.

Several conventional serological tests have been developed and are available for VL diagnosis. However, the overall principles of these tests, which are often based on the detection of antibody response to parasite antigens, have inherent limitations, particularly for the diagnosis of active VL. First, high serum antibody levels are present in both asymptomatic as well as in active VL. Second, serum anti-leishmania antibodies remain present for several years after cure, which complicates the diagnosis of relapsed VL. Third, a number of individuals from endemic areas with no history of VL have anti-leishmanial antibodies, therefore, complicating the specificity of these tests. Fourth, sensitivity of serological tests in VL/HIV co-infected patients is poor particularly if leishmaniasis occurs post-HIV infection.

With respect to immunity, acquired resistance to leishmaniasis is mediated by T cells. T cell deficient mice rapidly succumb after infection with most species of Leishmania and adoptive transfer of normal T cells confers resistance to the animals. Moreover, patients with AIDS are highly susceptible to VL either as a result of concurrent infection or as a result of reactivation of older sub-clinical infection. Among the T cells CD4+ (Th1) are crucial for resistance and CD8+ T cells seem to participate more in the memory events of the immune response involved in parasite elimination.

Although little success has been achieved in vaccine development to VL, vaccine candidates have been tested in mice and dogs and proven to induce some level of protection. Vaccination using crude antigen preparation obtained from promastigote forms of various species of Leishmania (cutaneous and visceral complexes) have been tested in human clinical trials. The results vary with only modest protection against VL.

The current gold standard for diagnosis of VL is the aspiration of relevant tissues (biopsies) such as spleen, liver, bone marrow, or lymph nodes followed by direct visualization or isolation of the parasite in complex culture medium. Such sample collection is invasive and risky and requires trained personnel to identify the amastigote form of the parasite. Moreover, the sensitivity is variable and unsatisfactory, at about 60-85% (Guerin P. J., Olliaro P., Sundar S., Boelaert M., Croft S. L., Desjeux P., Wasunna M. K., and Bryceson A. D., Visceral leishmaniasis: current status of control, diagnosis, and treatment, and a proposed research and development agenda, Lancet Infect. Dis. 2:494-501 (2002); Ho E. A., Soong T. H., and Li Y., Comparative merits of sternum, spleen and liver punctures in the study of human visceral leishmaniasis, Trans. R. Soc. Trop. Med. Hyg., 41:629-636 (1948); Melby P. C, Recent developments in leishmaniasis, Curr. Opin. Infect. Dis., 15:485-490 (2002); Srivastava P., Dayama A., Mehrotra S., and Sundar S., Diagnosis of visceral leishmaniasis. Trans. R. Soc. Trop. Med. Hyg., 105:1-6 (2011)). Conventional serological tests measure antibody responses to parasite antigens, but because antibodies can persist for years after cure, these tests cannot distinguish active VL from prior exposure (Badaro, R., Lobo I., Munos A., Netto E. M., Modabber F., Campos-Neto A., Coler R. N., and Reed S. G., Immunotherapy for drug-refractory mucosal leishmaniasis, J. Infect. Dis., 194:1151-1159 (2006); De Almeida S. L., Romero H. D., Prata A., Costa R. T., Nascimento E., Carvalho S. F., and Rodrigues V., Immunologic tests in patients after clinical cure of visceral leishmaniasis, Am. J. Trop. Med. Hyg., 75:739-743 (2006); Hailu, A., Pre- and post-treatment antibody levels in visceral leishmaniasis, Trans. R. Soc. Trop. Med. Hyg., 84:673-675 (1990)). In addition, these tests often have low sensitivity in the rapidly growing populations that are co-infected with HIV.

Effective vaccination and accurate early diagnosis of the disease are important to control the disease. A need exists for an alternative approach to conventional serological tests, such as the direct identification of leishmanial antigens in the bodily fluids of humans with active VL. In particular, there is an unmet need for a rapid, sensitive, and specific test to identify people with active VL who need immediate treatment, in order to save their lives and prevent the spread of VL to others in their communities. A further need exits for a vaccine that provides an effective protective immunogenic response to the disease.

### SUMMARY OF THE INVENTION

The present invention relates to isolated polypeptide molecules having an immunogenic portion of the Visceral leishmaniasis (VL) antigen, or a variant of said antigen that differs only in conservative substitutions and/or modifications, wherein the antigen has an amino acid sequence. The sequences can be an amino acid sequence encoded by a nucleic acid molecule having a sequence of SEQ ID NO: 1, 3, 5, 7, 9, 11, 13, 15, 17, 19, 21, or combination thereof; an amino acid sequence encoded by a nucleic acid molecule having the coding region of SEQ ID NO: 1, 3, 5, 7, 9, 11, 13, 15, 17, 19, 21, or combination thereof; an amino acid sequence encoded by a complement of SEQ ID NO: 1, 3, 5, 7, 9, 11, 13, 15, 17, 19, 21, or combination thereof; an amino acid sequence encoded by a nucleic acid molecule that hybridizes to SEQ ID NO: 1, 3, 5, 7, 9, 11, 13, 15, 17, 19, 21, or combination thereof; or an amino acid sequence set forth in SEQ ID NO: 2, 4, 6, 8, 10, 12, 14, 16, 18, 20, 22, or combination thereof. The polypeptide molecule of the present invention, in an aspect, provides an immunogenic specific VL response in a host.

In another embodiment, the isolated polypeptide molecule of the present invention includes an immunogenic portion of a L. infantum or L. donovani antigen, wherein the antigen has one or more of the following amino acid sequences: an amino acid sequence encoded by a nucleic acid having greater than or equal to about 70% identity with SEQ ID NO: 1, 3, 5, 7, 9, 11, 13, 15, 17, 19, 21, or combination thereof; an amino acid sequence encoded by a nucleic acid molecule having greater than or equal to about 70% identity with coding region of SEQ ID NO: 1, 3, 5, 7, 9, 11, 13, 15, 17, 19, 21, or combination thereof; a an amino acid sequence encoded by a complement having greater than or equal to about 70% identity with of SEQ ID NO: 1, 3, 5, 7, 9, 11, 13, 15, 17, 19, 21, or combination thereof; an amino acid sequence encoded by a nucleic acid molecule having greater than or equal to about 70% identity with a molecule that hybridizes to SEQ ID NO: 1, 3, 5, 7, 9, 11, 13, 15, 17, 19, 21, or combination thereof; or an amino acid sequence having greater than or equal to about 70% similarity to a sequence set forth in SEQ ID NO: 2, 4, 6, 8, 10, 12, 14, 16, 18, 20, 22, or combination thereof. The isolated polypeptide can have, e.g., an 80%, 90% or greater identity or similarity, respectively, with said sequences.

The present invention also pertains to an isolated nucleic acid molecule that encodes a polypeptide molecule that has an immunogenic portion of a VL antigen, or a variant of said antigen that differs only in conservative substitutions and/or modifications. The antigen is encoded by a nucleic acid molecule having a sequence of SEQ ID NO: 1, 3, 5, 7, 9, 11, 13, 15, 17, 19, 21 or combination thereof; the coding region of SEQ ID NO:1, 3, 5, 7, 9, 11, 13, 15, 17, 19, 21, or combination thereof; a complement of SEQ ID NO: 1, 3, 5, 7, 9, 11, 13, 15, 17, 19, 21, or combination thereof; that hybridizes to SEQ ID NO:1, 3, 5, 7, 9, 11, 13, 15, 17, 19, 21, or combination thereof; or that encodes SEQ ID NO: 2, 4, 6, 8, 10, 12, 14, 16, 18, 20, 22, or combination thereof. In an embodiment the isolated nucleic acid molecule can be an RNA molecule.

Yet another embodiment of the present invention relates to an isolated nucleic acid molecule that encodes a polypeptide molecule that has an immunogenic portion of a L. infantum or L. donovani antigen, wherein the antigen is encoded by a nucleic acid molecule having greater than or equal to about 70% identity with one or more of the following sequences: SEQ ID NO: 1, 3, 5, 7, 9, 11, 13, 15, 17, 19, 21, or combination thereof; the coding region of SEQ ID NO: 1 1, 3, 5, 7, 9, 11, 13, 15, 17, 19, 21, or combination thereof; a complement of SEQ ID NO: 1, 3, 5, 7, 9, 11, 13, 15, 17, 19, 21, or combination thereof; that hybridizes to SEQ ID NO: 1, 3, 5, 7, 9, 11, 13, 15, 17, 19, 21, or combination thereof; or that encodes SEQ ID NO: 2, 4, 6, 8, 10, 12, 14, 16, 18, 20, 22, or combination thereof. The isolated nucleic acid molecule can have, for example, an 80%, 90% or greater identity or similarity, respectively, with said sequences.

The present invention further includes vectors, plasmids, or cells having the nucleic acid molecule described herein or can express the polypeptide molecule described herein. The invention also includes one or more antibodies that bind to a polypeptide described herein. Fusion proteins are also encompassed by the present invention and have one or more of the polypeptides described herein, and can optionally further include a known L. infantum or L. donovani antigen or an L. infantum or L. donovani antigen presented on a MHC Class-2 molecule.

The present invention also embodies a method for stimulating a specific immunogenic VL response in an individual, or preventing or reducing the severity of VL disease. The method includes the step of administering an amount of one or more of the polypeptide or nucleic acid molecules described herein (e.g., a vaccine) with or without a carrier.

Treatment of the VL disease can also be monitored using the methods of the present invention. The methods include the step of detecting the level of one or more of the polypeptide molecules described herein in a sample from the individual; and comparing the level of the one or more molecules with a standard. The level of molecules that is higher than the standard indicates ineffective treatment, and a level that is less than or equal to the standard indicates effective treatment. The present invention also includes detecting the level of the polypeptide molecules in a sample from the individual at more than one time point; and comparing the level of the polypeptide molecules at the one or more time point. An increase in the level of the molecules indicates ineffective treatment, and decrease or no change in the level indicates effective treatment.

Additionally, methods of diagnosing VL disease in an individual relate to the present invention. The method includes the steps of detecting the presence or absence of one or more of the polypeptide molecules described herein. The presence of the one or more polypeptide molecules indicates the presence of the VL disease and the absence of the one or more polypeptide molecules indicates the absence of the disease.

The present invention further includes methods of distinguishing between VL disease, and asymptomatic VL or immunity to the VL disease in an individual. The steps of the method involve detecting the presence or absence of one or more of the polypeptide molecules described herein; and measuring the stimulation of a VL specific immune response in the individual. Measuring the stimulation of a VL specific immune response includes measuring cell proliferation, interferon-γ levels, or a combination thereof, in a sample from the individual.

Methods for detecting L. infantum or L. donovani infection in a biological sample are included in the present invention and involve assessing the presence of one or more of the polypeptide molecules described herein in the sample (e.g., urine). The presence of the molecule indicates the presence of L. infantum or L. donovani infection; and the absence of the molecule indicates the absence of L. infantum or L. donovani infection. Assessment can include contacting the sample with an antibody that binds with the polypeptide molecule, sufficiently to allow formation of a complex between the sample and the antibody, to thereby form an antigen-antibody complex; and detecting the antigen-antibody complex. The presence of the complex indicates the presence of L. infantum or L. donovani infection; and the absence of a complex indicates the absence of L. infantum or L. donovani infection. The antibody can be detectably labeled. The method further includes contacting the sample with a second antibody specific to said antigen or said antigen-antibody complex.

Also included in the present invention are methods for detecting L. infantum or L. donovani infection in a biological sample, in which more than one antibody is used. The presence of one or more of the polypeptide molecules described herein in a sample such as urine is assessed by the use of a combination of antibodies. Assessment can include contacting the sample with an antibody mixture that includes at least one component that binds with the polypeptide molecule, sufficiently to allow formation of a complex between the sample and a component of the antibody mixture, to thereby form an antigen-antibody complex; and detecting the antigen-antibody complex. The presence of the complex, similarly to the method of using an antibody, indicates the presence of L. infantum or L. donovani infection; and the absence of a complex indicates the absence of L. infantum or L. donovani infection. The antibody can be detectably labeled. The method further includes contacting the sample with a second antibody specific to said antigen or said antigen-antibody complex. The number of antibodies used can be two, three, four, or more. Each antibody can be against a different protein or a different peptide. The present invention includes the use of antibodies raised against the same antigen, for example when they recognize different epitopes of the antigen.

Detection of L. infantum or L. donovani infection in a biological sample can also be done by contacting the sample with at least two oligonucleotide primers in a polymerase chain reaction, wherein at least one of the oligonucleotide primers is specific for one or more of the isolated nucleic acid molecule described herein, sufficiently to allow amplification of the primers; and detecting in the sample the amplified nucleic acid sequence. The presence the amplified nucleic acid sequence indicates L. infantum or L. donovani infection, and the absence of the amplified nucleic acid sequence indicates an absence of L. infantum or L. donovani infection. Similarly, detection for L. infantum or L. donovani infection in a biological sample can also be done by contacting the sample with one or more oligonucleotide probes specific for the nucleic acid molecule described herein under high stringency conditions, sufficiently to allow hybridization between the sample and the probe; and detecting the nucleic acid molecule that hybridizes to the oligonucleotide probe in the sample. The presence of hybridization of the probe indicates L. infantum or L. donovani infection, and the absence of hybridization indicates an absence of L. infantum or L. donovani infection.

The present invention also relates to compositions (e.g., a pharmaceutical composition) that include the polypeptide sequence described herein and a physiologically acceptable carrier. The composition can further include an immune response enhancer (e.g., an adjuvant, for example, MPL or QS21, or another VL antigen). The immune response enhancer can also be an immunostimulatory cytokine or chemokine. The composition can also be a vaccine composition.

Yet another embodiment of the present invention includes kits. The kits for diagnosing the presence or absence of L. infantum or L. donovani infection in a person include one or more reagents for detecting the polypeptide molecule described herein; one or more nucleic acid molecules having a sequence of SEQ ID NO:1, 3, 5, 7, 9, 11, 13, 15, 17, 19, 21, or combination thereof; the complements of said sequences, or nucleic acid sequences that hybridize to a sequence recited in SEQ ID NO:1, 3, 5, 7, 9, 11, 13, 15, 17, 19, 21, or combination. The kits can also include a detection reagent.

Advantages of the present invention include new methods for preventing or reducing the severity of the VL disease by providing an effective vaccine composition. The assays of the present invention allow simple and easy to administer urine tests to quickly and efficiently distinguish between patients having active VL and those who do not. New vaccine compositions and more effective diagnostic assays will assist in reducing the present worldwide VL problem.

### BRIEF DESCRIPTION OF THE DRAWINGS

FIG. 1, panel A is a photograph showing the pattern of urine protein bands that were cut from polyacrylamide gels for mass spectrometry analyses, and FIG. 1, panels B-C are photographs showing purification and characterization of the recombinant Li-isd1, Li-txn1, and Li-ntf2. For panel A, urine was from a patient with visceral leishmaniasis (VL). Urine was concentrated 10× using a Centricon P3 (3-kDa cutoff), mixed with sample buffer, and then submitted to SDS-PAGE (polyacrylamide gel with a 4 to 20% gradient), followed by Coomassie staining Bands ranging from ˜10 kDa to ˜75 kDa were cut from the gel and submitted to mass spectroscopy analyses. Numbered boxes on the right side of the gel indicate the individual bands that were cut from the gel. Lane 1, 10× concentrated urine from VL patient; MM, molecular markers. Numbers on the left of the gel are the molecular masses of the markers (A). Recombinant proteins containing six His-tag amino terminal residues were expressed in E. coli BL21(DE3)pLysS, followed by purification by affinity chromatography using Ni-NTA agarose matrix. Purity was evaluated by SDS-PAGE (4-20% gradient polyacrylamide gel) and Coomassie blue staining (B). Lane 1, Li-isd1; lane 2, Li-txn1; lane 3, Li-ntf2. (C) Characterization of the proteins was done by Western blot analysis. 50 ng of purified recombinant proteins (lanes R) and 2 μg of crude antigenic preparation of promastigote forms of L. infantum (lanes N, for native proteins) were submitted to electrophoresis under reducing conditions in 4-20% gradient polyacrylamide gel and transferred to PVDF membrane. Proteins were identified using specific rabbit anti-recombinant protein antisera. Reactivity was detected with goat anti-rabbit IgG labeled with horseradish peroxidase and a luminol-based analysis system, followed by autoradiography. Numbers on the left side indicate the molecular masses of the markers.

FIG. 2 provides a series of graphs showing the recognition of the recombinant proteins Li-isd1, Li-txn1, and Li-ntf2 by sera from patients with VL. Recognition of the antigens was determined by ELISA. Microtiter plates (96-well) were coated with 100 ng of antigen/well, blocked, and incubated with 1/50 dilution of the sera from VL patients and healthy control subjects. Antibody reactivity was measured using peroxidase-labeled goat anti-human IgG. Reactions were developed after addition of the substrate (H₂O₂) and the chromophore TMB. Results are expressed as ODs read at 450 nm. Symbols represent the results obtained for urine specimens of seven individual patients with VL (A) and five normal control subjects (B).

FIG. 3, panels A-C, include graphs showing the determination of the limit of detection (LOD) of capture ELISAs assembled for the proteins Li-isd1, Li-txn1, and Li-ntf2 spiked in urine specimens of normal healthy subjects. Capture antibodies at previously determined concentrations were used to coat the ELISA plates. The following antibody concentrations were used: antigen affinity-purified IgY anti-Li-isd1, 100 ng/well (A); antigen affinity-purified rabbit anti-Li-txn1 antibodies, 875 ng/well (B); and purified rabbit IgG anti-Li-nom antibody (2,000 ng/well) (C). Wells were then incubated with various concentrations of the antigen diluted either in saline plus 1% BSA or in urine from normal healthy subjects, followed by incubation with biotin-labeled anti-antigen secondary antibody. Reactions were developed after addition of peroxidase-labeled streptavidin, substrate (H₂O₂), and the chromophore TMB. Results are expressed as ODs read at 450 nm.

FIG. 4, panels A-C, is a series of bar graphs showing antigen detection assays for the identification of the proteins Li-isd1, Li-txn1, and Li-ntf2 in urine of VL patients and controls. Urine specimens were from VL patients and normal, healthy control subjects. Assays were carried out using capture ELISAs assembled as described in the legend to FIG. 3. (A) Anti-Li-isd1. (B) Anti-Li-txn1. (C) Anti-Li-nom. Samples from VL patients (n=19) were from Teresina, PI, Brazil. Control samples were from healthy individuals from countries where VL is endemic who were living in the United States. Dashed lines represent the cutoff values, which were calculated using the average of the ODs obtained from the urine specimens from 16 normal, healthy control subjects plus 3 SDs. Note that, compiling the results obtained for the three proteins, the antigen detection assay was positive in 17/19 VL patients. These are representative results of at least three experiments performed at different times with the same urine samples and the same capture ELISA.

FIG. 5, panels A-C, is a series of bar graphs showing the specificity of capture ELISA for the Li-isd1, Li-txn1, and Li-ntf2. Urine specimens were from patients with cutaneous leishmaniasis (CL) n=10, Chagas disease (CD) n=8, schistosomiasis (SC) n=14, tuberculosis (TB) n=10, from normal, healthy control subjects (HLT) n=16; or from patients with VL (n=19). Assays were carried out using capture ELISAs assembled as described in the legend to FIG. 3. (A) Anti-Li-isd1. (B) Anti-Li-txn1. (C) Anti-Li-nom. Dashed lines represent the cutoff values, which were calculated using the average of the ODs obtained from the urine specimens from 16 normal, healthy control subjects plus 3 SDs. Note that, no cross-reaction was observed with any of the 42 urine specimens with diseases other than VL. The box-and-whiskers diagram was used to highlight the differences and dispersions (the black components of the bars indicate skewness) of the ODs obtained for the urine specimens from VL patients compared to the ODs obtained for CL, CD, SC, or TB and healthy control subjects.

FIG. 6 is a schematic representation of antigen discovery strategy of L. infantum chagasi proteins produced in vivo and excreted in urine of patients with active VL.

FIG. 7 is a schematic showing L. infantum chagasi peptide sequences discovered in urine of VL patients. Peptide sequences (SEQ ID NO: 2, 4, 6, 8, 10, 12, 14, and 16) and their positioning within the peptide donor protein sequences (SEQ ID NO: 18, 20, and 22) are highlighted in bold/underline. Note that several different peptides spanning at different sequence positions of the donor proteins were identified, which unambiguously define them as truly L. infantum chagasi peptides/proteins.

FIG. 8, panels A-B, is a set of photographs showing the purification and characterization of the recombinant Li-ntf2. Purity of the protein was evaluated by SDS-PAGE (4-20% gradient polyacrylamide gel) and Coomassie blue staining (A). Characterization was carried out by Western blot analysis (B) using crude antigenic preparations of L. infantum promastigotes (lane 1) and the purified recombinant Li-ntf2 (lane 2). Proteins were identified using specific rabbit anti-recombinant Li-ntf2 antiserum. Reactivity was detected with goat anti-rabbit IgG labeled with horseradish peroxidase, followed by autoradiography. Numbers on the left side indicate the molecular weights of the markers.

FIG. 9 shows line graphs of the isotype specific antibody response of mice immunized with purified recombinant Li-ntf2 formulated with the adjuvant MPLA-SE. Ten mice were immunized three times (three weeks apart) with saline plus the adjuvant BpMPLA-SE (A), or with the Li-ntf2 in saline (B), or with Li-ntf2 mixed BpMPLA-SE (C). Animals were bled 10 days after the last immunization, and antibody responses of IgG1 and IgG2a isotypes were measured by ELISA using biotinylated isotype rat mAbs specific for mouse IgG isotypes. The curves represent the responses of each individual mouse.

FIG. 10 shows graphs of cytokine response of spleen cells from mice immunized with purified recombinant Li-ntf2 formulated with the adjuvant BpMPLA-SE. BALB/c mice (three) were immunized as described in FIG. 9. Spleen cells were harvested 10 days after the last immunization either with saline+BpMPLA-SE or with purified recombinant Li-ntf2+BpMPLA-SE. Cells were cultured for 72 h in the presence of medium only, or with 5 μg/mL of the recombinant protein, or with 5 μg/mL of ConA. Culture supernatants were collected and assayed for the presence of IFN-γ and IL-4 by sandwich ELISA.

FIG. 11, panels A-C, shows graphs of the phenotype of CD4+ T cell response of mice immunized with purified recombinant Li-nom formulated with the adjuvant BpMPLA-SE. Three mice were immunized as described in FIG. 9. Spleen cells were harvested 10 days after the last immunization either with saline+BpMPLA-SE or with purified recombinant Li-ntf2+BpMPLA-SE. Production of IFN-γ (Upper panel) and IL-4 (Lower panel) by CD4+ T cells was determined by intra-cellular cytokine staining and analyzed by flow cytometry (A). Dot plots represent the responses of individual mouse in each group after stimulation with Li-ntf2; (B) Percentage double positive CD4+/IFN-γ+ T cells (upper panel) or CD4+/IL-4+ T cells (lower panel) stimulated with the Li-ntf2 (average of responses of cells from three mice per group); and (C) Percentage double-positive CD4+/IFN-α+ T cells (upper panel) or CD4+/IL-4+ T cells (lower panel) stimulated with the polyclonal activators PMA+Ionomicin (average of responses of cells from three mice per group). Note the abundance of CD4+/IFN-γ+ double-positive cells and the scarce presence of CD4+/IL4+ double-positive cells in cultures obtained from mice immunized with Li-ntf2+ BpMPLA-SE and re-stimulated in vitro with Li-ntf2. Cells from both groups of immunized mice produced abundant quantities of both IFN-γ and IL-4 when stimulated with PMA+Ionomicin. Little or no cytokine-producing cells were observed in the cell cultures from animals immunized with saline+BpMPLA-SE and equally re-stimulated in vitro with the antigen. Similarly, no cytokine-producing cells were detected in any culture stimulated with medium only (not shown).

FIG. 12 is a bar graph showing the vaccination of mice against VL with purified recombinant Li-ntf2 formulated with the adjuvant BpMPLA-SE. BALB/c mice (7 animals per group) were immunized three times as described in FIG. 9. As control, mice (n=7) were immunized with saline plus BpMPLA-SE. 30 days after the last immunization, the animals were challenged i.v. with 10×10⁶ promastigote forms (stationary phase of growth) of L. infantum chagasi. Parasite burden in the animals' spleens was determined 40 days later by limiting dilution assay. Bars represent SEM of the results given by 7 mice per group. Student's t test was used to compare the average of parasite burden observed for animals immunized with Li-nft2+BpMPLA-SE with that of animals immunized with Saline+BpMPLA-SE. P value was <0.005.

FIGS. 13A-C are schematics showing the sequence of the nucleic acid molecules (SEQ ID NO: 1, 3, 5, 7, 9, 11, 13, 15, 17, 19, and 21) that encode the polypeptide molecules (SEQ ID NO:2, 4, 6, 8, 10, 12, 14, 16, 18, 20, and 22) found in the urine of patients with VL.

FIG. 14 is a drawing showing an example of an immunochromatographic assay for the detection of VL antigens of the present invention in a sample.

FIG. 15, panels A-D, is a series of bar graphs showing antigen detection capture ELISAs for the identification of the proteins Li-isd1, Li-txn1, and Li-ntf2 in urine of VL patients and controls. Urine specimens were from VL patients and normal, healthy control subjects. The following predetermined capture antibody concentrations were used to coat the ELISA plates: antigen affinity-purified IgY anti-Li-isd1, 100 ng/well; antigen affinity-purified rabbit anti-Li-txn1 antibodies, 875 ng/well; and purified rabbit IgG anti-Li-ntf2 antibody (2,000 ng/well), or the combination of the three antibodies/well. Samples from VL patients (n=20) were from Teresina, PI, Brazil. Control samples were from healthy individuals from countries where VL is endemic who were living in the United States. Dashed lines represent the cutoff values calculated as described in the text. These are representative results of at least three experiments performed at different times with the same urine samples and same capture ELISA.

FIG. 16 is a bar graph showing specificity of the capture ELISA assembled with a combination of antibodies specific to Li-isd1, Li-txn1, and Li-ntf2 antigens. Urine specimens were from patients with cutaneous leishmaniasis (CL) (n=10), Chagas' disease (CD) (n=8), schistosomiasis (SC) (n=14), or tuberculosis (TB) (n=10). Assay was performed using capture ELISAs assembled with all of the three antibodies in a single well as described in the legend for FIG. 14. Dashed line represents the cutoff values, which was calculated using the average of the ODs obtained from the urine specimens from 20 normal, healthy control subjects plus 3 SDs.

### DETAILED DESCRIPTION OF THE INVENTION

A description of preferred embodiments of the invention follows.

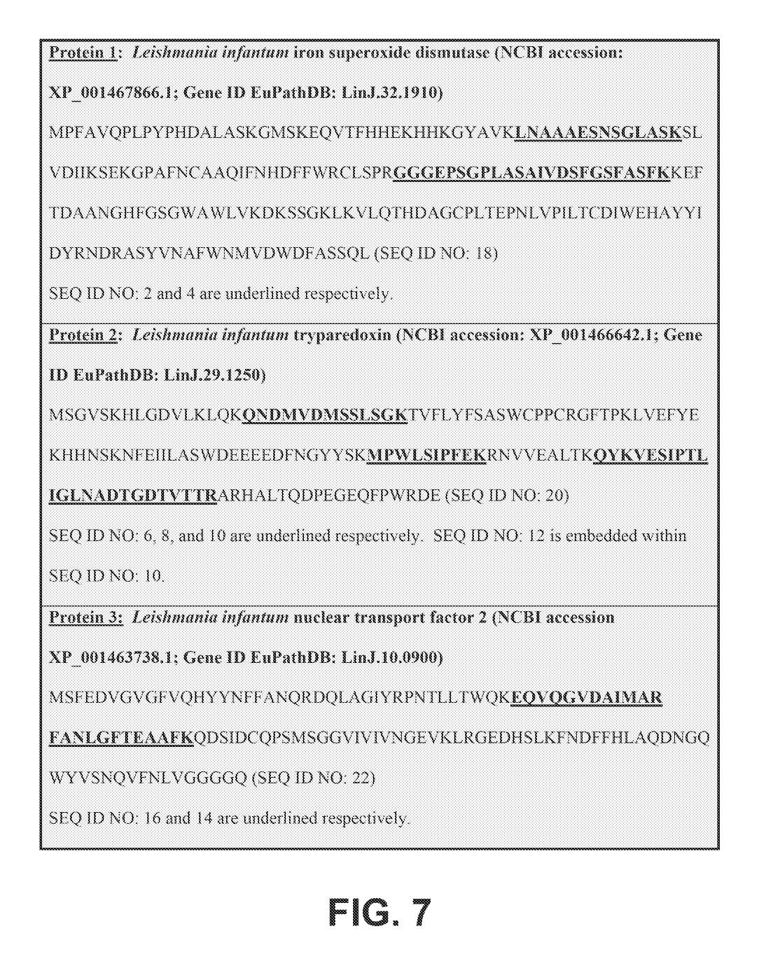

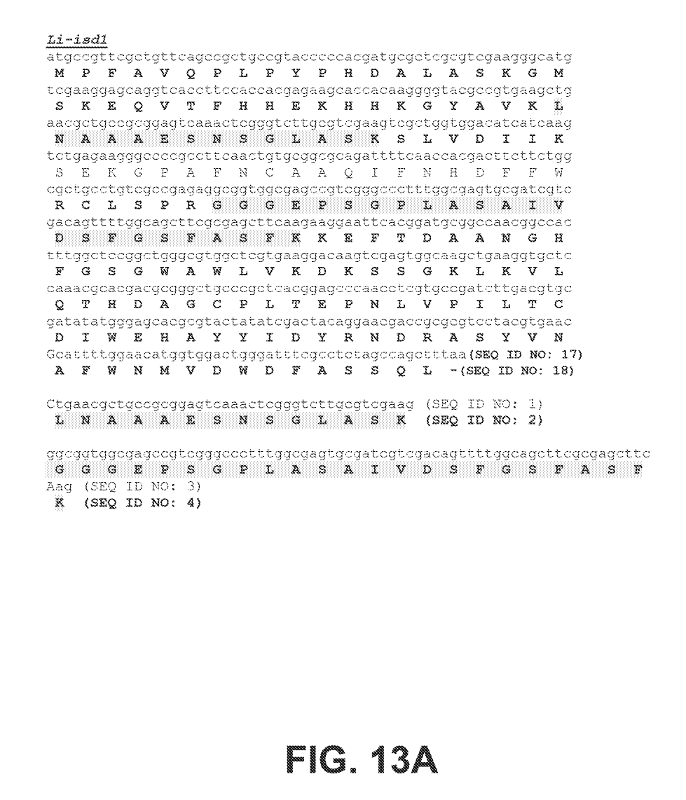

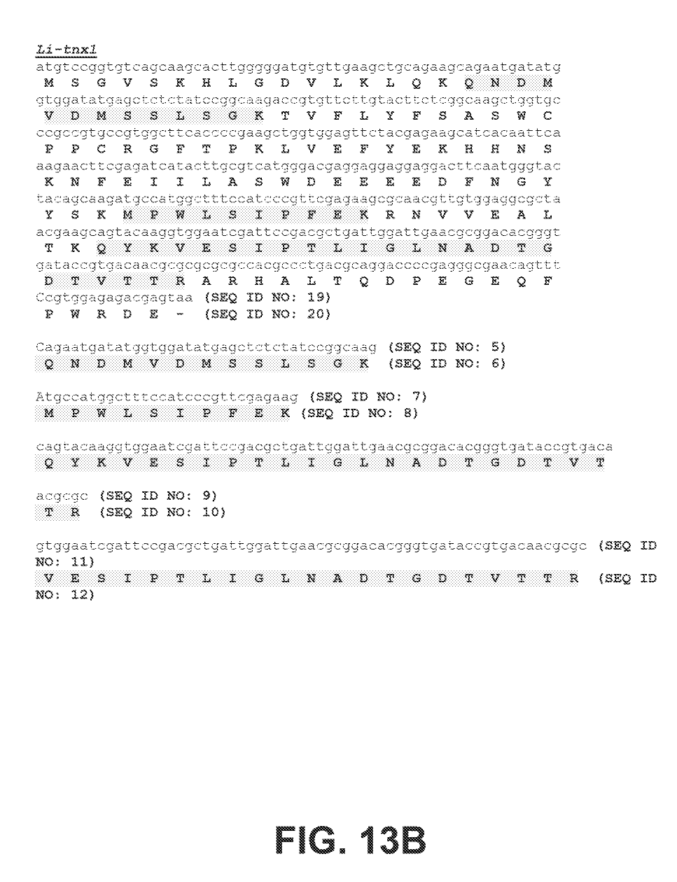

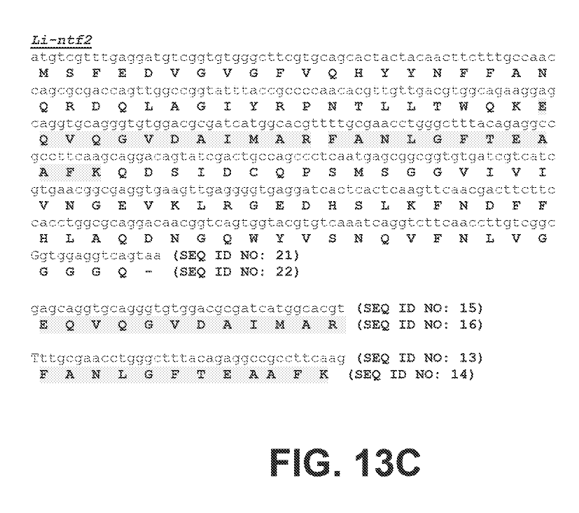

The present invention relates to vaccine compositions and diagnostic assays for the Visceral leishmaniasis (VL) disease. The present invention is based, in part, on the discovery of certain VL antigenic peptides that allow for the identification of a VL peptide present in the urine of people infected with active VL. The present invention is also based on a second discovery that these peptides provide an immunogenic effect when administered as a vaccine. Hence, the present invention, in part, relates to the specific antigenic VL polypeptide sequences discovered, which are shown in FIGS. 7 and 13A-C namely, SEQ ID NOs 2, 4, 6, 8, 10, 12, 14, 16, 18, 20, 22, and combinations thereof. SEQ ID NOs: 2, 6, 10, 14, and 16 were isolated by obtaining a total of 25 urine samples from patients with VL. SEQ ID NOs: 1, 3, 5, 7, 9, 11, 13, 15, 17, 19, and 21, the nucleic acid sequences that encode these identified polypeptides, are also shown in FIGS. 13A-C.

To determine antigens useful in the detection of active VL, samples were collected from patients diagnosed with VL based on the following criteria: a clinical course consistent with VL (e.g., fever, anemia, hepatosplenomegaly), and confirmatory laboratory findings (identification of Leishmania in bone marrow aspirates). Individual urine samples were analyzed by mass spectrometry generating a total of approximately 400 peptide sequences. Antibodies were made, and western blot and ELISA analysis performed to determine which antigens are useful in detecting active VL in the urine of patients. See Exemplification, Example 1. Most sequences of the identified peptides had identical sequence homologies with that of human proteins. However, eight peptide sequences, SEQ ID NO: 2, 4, 6, 8, 10, 12, 14, 16 (and accordingly 18, 20, and 22) that had no known homologies with human proteins had identical sequence homologies with the deduced sequences of three different L. infantum proteins, Iron superoxide dismutase (Li-isd1), Tryparedoxin (Li-txn1), and Nuclear transport factor 2 (Li-ntf2).

The genes coding for the identified L. infantum proteins are highly conserved among the organisms of the L. donovani complex. Therefore, the antibodies raised against the L. infantum recombinant proteins recognize equally well the proteins produced by L. donovani thus suggesting that that the proposed test will be equally sensitive to detect VL caused by either L. donovani or L. infantum.

Polypeptides useful in vaccines were also determined. A total of seven urine samples were evaluated and collected from patients diagnosed with VL based on the following criteria: a clinical course consistent with VL (e.g., fever, anemia, hepatosplenomegaly), and confirmatory laboratory findings (identification of Leishmania in bone marrow aspirates). The samples were analyzed with SDS-PAGE and mass spectroscopy methods. The specific fragmentation pattern was computer-searched against predicted tryptic peptides from all known proteins from genome sequencing projects of human and Leishmania. The peptides were scored using the methodology described in Example 2, and certain peptides were identified. Challenge experiments were performed in mice and T cell immunogenicity assays were carried out. Immunization with polypeptides of the present invention caused significant reduction in the parasite burden as compared to animals not immunized with the inventive polypeptides. See Exemplification, Example 2. In an embodiment, the vaccine composition includes the Li-ntf2 (SEQ ID NO: 22) or portions thereof (e.g., SEQ ID NO: 14 and 16) and optionally other polypeptides of the present invention or other immune stimulating compounds (e.g., monophosphoryl lipid A (MPLA)), as further described herein.

The present invention also relates to a combination of antibodies that can be used simultaneously, for example in a capture ELISA. The mixture of antibodies, for example a mixture of three antibodies, can contain antibodies raised against the full-length or partial-length polypeptides of SEQ ID NOs: 18, 20, and 22.

Accordingly, the present invention relates to these sequences, SEQ ID NO: 1-22, that have been identified as being useful in eliciting a protective immune response against VL, and for diagnostic assays for identifying persons with active VL.

### VL Diagnostic Assay, Antibodies and Methods of Assessment

Method for assessing the presence or absence of the antigenic VL polypeptides described herein, in a sample, are encompassed by the present invention. Suitable assays include immunological methods, such as radioimmunoassay, enzyme-linked immunosorbent assays (ELISA), chemiluminescence assays, and rapid immunochromatographic assays. Any method known now or developed later can be used for measuring antigenic VL polypeptides.

Antibodies reactive with any one of the antigenic VL polypeptides, namely, SEQ ID NOs: 2, 4, 6, 8, 10, 12, 14, 16, 18, 20, 22, or a combination thereof, or portions thereof can be used. In a preferred embodiment, the antibodies specifically bind with antigenic VL polypeptides or a portion thereof. The antibodies can be polyclonal or monoclonal, and the term antibody is intended to encompass polyclonal and monoclonal antibodies, and functional fragments thereof. The terms polyclonal and monoclonal refer to the degree of homogeneity of an antibody preparation, and are not intended to be limited to particular methods of production.

In several of the preferred embodiments, immunological techniques detect the presence, absence of levels of antigenic VL polypeptides described herein by means of an anti-VL antibody (i.e., one or more antibodies). The term “anti-VL antibody” includes monoclonal and/or polyclonal antibodies, and mixtures or cocktails thereof, and refers to antibodies specific to polypeptides having a sequence set forth in SEQ ID Nos: 2, 4, 6, 8, 10, 12, 14, 16, 18, 20, 22, or combination thereof, or portions thereof.

Anti-VL antibodies can be raised against appropriate immunogens, such as isolated and/or recombinant antigenic VL polypeptides described herein, analogs or portion thereof (including synthetic molecules, such as synthetic peptides). In one embodiment, antibodies are raised against an isolated and/or recombinant antigenic VL polypeptides described herein or portion thereof (e.g., a peptide) or against a host cell which expresses recombinant antigenic VL polypeptides. In addition, cells expressing recombinant antigenic VL polypeptides described herein, such as transfected cells, can be used as immunogens or in a screen for antibody which binds receptor.

Any suitable technique can prepare the immunizing antigen and produce polyclonal or monoclonal antibodies. The art contains a variety of these methods (see e.g., Kohler et al., Nature, 256: 495-497 (1975) and Eur. J. Immunol. 6: 511-519 (1976); Milstein et al., Nature 266: 550-552 (1977); Koprowski et al., U.S. Pat. No. 4,172,124; Harlow, E. and D. Lane, 1988, Antibodies: A Laboratory Manual, (Cold Spring Harbor Laboratory: Cold Spring Harbor, N.Y.); Current Protocols In Molecular Biology, Vol. 2 (Supplement 27, Summer '94), Ausubel, F. M. et al., Eds., (John Wiley & Sons: New York, N.Y.), Chapter 11, (1991)). Generally, fusing a suitable immortal or myeloma cell line, such as SP2/0, with antibody producing cells can produce a hybridoma. Animals immunized with the antigen of interest provide the antibody producing cell, preferably cells from the spleen or lymph nodes. Selective culture conditions isolate antibody producing hybridoma cells while limiting dilution techniques produce them. Researchers can use suitable assays such as ELISA to select antibody producing cells with the desired specificity.

Other suitable methods can produce or isolate antibodies of the requisite specificity. Examples of other methods include selecting recombinant antibody from a library or relying upon immunization of transgenic animals such as mice.

The present invention includes assays to determine if a person is infected with active VL, as compared with person who does not have active VL (e.g., had VL but has been treated, no VL infection, or has been immunized against VL infection). Latent or asymptomatic VL occurs when a person has been infected with L. infantum, but the parasite is dormant or inactive. Active VL infection refers to a person infected with L. infantum and the parasite is acutely affecting portions of the body, including the skin, mucous membranes, abdominal organs such as the spleen or liver, and other tissues. The present invention, based on the discovery that certain VL antigens are found in the urine of patients with active VL, includes assays for determining the absence or presence of active VL infection.

According to the method, an assay can determine the presence, absence, or level of antigenic VL polypeptides in a biological sample. Such an assay includes combining the sample to be tested with an antibody having specificity for antigenic VL polypeptides described herein, under conditions suitable for formation of a complex between antibody and antigenic VL polypeptides, and detecting or measuring (directly or indirectly) the formation of a complex. The sample can be obtained directly or indirectly (e.g., provided by a healthcare provider), and can be prepared by a method suitable for the particular sample (e.g., urine, sputum, cerebral spinal fluid, whole blood, platelet rich plasma, platelet poor plasma, serum) and assay format selected. Methods of combining sample and antibody, and methods of detecting complex formation are also selected to be compatible with the assay format.

Suitable labels can be detected directly, such as radioactive, fluorescent or chemiluminescent labels. They can also be indirectly detected using labels such as enzyme labels and other antigenic or specific binding partners like biotin. Examples of such labels include fluorescent labels such as fluorescein, rhodamine, chemiluminescent labels such as luciferase, radioisotope labels such as ³²P, ¹²⁵I, ¹³¹I, enzyme labels such as horseradish peroxidase, and alkaline phosphatase, galactosidase, biotin, avidin, spin labels, and the like. The detection of antibodies in a complex can also be done immunologically with a second antibody, which is then detected (e.g., by means of a label). Conventional methods or other suitable methods can directly or indirectly label an antibody. Labeled primary and secondary antibodies can be obtained commercially or prepared using methods know to one of skill in the art (see Harlow, E. and D. Lane, 1988, Antibodies: A Laboratory Manual, (Cold Spring Harbor Laboratory: Cold Spring Harbor, N.Y.).

In a preferred embodiment, the presence, absence, or level of antigenic VL polypeptides in a sample is determined using an ELISA assay, a sandwich ELISA assay, or immunochromatographic assay. For detection of antigenic VL polypeptides in a suitable sample, a sample (e.g., urine) is collected. Samples can be processed as known in the art. The assay further includes combining a suitable sample with a composition having an anti-VL polypeptide antibody as detector (e.g., biotinylated anti-VL polypeptides MAb and HRP-streptavidin, or HRP-conjugated anti-VL polypeptides Mab), and a solid support, such as a microtiter plate or dipstick, having an anti-VL polypeptide capture antibody bound (directly or indirectly) thereto. The detector antibody binds to a different antigenic VL polypeptide epitope from that recognized by the capture antibody, under conditions suitable for the formation of the complex. The assay then involves determining the formation of complex in the samples. The presence of one or more of the antigenic VL polypeptide in a sample of an individual indicates the presence of active VL infection, whereas the absence of a VL polypeptide indicates that the patient does not have active VL infection.

The ELISA assay can also be used in which two or more antibodies (e.g., 2, 3, 4, 5, 6, 7, 8, 9, 10, etc.) are used in combination. In one embodiment, three antibodies are used together. The first, second, and third of these three antibodies can be raised against the polypeptides of SEQ ID NOs: 18, 20, and 22, respectively. In another embodiment, the antibodies raised against any combination of two or more of SEQ ID NOs: 2, 4, 6, 8, 10, 12, 14, 16, 18, 20, 22 are used together. It has been discovered that usage of more than one antibody enhances the detective power of the assay, as demonstrated in Example 3. In Example 3, usage of three antibodies in concert was able to detect correctly all 20 out of 20 samples, in contrast to the lower success rate of the assays using a single antibody. It has also been discovered that usage of multiple antibodies does not only have the additive advantage of more quickly getting the combined result of the positives from the individual assays using a single antibody, but also the unexpected advantage of being able to detect positives that none of the used single antibodies could detect alone.

The combined antibody assay, in certain embodiments, uses at least three antibodies, each of which is raised against polypeptides from the following groups: one or more from SEQ ID NOs: 2 and 4; one or more from SEQ ID NOs: 6, 8, 10, 12; and one or more from SEQ ID NOs: 14 and 16. In some embodiments, antibodies are raised against different sequences that are portions of SEQ ID NOs: 18, 20, and/or 22. Combinations of antibodies raised against any antigens or portions of antigens can be used in accordance with the present invention.

The urine based VL capture ELISA diagnostic test, an embodiment of the present invention, has numerous advantages, some of which are: it is a non-invasive VL diagnostic test that can differentiate active disease from parasitic and from cured VL patients; should be a highly sensitive and specific test for VL; can aid a rapid diagnostic that will allow prompt therapy against VL, which is fatal if not treated promptly; should be an important tool to follow up therapy efficiently; should be useful for the diagnosis of VL in VL/HIV-co-infected patients, as the conventional serological diagnosis of VL in these patients is problematic and not sensitive.

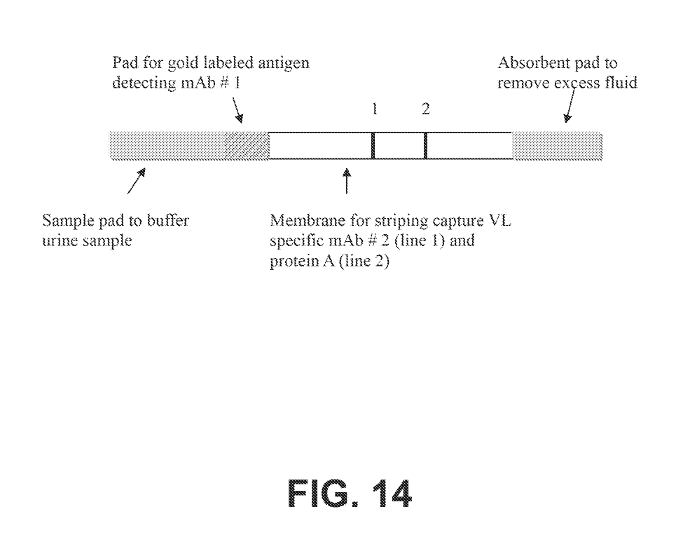

The solid support, such as a microtiter plate, dipstick, bead, pad, strip, or other suitable support, can be coated directly or indirectly with an anti-VL polypeptide antibody or VL specific antigen. For example, an anti-VL polypeptide antibody can coat a microtiter well, or a biotinylated anti-VL polypeptide Mab can be added to a streptavidin coated support. With respect to a immunochromatographic assay, a pad or strip can be coated with an antibody specific for the antigen, and when a sample having the one or more of antigens described herein comes into contact with the antibody, the complex can turn a color with aid of a detector, as further described herein. See FIG. 14. A variety of immobilizing or coating methods as well as a number of solid supports can be used, and can be selected according to the desired format.

In one immunochromatographic assay, a sample having the VL antigens of the present invention can be added to the pad like that shown in FIG. 14. The sample diffuses across a gold labeled antibody (mAb#1) that is specific to a portion of the antigen, and a complex between the antigen in the sample and the antibody is formed. As the complex further diffuses across the pad having a second antibody (mAb #2), the antibody binds to a different portion of the antigen. When it hits the line labeled “line 1”, the labeled complex turns a color, and the control line (“line 2”) will also change color. If the sample does not contain the VL antigens of the present invention, then line 1 will not turn color because the gold-labeled antibody will not bind to the sample and therefore will not come into contact with line 1.

In another embodiment, the sample (or an antigenic VL polypeptide standard) is combined with the solid support simultaneously with the detector antibody, and optionally with a one or more reagents by which detection is monitored. For example, the sample can be combined with the solid support simultaneously with (a) HRP-conjugated anti-VL polypeptide Mab, or (b) a biotinylated anti-VL polypeptide Mab and HRP-streptavidin.

A known amount of an antigenic VL polypeptide standard can be prepared and processed as described above for a suitable sample. This antigenic VL polypeptide standard assists in quantifying the amount of antigenic VL polypeptides detected by comparing the level of antigenic VL polypeptides in the sample relative to that in the standard.

A physician, technician, apparatus, or a qualified person can compare the amount of detected complex with a suitable control to determine if the levels are elevated. For example, the level of antigenic VL polypeptides following treatment can be compared with a baseline level prior to treatment, or with levels in normal individuals or suitable controls. A decrease in or maintenance of the levels of one or more VL polypeptides in the urine, as compared to baseline levels, indicates that the treatment is working, whereas increases in levels indicates that is not effective.

Typical assays for antigenic VL polypeptides are sequential assays in which a plate is coated with first antibody, sample is added, the plate is washed, second tagged antibody is added, and the plate is washed and bound second antibody is quantified. In another embodiment, a format in which antibodies and the sample are added simultaneously, in a competitive ELISA format, can achieve greater sensitivity.

A variety of methods can determine the amount of antigenic VL polypeptides in complexes. For example, when HRP is used as a label, a suitable substrate such as OPD can be added to produce color intensity directly proportional to the bound anti-VL polypeptides Mab (assessed e.g., by optical density), and therefore to the antigenic VL polypeptides in the sample.

A technician, physician, qualified person, or apparatus can compare the results to a suitable control such as a standard, or baseline levels of antigenic VL polypeptides in a sample from the same donor. For example, the assay can be performed using a known amount of antigenic VL polypeptides standard in lieu of a sample, and a standard curved established. One can relatively compare known amounts of the antigenic VL polypeptides standard to the amount of complex formed or detected.

The nucleic acid that encodes the antigenic VL polypeptides can also be assayed by hybridization, e.g., by hybridizing one of the VL sequences provided herein (e.g., SEQ ID NO: 1, 3, 5, 7, 9, 11, 13, 15, 17, 19, 21, or combinations thereof) or an oligonucleotide derived from one of the sequences, to a DNA or RNA-containing tissue sample from a person. Such a hybridization sequence can have a detectable label, e.g., radioactive, fluorescent, etc., attached to allow the detection of hybridization product. Such methods include contacting the sample with one or more oligonucleotide probes (e.g., at least about 15 contiguous bases) specific for the nucleic acid molecule described herein under high stringency conditions, sufficiently to allow hybridization between the sample and the probe; and detecting the nucleic acid molecule that hybridizes to the oligonucleotide probe in the sample. The presence of hybridization of the probe indicates VL infection, and the absence of hybridization indicates an absence of VL infection. Methods for hybridization are well known, and such methods are provided in U.S. Pat. No. 5,837,490, by Jacobs et al., the entire teachings of which are herein incorporated by reference in their entirety. The design of the oligonucleotide probe should preferably follow these parameters: (a) it should be designed to an area of the sequence which has the fewest ambiguous bases (“N's”), if any, and (b) it should be designed to have a Tₘ of approx. 80° Celsius (assuming 2° Celsius for each A or T and 4° C. for each G or C).

The present invention encompasses detection of VL polypeptides of the present invention in a sample using with PCR methods using primers disclosed or derived from sequences described herein. For example, the sequences described herein can be detected by PCR using SEQ ID Nos: 1, 3, 5, 7, 9, 11, 13, 15, 17, 19, 21, or combinations thereof. PCR is the selective amplification of a target sequence by repeated rounds of nucleic acid replication utilizing sequence-specific primers and a thermostable polymerase. PCR allows recovery of entire sequences between two ends of known sequence. Specifically, contacting the sample with at least two oligonucleotide primers in a PCR, wherein at least one of the oligonucleotide primers (e.g., at least about 10 contiguous bases) is specific for one or more of the isolated nucleic acid molecules described herein. The two are contacted sufficiently to allow amplification of the primers. The amplified nucleic acid sequence in the sample is detected. The presence any one of the amplified nucleic acid sequences indicate VL infection, and the absence of any one of the amplified nucleic acid sequences indicate an absence of VL infection. Methods of PCR are described herein and are known in the art.

Hence, the present invention includes kits for the detection of SEQ ID Nos:2, 4, 6, 8, 10, 12, 14, 16, 18, 20, 22, or combinations thereof, or the quantification of these sequences, having either antibodies specific for them or a portion thereof, or a nucleic acid sequence that can hybridize to the nucleic acid of encoding these sequences.

Additional immunological or nucleic acid assessments can be performed using methods known in the art. Assays, known in the art or those later developed can be used to assess the antigenic VL polypeptides in a sample.

In addition to measuring the presence of antigenic VL polypeptides in a sample, assays exist to determine the efficacy of a VL vaccine (e.g., the extent to which the immune response is stimulated). These types of assays can be used together to fully assess a person's VL status. For example, an individual who has a VL-specific immunogenic response, but tests negative to the presence of one or more the antigenic VL polypeptides in a sample, is one who has a level of immunity to the disease. However, a person who has a VL-specific immunogenic response and tests positive to the presence of the antigenic VL polypeptides of the present invention is someone who likely has VL.

The efficacy of a VL vaccine can be measured by determining the immunogenic response of the person who received the vaccine. The VL antigens of the present invention (and immunogenic portions thereof) described herein have the ability to induce an immunogenic response. More specifically, the antigens have the ability to induce proliferation and/or cytokine production (i.e., interferon-γ production) in T cells, B cells, and/or macrophages. See Example 2.

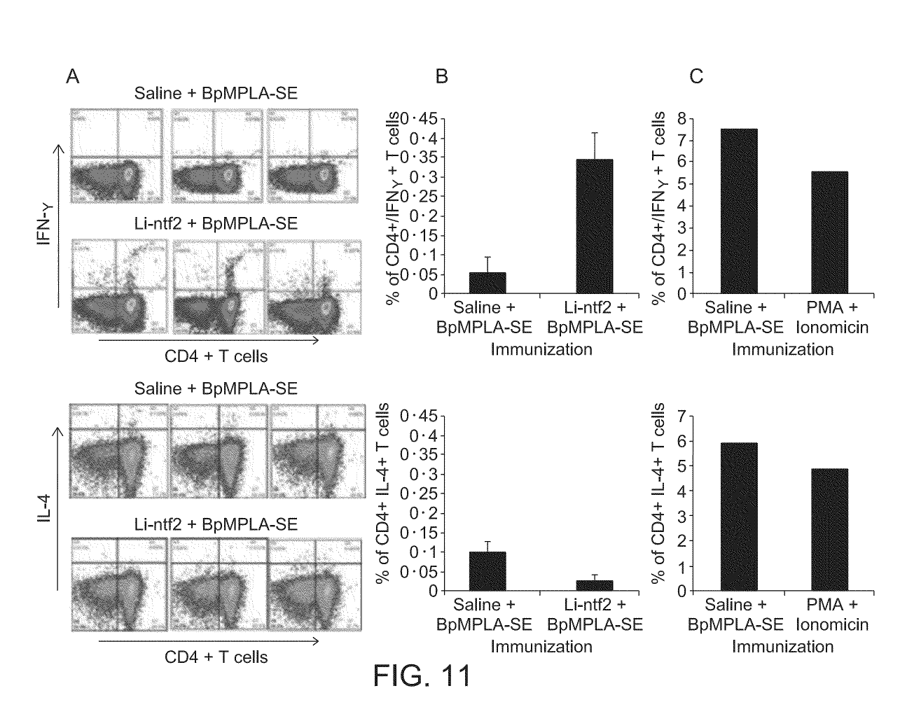

The selection of cell type for use in evaluating an immunogenic response to an antigen will, of course, depend on the desired response. Because CD4+ T cells of the Th1 phenotype are crucial mediators of resistance to VL, mice were immunized with a formulation containing the purified recombinant Li-ntf2, SEQ ID NO: 22, plus the adjuvant monophosphoryl lipid A (MPLA), as described in Example 2. After immunization, spleens were removed and stimulated with purified recombinant Li-ntf2. Cytokine responses (IFN-γ and IL-4) were measured along with the IgG1 and IgG2a specific antibody response to the recombinant Li-ntf2. High titers of both IgG1 and IgG2a were generated. Because class switch to IgG2a is dependent on production of IFN-γ, this result is consistent with a stimulation of Th1 response by the antigen/adjuvant formulation. In the cell cultures obtained from antigen immunized mice and stimulated in vitro with Li-ntf2 the Th1 cytokine IFN-γ was readily detected (FIG. 11, upper panels). Therefore, together these results confirm that the immunization protocol using the adjuvant BpMPLA-SE induces a preferential Th1 response to the L. infantum chagasi vaccine candidate Li-ntf2. An individual having asymptomatic VL will not generally not have symptoms of VL (e.g., chronic and irregular fever, abdominal pain, progressive emaciation, malaise, anemia, weight loss, hepatosplenomegaly (>2 cm from the costal margin)) but have results from laboratory tests that indicate the presences of VL. Alternatively or in addition, challenge experiments can be performed to determine the suitability of a vaccine. To determine protection, parasite enumeration can be performed, as was done in Example 2. As can be seen the immunizations performed in Example 2 with recombinant Li-ntf2 plus BpMPLA-SE, significant reduction in the parasite burden (1.3 log₁₀) occurred, as compared to animals immunized with saline+BpMPLA-SE.

T cells, B cells and macrophages derived from L. infantum-immune individuals can be prepared using methods known to those of ordinary skill in the art. For example, a preparation of PBMCs (i.e., peripheral blood mononuclear cells) can be employed without further separation of component cells. PBMCs can generally be prepared, for example, using density centrifugation through Ficoll (Winthrop Laboratories, N.Y.). T cells for use in the assays described herein can also be purified directly from PBMCs. Alternatively, an enriched T cell line reactive against VL proteins, or T cell clones reactive to individual VL proteins, can be employed. Such T cell clones can be generated by, for example, culturing PBMCs from L. infantum-immune individuals with VL proteins for a period of 2-4 weeks. This allows expansion of only the VL protein-specific T cells, resulting in a line composed solely of such cells. These cells can then be cloned and tested with individual proteins, using methods known to those of ordinary skill in the art, to more accurately define individual T cell specificity. In general, antigens that test positive in assays for proliferation and/or cytokine production (i.e., interferon-γ production) performed using T cells, B cells and/or macrophages derived from an L. infantum-immune individual are considered immunogenic. Such assays can be performed, for example, using the representative procedures described below. Immunogenic portions of such antigens can be identified using similar assays, and can be present within the polypeptides described herein.

The ability of a polypeptide (e.g., an immunogenic antigen, or a portion or other variant thereof) to induce cell proliferation can be evaluated by contacting the cells (e.g., T cells) with the polypeptide and measuring the proliferation of the cells. In general, the amount of polypeptide that is sufficient for evaluation of about 10⁵ cells ranges from about 10 ng/mL to about 100 μg/mL and preferably is about 10 μg/mL. The incubation of polypeptide with cells is typically performed at 37° C. for about six days. Following incubation with polypeptide, the cells are assayed for a proliferative response, which can be evaluated by methods known to those of ordinary skill in the art, such as exposing cells to a pulse of radiolabeled thymidine and measuring the incorporation of label into cellular DNA. In general, a polypeptide that results in at least a three-fold increase in proliferation above background (i.e., the proliferation observed for cells cultured without polypeptide) is considered to be able to induce proliferation.

The ability of a polypeptide to stimulate the production of interferon-γ in cells can be evaluated by contacting the cells with the polypeptide and measuring the level of interferon-γ produced by the cells, as demonstrated in Example 2. In general, the amount of polypeptide that is sufficient for the evaluation of about 10⁵ cells ranges from about 10 ng/mL to about 100 μg/mL and preferably is about 10 μg/mL. The polypeptide can, but need not, be immobilized on a solid support, such as a bead or a biodegradable microsphere, such as those described in U.S. Pat. Nos. 4,897,268 and 5,075,109. The incubation of polypeptide with the cells is typically performed at 37° C. for about six days. Following incubation with polypeptide, the cells are assayed for interferon-γ. (or one or more subunits thereof), which can be evaluated by methods known to those of ordinary skill in the art, such as an enzyme-linked immunosorbent assay (ELISA). In general, a polypeptide that results in the production of at least 50 pg of interferon-γ per mL of cultured supernatant (containing 10⁴-10⁵ T cells per mL) is considered able to stimulate the production of interferon-γ.

In general, immunogenic antigens are those antigens that stimulate proliferation and/or cytokine production (i.e., interferon-γ production) in T cells, B cells, and/or macrophages derived from at least about 25% of L. infantum-immune individuals. Among these immunogenic antigens, polypeptides having superior therapeutic properties can be distinguished based on the magnitude of the responses in the above assays and based on the percentage of individuals for which a response is observed. In addition, antigens having superior therapeutic properties will not stimulate proliferation and/or cytokine production in vitro in cells derived from more than about 25% of individuals who are not L. infantum-immune, thereby eliminating responses that are not specifically due to L. infantum-responsive cells. Those antigens that induce a response in a high percentage of T cell, B cell, and/or macrophage preparations from L. infantum-immune individuals (with a low incidence of responses in cell preparations from other individuals) have superior therapeutic properties.

### Fusion Proteins, Vaccine Compositions, Mode and Manner of Administration

The VL polypeptides of the present invention can be in the form of a conjugate or a fusion protein, which can be manufactured by known methods. In particular, 2 or more of the sequences, SEQ ID NO: 2, 4, 6, 8, 10, 12, 14, 16, 18, 20, or 22 can be fused to one another, or with other proteins, to provide a more effective vaccine composition, and stimulate an improved immunogenic response. Other proteins that can be used to make such a fusion protein include VL antigens that simulate the CD4+ T cell pathway of the immune response. Examples of such antigens include Antigen 85b, ESAT-6, MVL41, MVL39. Fusing a CD4+ T-cell pathway antigen with one of the polypeptides of the present invention can serve to increase effectiveness of the VL vaccine.

Fusion proteins can be manufactured according to known methods of recombinant DNA technology. For example, fusion proteins can be expressed from a nucleic acid molecule comprising sequences which code for a biologically active portion of the VL polypeptides or the entire VL polypeptides set forth in SEQ ID Nos:2, 4, 6, 8, 10, 12, 14, 16, 18, 20, 22, or combinations thereof, and its fusion partner, for example another sequence of the present invention, a portion of an immunoglobulin molecule, or another VL antigen from the CD4+ T cell pathway. For example, some embodiments can be produced by the intersection of a nucleic acid encoding immunoglobulin sequences into a suitable expression vector, phage vector, or other commercially available vectors. The resulting construct can be introduced into a suitable host cell for expression. Upon expression, the fusion proteins can be isolated or purified from a cell by means of an affinity matrix. By measurement of the alternations in the functions of transfected cells occurring as a result of expression of recombinant VL proteins, either the cells themselves or VL proteins produced from the cells can be utilized in a variety of screening assays.

As noted above, in certain aspects the inventive compositions comprise fusion proteins or DNA fusion molecules. Each fusion protein comprises a first and a second inventive polypeptide or, alternatively, a polypeptide of the present invention and a known VL antigen, together with variants of such fusion proteins. The fusion proteins of the present invention can also include a linker peptide between the first and second polypeptides. The DNA fusion molecules of the present invention comprise a first and a second isolated DNA molecule, each isolated DNA molecule encoding either an inventive VL antigen or a known VL antigen.

A DNA sequence encoding a fusion protein of the present invention is constructed using known recombinant DNA techniques to assemble separate DNA sequences encoding the first and second polypeptides into an appropriate expression vector, as described in detail below. The 3′ end of a DNA sequence encoding the first polypeptide is ligated, with or without a peptide linker, to the 5′ end of a DNA sequence encoding the second polypeptide so that the reading frames of the sequences are in phase to permit mRNA translation of the two DNA sequences into a single fusion protein that retains the biological activity of both the first and the second polypeptides.

A peptide linker sequence can be employed to separate the first and the second polypeptides by a distance sufficient to ensure that each polypeptide folds into its secondary and tertiary structures. Such a peptide linker sequence is incorporated into the fusion protein using standard techniques well known in the art. Suitable peptide linker sequences can be chosen based on the following factors: (1) their ability to adopt a flexible extended conformation; (2) their inability to adopt a secondary structure that could interact with functional epitopes on the first and second polypeptides; and (3) the lack of hydrophobic or charged residues that might react with the polypeptide functional epitopes. Preferred peptide linker sequences contain Gly, Asn and Ser residues. Other near neutral amino acids, such as Thr and Ala can also be used in the linker sequence. Amino acid sequences which can be usefully employed as linkers include those disclosed in Maratea et al., Gene 40:39-46, 1985; Murphy et al., Proc. Natl. Acad. Sci. USA 83:8258-8262, 1986; U.S. Pat. No. 4,935,233 and U.S. Pat. No. 4,751,180. The linker sequence can be from 1 to about 50 amino acids in length. Peptide sequences are not required when the first and second polypeptides have non-essential N-terminal amino acid regions that can be used to separate the functional domains and prevent steric interference.

The ligated DNA sequences are operably linked to suitable transcriptional or translational regulatory elements. The regulatory elements responsible for expression of DNA are located only 5′ to the DNA sequence encoding the first polypeptides. Similarly, stop codons require to end translation and transcription termination signals are only present 3′ to the DNA sequence encoding the second polypeptide.

Efficacy of a vaccine including the isolated sequences of the present invention can be determined based on the ability of the antigen to provide at least about a 50% (e.g., about a 60%, about a 70%, about a 80%, about a 90%, or about a 100%) reduction in parasite numbers and/or at least about a 40% (e.g., about a 50%, about a 60%, about a 70%, about a 80%, about a 90%, or about a 100%) decrease in mortality following experimental infection in a challenge experiment. Suitable experimental animals include mice, guinea pigs, and primates.

The compositions of the present invention are preferably formulated as either pharmaceutical compositions or as vaccines for in the induction of protective immunity against VL in a patient. A patient can be afflicted with a disease, or can be free of detectable disease and/or infection. In other words, protective immunity can be induced to prevent, reduce the severity of, or treat VL.

In one embodiment, pharmaceutical compositions of the present invention comprise one or more of the above polypeptides, either present as a mixture or in the form of a fusion protein, and a physiologically acceptable carrier. Similarly, vaccines comprise one or more the above polypeptides and a non-specific immune response enhancer, such as an adjuvant or a liposome (into which the polypeptide is incorporated).

In another embodiment, a pharmaceutical composition and/or vaccine of the present invention can contain one or more of the DNA molecules of the present invention, either present as a mixture or in the form of a DNA fusion molecule, each DNA molecule encoding a polypeptide as described above, such that the polypeptide is generated in situ. In such vaccines, the DNA can be present within any of a variety of delivery systems known to those of ordinary skill in the art, including nucleic acid expression systems, bacterial and viral expression systems. Appropriate nucleic acid expression systems contain the necessary DNA sequences for expression in the patient (such as a suitable promoter and terminating signal). Bacterial delivery systems involve the administration of a bacterium (such as Bacillus-Calmette-Guerrin) that expresses an immunogenic portion of the polypeptide on its cell surface. In a preferred embodiment, the DNA can be introduced using a viral expression system (e.g., vaccinia or other pox virus, retrovirus, or adenovirus), which can involve the use of a non-pathogenic (defective), replication competent virus. Techniques for incorporating DNA into such expression systems are well known to those of ordinary skill in the art. The DNA can also be “naked,” as described, for example, in Ulmer et al., Science 259:1745-1749, 1993 and reviewed by Cohen, Science 259:1691-1692, 1993. The uptake of naked DNA can be increased by coating the DNA onto biodegradable beads, which are efficiently transported into the cells.

The antigenic VL molecules of the present invention can be administered with or without a carrier. The terms “pharmaceutically acceptable carrier” or a “carrier” refer to any generally acceptable excipient or drug delivery composition that is relatively inert and non-toxic. Exemplary carriers include sterile water, salt solutions (such as Ringer's solution), alcohols, gelatin, talc, viscous paraffin, fatty acid esters, hydroxymethylcellulose, polyvinyl pyrolidone, calcium carbonate, carbohydrates (such as lactose, sucrose, dextrose, mannose, albumin, starch, cellulose, silica gel, polyethylene glycol (PEG), dried skim milk, rice flour, magnesium stearate, and the like. Suitable formulations and additional carriers are described in Remington's Pharmaceutical Sciences, (17^{th} Ed., Mack Pub. Co., Easton, Pa.). Such preparations can be sterilized and, if desired, mixed with auxiliary agents, e.g., lubricants, preservatives, stabilizers, wetting agents, emulsifiers, salts for influencing osmotic pressure, buffers, coloring, preservatives and/or aromatic substances and the like which do not deleteriously react with the active compounds. Typical preservatives can include, potassium sorbate, sodium metabisulfite, methyl paraben, propyl paraben, thimerosal, etc. The compositions can also be combined where desired with other active substances, e.g., enzyme inhibitors, to reduce metabolic degradation. A carrier (e.g., a pharmaceutically acceptable carrier) is preferred, but not necessary to administer the compound.

The composition can be a liquid solution, suspension, emulsion, tablet, pill, capsule, sustained release formulation, or powder. The method of administration can dictate how the composition will be formulated. For example, the composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc.

The antigenic VL molecules used in the invention can be administered intravenously, parenterally, intramuscular, subcutaneously, orally, nasally, topically, by inhalation, by implant, by injection, or by suppository. The composition can be administered in a single dose or in more than one dose over a period of time to confer the desired effect.

The actual effective amounts of compound or drug can vary according to the specific composition being utilized, the mode of administration and the age, weight and condition of the patient. For example, as used herein, an effective amount of the drug is an amount which reduces the number of parasites. Dosages for a particular individual patient can be determined by one of ordinary skill in the art using conventional considerations, (e.g. by means of an appropriate, conventional pharmacological protocol).

For enteral or mucosal application (including via oral and nasal mucosa), particularly suitable are tablets, liquids, drops, suppositories, or capsules. A syrup, elixir or the like can be used wherein a sweetened vehicle is employed. Liposomes, microspheres, and microcapsules are available and can be used.

Pulmonary administration can be accomplished, for example, using any of various delivery devices known in the art such as an inhaler. See. e.g., S. P. Newman (1984) in Aerosols and the Lung, Clarke and Davis (eds.), Butterworths, London, England, pp. 197-224; PCT Publication No. WO 92/16192; PCT Publication No. WO 91/08760.

For parenteral application, particularly suitable are injectable, sterile solutions, preferably oily or aqueous solutions, as well as suspensions, emulsions, or implants, including suppositories. In particular, carriers for parenteral administration include aqueous solutions of dextrose, saline, pure water, ethanol, glycerol, propylene glycol, peanut oil, sesame oil, polyoxyethylene-polyoxypropylene block polymers, and the like. Ampules are convenient unit dosages.

Biodegradable microspheres (e.g., polylactic galactide) can also be employed as carriers for the pharmaceutical compositions of this invention. Suitable biodegradable microspheres are disclosed, for example, in U.S. Pat. Nos. 4,897,268 and 5,075,109.

Any of a variety of adjuvants can be employed in the vaccines of this invention to enhance the immune response. Most adjuvants contain a substance designed to protect the antigen from rapid catabolism, such as aluminum hydroxide or mineral oil, and a nonspecific stimulator of immune responses. Suitable adjuvants are commercially available and include, for example, Freund's Incomplete Adjuvant and Freund's Complete Adjuvant (Difco Laboratories) and Merck Adjuvant 65 (Merck and Company, Inc., Rahway, N.J.). Other suitable adjuvants include alum, biodegradable microspheres, monophosphoryl lipid A and quil A.

In the inventive vaccines, it is preferred that the adjuvant induces an immune response comprising Th1 aspects. Suitable adjuvant systems include, for example, a combination of monophosphoryl lipid A, preferably monophosphoryl lipid A (MLP) together with an aluminum salt. An enhanced system involves the combination of a monophosphoryl lipid A and a saponin derivative, particularly the combination of 3D-MLP and the saponin QS21 as disclosed in WO 94/00153, or a less reactogenic composition where the QS21 is quenched with cholesterol as disclosed in WO 96/33739. Previous experiments have demonstrated a clear synergistic effect of combinations of 3D-MLP and QS21 in the induction of both humoral and Th1 type cellular immune responses. A particularly potent adjuvant formation involving QS21, 3D-MLP and tocopherol in an oil-in-water emulsion is described in WO 95/17210 and is a preferred formulation.

The administration of the antigenic VL polypeptide molecules of the present invention and other compounds can occur simultaneously or sequentially in time. A DNA vaccine and/or pharmaceutical composition as described above can be administered simultaneously with or sequentially to an additional polypeptide of the present invention, a known VL antigen, an immune enhancer, or other compound known in the art that would be administered with such a vaccine. The compound can be administered before, after or at the same time as the antigenic VL molecules. Thus, the term “co-administration” is used herein to mean that the antigenic VL molecules and the additional compound (e.g., immune stimulating compound) will be administered at times to achieve a specific VL immune response, as described herein. The methods of the present invention are not limited to the sequence in which the compounds are administered, so long as the compound is administered close enough in time to produce the desired effect.

Routes and frequency of administration of the inventive pharmaceutical compositions and vaccines, as well as dosage, will vary from individual to individual and can parallel those currently being used in immunization using BCG. In general, the pharmaceutical compositions and vaccines can be administered by injection (e.g., intracutaneous, intramuscular, intravenous, or subcutaneous), intranasally (e.g., by aspiration), intralung, or orally. Between 1 and 3 doses can be administered for a 1-36 week period. Preferably, 3 doses are administered, at intervals of 3-4 months, and booster vaccinations can be given periodically thereafter. Alternate protocols can be appropriate for individual patients. A suitable dose is an amount of polypeptide or DNA that, when administered as described above, is capable of raising an immune response in an immunized patient sufficient to protect the patient from VL infection for at least 1-2 years. In general, the amount of polypeptide present in a dose (or produced in situ by the DNA in a dose) ranges from about 1 pg to about 100 mg per kg of host, typically from about 10 pg to about 1 mg, and preferably from about 100 pg to about 1 μg. Suitable dose sizes will vary with the size of the patient, but will typically range from about 0.1 mL to about 5 mL.

### Polypeptides and Their Function

The present invention relates to isolated polypeptide molecules that have been isolated including antigenic portions of VL sequences isolated from urine of infected patients. The present invention includes polypeptide molecules that contain the sequence of any one of the antigenic VL amino acid sequences (SEQ ID NO: 2, 4, 6, 8, 10, 12, 14, 16, 18, 20, 22, or combinations thereof). See FIGS. 7 and 13. The present invention also pertains to polypeptide molecules that are encoded by nucleic acid sequences, SEQ ID NO: 1, 3, 5, 7, 9, 11, 13, 15, 17, 19, 21, or combinations thereof).

As used herein, the term “polypeptide” encompasses amino acid chains of any length, including full length proteins (i.e., antigens), wherein the amino acid residues are linked by covalent peptide bonds. Thus, a polypeptide comprising an immunogenic portion of a Leishmania infantum antigen can consist entirely of the immunogenic portion, or can contain additional sequences. The additional sequences can be derived from the native L. infantum antigen or can be heterologous, and such sequences can (but need not) be immunogenic. In general, the polypeptides disclosed herein are prepared in substantially pure form. Preferably, the polypeptides are at least about 80% pure, more preferably at least about 90% pure and most preferably at least about 99% pure.

Antigenic VL polypeptides of the present invention referred to herein as “isolated” are polypeptides that separated away from other proteins and cellular material of their source of origin. Isolated antigenic VL polypeptides, peptides derived by infection with the L. infantum parasite, include essentially pure protein, proteins produced by chemical synthesis, by combinations of biological and chemical synthesis and by recombinant methods. The proteins of the present invention have been isolated and characterized as to its physical characteristics using the procedures described in the Exemplification, and can be done using laboratory techniques for protein purification. Such techniques include, for example, salting out, immunoprecipation, column chromatography, high pressure liquid chromatography, and electrophoresis.

The compositions and methods of the present invention also encompass variants of the above polypeptides and DNA molecules. A polypeptide “variant,” as used herein, is a polypeptide that differs from the recited polypeptide only in conservative substitutions and/or modifications, such that the diagnostic, therapeutic, antigenic and/or immunogenic properties of the polypeptide are retained. A variant of a specific L. infantum antigen will therefore be useful in diagnosing active VL, or stimulate cell proliferation and/or IFN-γ in Th1 cells raised against that specific antigen. Polypeptide variants preferably exhibit at least about 70%, more preferably at least about 90% and most preferably at least about 95% homology to the identified polypeptides. For polypeptides with immunoreactive properties, variants can, alternatively, be identified by modifying the amino acid sequence of one of the above polypeptides, and evaluating the immunoreactivity of the modified polypeptide. Such modified sequences can be prepared and tested using, for example, the representative procedures described herein.

As used herein, a “conservative substitution” is one in which an amino acid is substituted for another amino acid that has similar properties, such that one skilled in the art of peptide chemistry would expect the secondary structure and hydropathic nature of the polypeptide to be substantially unchanged. In general, the following groups of amino acids represent conservative changes: (1) ala, pro, gly, glu, asp, gln, asn, ser, thr; (2) cys, ser, tyr, thr; (3) val, ile, leu, met, ala, phe; (4) lys, arg, his; and (5) phe, tyr, trp, his.

Variants can also, or alternatively, contain other modifications, including the deletion or addition of amino acids that have minimal influence on the diagnostic or antigenic properties, secondary structure and hydropathic nature of the polypeptide. For example, a polypeptide can be conjugated to a signal (or leader) sequence at the N-terminal end of the protein which co-translationally or post-translationally directs transfer of the protein. The polypeptide can also be conjugated to a linker or other sequence for ease of synthesis, purification or identification of the polypeptide (e.g., poly-His), or to enhance binding of the polypeptide to a solid support. For example, a polypeptide can be conjugated to an immunoglobulin Fc region.

The present invention also encompasses VL proteins and polypeptides, variants thereof, or those having amino acid sequences analogous to the amino acid sequences of antigenic VL polypeptides described herein. Such polypeptides are defined herein as antigenic VL analogs (e.g., homologues), or mutants or derivatives. “Analogous” or “homologous” amino acid sequences refer to amino acid sequences with sufficient identity of any one of the VL amino acid sequences so as to possess the biological activity (e.g., the ability to elicit a protective immune response to VL parasites) of any one of the native VL polypeptides. For example, an analog polypeptide can be produced with “silent” changes in the amino acid sequence wherein one, or more, amino acid residues differ from the amino acid residues of any one of the VL protein, yet still possesses the function or biological activity of the VL. Examples of such differences include additions, deletions or substitutions of residues of the amino acid sequence of VL. Also encompassed by the present invention are analogous polypeptides that exhibit greater, or lesser, biological activity of any one of the VL proteins of the present invention. Such polypeptides can be made by mutating (e.g., substituting, deleting or adding) one or more amino acid or nucleic acid residues to any of the isolated VL molecules described herein. Such mutations can be performed using methods described herein and those known in the art. In particular, the present invention relates to homologous polypeptide molecules having at least about 70% (e.g., 75%, 80%, 85%, 90% or 95%) identity or similarity with SEQ ID NO: 2, 4, 6, 8, 10, 12, 14, 16, 18, 20, 22, or combination thereof. Percent “identity” refers to the amount of identical nucleotides or amino acids between two nucleotides or amino acid sequences, respectfully. As used herein, “percent similarity” refers to the amount of similar or conservative amino acids between two amino acid sequences.

The polypeptides of the present invention include full length sequences, partial sequences, functional fragments and homologues, that allow for or assist in stimulating an immunogenic specific or protective immune response to VL. “Immunogenic,” as used herein, refers to the ability to elicit an immune response (e.g., cellular) in a patient, such as a human, and/or in a biological sample. In particular, antigens that are immunogenic (and immunogenic portions thereof) stimulate cell proliferation, and/or interferon-γ production in biological samples comprising one or more cells (e.g., T cells, B cells, and macrophages). Such cells are derived from an L. infantum-immune individual. Immunogenic portions of the antigens described herein can be prepared and identified using the techniques described herein. Other techniques, such as those summarized in Paul, Fundamental Immunology, 3d ed., Raven Press, 1993, pp. 243-247 and references cited therein, can be used. Such techniques include screening polypeptide portions of the native antigen for immunogenic properties. An immunogenic portion of a polypeptide is a portion that, within such assays, generates an immune response (e.g., proliferation, interferon-γ production) that is substantially similar to that generated by the full-length antigen. In other words, an immunogenic portion of an antigen can generate at least about 20%, and preferably about 100%, of the proliferation induced by the full length antigen in the model proliferation assay described herein. An immunogenic portion can also, or alternatively, stimulate the production of at least about 20%, and preferably about 100%, of the interferon-γ induced by the full length antigen in the model assay described herein. As used herein, “VL” or “VL disease” refers to the disease cause by the infection of L. infantum.

Homologous polypeptides can be determined using methods known to those of skill in the art. Initial homology searches can be performed at NCBI against the GenBank, EMBL and SwissProt databases using, for example, the BLAST network service. Altschuler, S. F., et al., J. Mol. Biol., 215:403 (1990), Altschuler, S. F., Nucleic Acids Res., 25:3389-3402 (1998). Computer analysis of nucleotide sequences can be performed using the MOTIFS and the FindPatterns subroutines of the Genetics Computing Group (GCG, version 8.0) software. Protein and/or nucleotide comparisons were performed according to Higgins and Sharp (Higgins, D. G. and Sharp, P. M., Gene, 73:237-244 (1988) e.g., using default parameters).

Additionally, the individual isolated polypeptides of the present invention are biologically active or functional and play various roles in the parasites as well. The present invention includes fragments of these isolated amino acid sequences, yet possess the function or biological activity of the sequence. For example, polypeptide fragments comprising deletion mutants of the antigenic VL proteins can be designed and expressed by well-known laboratory methods. Fragments, homologues, or analogous polypeptides can be evaluated for biological activity, as described herein.

The present invention also encompasses biologically active derivatives or analogs of the above described antigenic VL polypeptides, referred to herein as peptide mimetics. Mimetics can be designed and produced by techniques known to those of skill in the art. (see e.g., U.S. Pat. Nos. 4,612,132; 5,643,873 and 5,654,276). These mimetics can be based, for example, on a specific VL amino acid sequence and maintain the relative position in space of the corresponding amino acid sequence. These peptide mimetics possess biological activity similar to the biological activity of the corresponding peptide compound, but possess a “biological advantage” over the corresponding antigenic VL amino acid sequence with respect to one, or more, of the following properties: solubility, stability and susceptibility to hydrolysis and proteolysis.

Methods for preparing peptide mimetics include modifying the N-terminal amino group, the C-terminal carboxyl group, and/or changing one or more of the amino linkages in the peptide to a non-amino linkage. Two or more such modifications can be coupled in one peptide mimetic molecule. Modifications of peptides to produce peptide mimetics are described in U.S. Pat. Nos. 5,643,873 and 5,654,276. Other forms of the antigenic VL polypeptides, encompassed by the present invention, include those which are “functionally equivalent.” This term, as used herein, refers to any nucleic acid sequence and its encoded amino acid, which mimics the biological activity of the VL polypeptides and/or functional domains thereof.

### VL Nucleic Acid Sequences, Plasmids, Vectors and Host Cells

The present invention, in one embodiment, includes an isolated nucleic acid molecule having a sequence of SEQ ID NO: 1, 3, 5, 7, 9, 11, 13, 15, 17, 19, 21 or combinations thereof. See FIGS. 7 and 13. As used herein, the terms “DNA molecule” or “nucleic acid molecule” include both sense and anti-sense strands, cDNA, genomic DNA, recombinant DNA, RNA, and wholly or partially synthesized nucleic acid molecules. A nucleotide “variant” is a sequence that differs from the recited nucleotide sequence in having one or more nucleotide deletions, substitutions or additions. Such modifications can be readily introduced using standard mutagenesis techniques, such as oligonucleotide-directed site-specific mutagenesis as taught, for example, by Adelman et al. (DNA 2:183, 1983). Nucleotide variants can be naturally occurring allelic variants, or non-naturally occurring variants. Variant nucleotide sequences preferably exhibit at least about 70%, more preferably at least about 80% and most preferably at least about 90% homology to the recited sequence. Such variant nucleotide sequences will generally hybridize to the recited nucleotide sequence under stringent conditions. In one embodiment, “stringent conditions” refers to prewashing in a solution of 6×SSC, 0.2% SDS; hybridizing at 65° Celsius, 6×SSC, 0.2% SDS overnight; followed by two washes of 30 minutes each in 1×SSC, 0.1% SDS at 65° C., and two washes of 30 minutes each in 0.2×SSC, 0.1% SDS at 65° C.

The present invention also encompasses isolated nucleic acid sequences that encode VL polypeptides, and in particular, those which encode a polypeptide molecule having an amino acid sequence of SEQ ID NO: 2, 4, 6, 8, 10, 12, 14, 16, 18, 20, 22, or combinations thereof. These VL nucleic acid sequences encode polypeptides that stimulate a protective immunogenic response to the L. infantum parasite and/or are involved the functions further described herein.

As used herein, an “isolated” gene or nucleotide sequence which is not flanked by nucleotide sequences which normally (e.g., in nature) flank the gene or nucleotide sequence (e.g., as in genomic sequences) and/or has been completely or partially purified from other transcribed sequences (e.g., as in a cDNA or RNA library). Thus, an isolated gene or nucleotide sequence can include a gene or nucleotide sequence which is synthesized chemically or by recombinant means. Nucleic acid constructs contained in a vector are included in the definition of “isolated” as used herein. Also, isolated nucleotide sequences include recombinant nucleic acid molecules and heterologous host cells, as well as partially or substantially or purified nucleic acid molecules in solution. In vivo and in vitro RNA transcripts of the present invention are also encompassed by “isolated” nucleotide sequences. Such isolated nucleotide sequences are useful for the manufacture of the encoded antigenic VL polypeptide, as probes for isolating homologues sequences (e.g., from other mammalian species or other organisms), for gene mapping (e.g., by in situ hybridization), or for detecting the presence (e.g., by Southern blot analysis) or expression (e.g., by Northern blot analysis) of related genes in cells or tissue.

The antigenic VL nucleic acid sequences of the present invention include homologues nucleic acid sequences. “Analogous” or “homologous” nucleic acid sequences refer to nucleic acid sequences with sufficient identity of any one of the VL nucleic acid sequences, such that once encoded into polypeptides, they possess the biological activity of any one of the antigenic VL polypeptides described herein. For example, an analogous nucleic acid molecule can be produced with “silent” changes in the sequence wherein one, or more, nucleotides differ from the nucleotides of any one of the VL polypeptides described herein, yet, once encoded into a polypeptide, still possesses its function or biological activity. Examples of such differences include additions, deletions or substitutions. Also encompassed by the present invention are nucleic acid sequences that encode analogous polypeptides that exhibit greater, or lesser, biological activity of the VL proteins of the present invention. In particular, the present invention is directed to nucleic acid molecules having at least about 70% (e.g., 75%, 80%, 85%, 90% or 95%) identity with SEQ ID NO: 1, 3, 5, 7, 9, 11, 13, 15, 17, 19, 21, or combinations thereof.

The nucleic acid molecules of the present invention, including the full length sequences, the partial sequences, functional fragments and homologues, once encoded into polypeptides, elicit a specific immunogenic VL response, or has the function of the polypeptide, as further described herein. The homologous nucleic acid sequences can be determined using methods known to those of skill in the art, and by methods described herein including those described for determining homologous polypeptide sequences. Immunogenic antigens can then be sequenced using techniques such as Edman chemistry. See Edman and Berg, Eur. J. Biochem. 80:116-132, 1967.

Also encompassed by the present invention are nucleic acid sequences, DNA or RNA, which are substantially complementary to the DNA sequences encoding the antigenic VL polypeptides of the present invention, and which specifically hybridize with their DNA sequences under conditions of stringency known to those of skill in the art. As defined herein, substantially complementary means that the nucleic acid need not reflect the exact sequence of the VL sequences, but must be sufficiently similar in sequence to permit hybridization with VL nucleic acid sequence under high stringency conditions. For example, non-complementary bases can be interspersed in a nucleotide sequence, or the sequences can be longer or shorter than the VL nucleic acid sequence, provided that the sequence has a sufficient number of bases complementary to the VL sequence to allow hybridization therewith. Conditions for stringency are described in e.g., Ausubel, F. M., et al., Current Protocols in Molecular Biology, (Current Protocol, 1994), and Brown, et al., Nature, 366:575 (1993); and further defined in conjunction with certain assays.

Also encompassed by the present invention are nucleic acid sequences, genomic DNA, cDNA, RNA or a combination thereof, which are substantially complementary to the DNA sequences of the present invention and which specifically hybridize with the antigenic VL nucleic acid sequences under conditions of sufficient stringency (e.g., high stringency) to identify DNA sequences with substantial nucleic acid identity.

The present invention also includes portions and other variants of L. infantum antigens that are generated by synthetic or recombinant means. Synthetic polypeptides having fewer than about 100 amino acids, and generally fewer than about 50 amino acids, can be generated using techniques well known to those of ordinary skill in the art. For example, such polypeptides can be synthesized using any of the commercially available solid-phase techniques, such as the Merrifield solid-phase synthesis method, where amino acids are sequentially added to a growing amino acid chain. See Merrifield, J. Am. Chem. Soc. 85:2149-2146, 1963. Equipment for automated synthesis of polypeptides is commercially available from suppliers such as Applied BioSystems, Inc., Foster City, Calif., and can be operated according to the manufacturer's instructions. Variants of a native antigen can generally be prepared using standard mutagenesis techniques, such as oligonucleotide-directed site-specific mutagenesis. Sections of the DNA sequence can also be removed using standard techniques to permit preparation of truncated polypeptides.

In another embodiment, the present invention includes nucleic acid molecules (e.g., probes or primers) that hybridize to the VL sequences, SEQ ID NO:1, 3, 5, 7, 9, 11, 13, 15, 17, 19, 21, or combinations thereof under high or moderate stringency conditions. In one aspect, the present invention includes molecules that are or hybridize to at least about 20 contiguous nucleotides or longer in length (e.g., 50, 100, 200, 300, 400, 500, 600, 700, 800, 900, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, or 4000). Such molecules hybridize to one of the VL nucleic acid sequences under high stringency conditions. The present invention includes such molecules and those that encode a polypeptide that has the functions or biological activity described herein.

Typically the nucleic acid probe comprises a nucleic acid sequence (e.g. SEQ ID NO: 1, 3, 5, 7, 9, 11, 13, 15, 17, 19, 21, or combinations thereof) and is of sufficient length and complementarity to specifically hybridize to a nucleic acid sequence that encodes a VL antigenic polypeptide. For example, a nucleic acid probe can be at least about 5%, 10%, 20%, 30%, 40%, 50%, 60%, 70%, 80% or 90% the length of the VL nucleic acid sequence. The requirements of sufficient length and complementarity can be easily determined by one of skill in the art. Suitable hybridization conditions (e.g., high stringency conditions) are also described herein. Additionally, the present invention encompasses fragments of the polypeptides of the present invention or nucleic acid sequences that encodes a polypeptide wherein the polypeptide has the biologically activity of the VL polypeptides recited herein.

Such fragments are useful as probes for assays described herein, and as experimental tools, or in the case of nucleic acid fragments, as primers. A preferred embodiment includes primers and probes which selectively hybridize to the nucleic acid constructs encoding any one of the recited VL polypeptides. For example, nucleic acid fragments which encode any one of the domains described herein are also implicated by the present invention.

Stringency conditions for hybridization refers to conditions of temperature and buffer composition which permit hybridization of a first nucleic acid sequence to a second nucleic acid sequence, wherein the conditions determine the degree of identity between those sequences which hybridize to each other. Therefore, “high stringency conditions” are those conditions wherein only nucleic acid sequences which are very similar to each other will hybridize. The sequences can be less similar to each other if they hybridize under moderate stringency conditions. Still less similarity is needed for two sequences to hybridize under low stringency conditions. By varying the hybridization conditions from a stringency level at which no hybridization occurs, to a level at which hybridization is first observed, conditions can be determined at which a given sequence will hybridize to those sequences that are most similar to it. The precise conditions determining the stringency of a particular hybridization include not only the ionic strength, temperature, and the concentration of destabilizing agents such as formamide, but also factors such as the length of the nucleic acid sequences, their base composition, the percent of mismatched base pairs between the two sequences, and the frequency of occurrence of subsets of the sequences (e.g., small stretches of repeats) within other non-identical sequences. Washing is the step in which conditions are set so as to determine a minimum level of similarity between the sequences hybridizing with each other. Generally, from the lowest temperature at which only homologous hybridization occurs, a 1% mismatch between two sequences results in a 1° C. decrease in the melting temperature (Tₘ) for any chosen SSC concentration. Generally, a doubling of the concentration of SSC results in an increase in the Tₘ of about 17° C. Using these guidelines, the washing temperature can be determined empirically, depending on the level of mismatch sought. Hybridization and wash conditions are explained in Current Protocols in Molecular Biology (Ausubel, F. M. et al., eds., John Wiley & Sons, Inc., 1995, with supplemental updates) on pages 2.10.1 to 2.10.16, and 6.3.1 to 6.3.6.

High stringency conditions can employ hybridization at either (1) 1×SSC (10×SSC=3 M NaCl, 0.3 M Na₃-citrate . . . 2H₂O (88 g/liter), pH to 7.0 with 1 M HCl), 1% SDS (sodium dodecyl sulfate), 0.1-2 mg/ml denatured calf thymus DNA at 65° C., (2) 1×SSC, 50% formamide, 1% SDS, 0.1-2 mg/ml denatured calf thymus DNA at 42° C., (3) 1% bovine serum albumin (fraction V), 1 mM Na₂ . . . EDTA, 0.5 M NaHPO₄ (pH 7.2) (1 M NaHPO₄=134 g Na₂HPO₄ . . . 7H₂O, 4 ml 85% H₃PO₄ per liter), 7% SDS, 0.1-2 mg/ml denatured calf thymus DNA at 65° C., (4) 50% formamide, 5×SSC, 0.02 M Tris-HCl (pH 7.6), 1×Denhardt's solution (100X=10 g Ficoll 400, 10 g polyvinylpyrrolidone, 10 g bovine serum albumin (fraction V), water to 500 ml), 10% dextran sulfate, 1% SDS, 0.1-2 mg/ml denatured calf thymus DNA at 42° C., (5) 5×SSC, 5×Denhardt's solution, 1% SDS, 100 μg/ml denatured calf thymus DNA at 65° C., or (6) 5×SSC, 5×Denhardt's solution, 50% formamide, 1% SDS, 100 μg/ml denatured calf thymus DNA at 42° C., with high stringency washes of either (1) 0.3-0.1×SSC, 0.1% SDS at 65° C., or (2) 1 mM Na₂EDTA, 40 mM NaHPO₄ (pH 7.2), 1% SDS at 65° C. The above conditions are intended to be used for DNA-DNA hybrids of 50 base pairs or longer. Where the hybrid is believed to be less than 18 base pairs in length, the hybridization and wash temperatures should be 5-10° C. below that of the calculated Tₘ of the hybrid, where Tₘ in ° C.=(2× the number of A and T bases)+(4× the number of G and C bases). For hybrids believed to be about 18 to about 49 base pairs in length, the Tₘ in ° C.=(81.5° C.+16.6(log₁₀M)+0.41(% G+C)−0.61 (% formamide)−500/L), where “M” is the molarity of monovalent cations (e.g., Na⁺), and “L” is the length of the hybrid in base pairs.

Moderate stringency conditions can employ hybridization at either (1) 4×SSC, (10×SSC=3 M NaCl, 0.3 M Na₃-citrate . . . 2H₂O (88 g/liter), pH to 7.0 with 1 M HCl), 1% SDS (sodium dodecyl sulfate), 0.1-2 mg/ml denatured calf thymus DNA at 65° C., (2) 4×SSC, 50% formamide, 1% SDS, 0.1-2 mg/ml denatured calf thymus DNA at 42° C., (3) 1% bovine serum albumin (fraction V), 1 mM Na₂ . . . EDTA, 0.5 M NaHPO₄ (pH 7.2) (1 M NaHPO₄=134 g Na₂HPO₄ . . . 7H₂O, 4 ml 85% H₃PO₄ per liter), 7% SDS, 0.1-2 mg/ml denatured calf thymus DNA at 65° C., (4) 50% formamide, 5×SSC, 0.02 M Tris-HCl (pH 7.6), 1×Denhardt's solution (100X=10 g Ficoll 400, 10 g polyvinylpyrrolidone, 10 g bovine serum albumin (fraction V), water to 500 ml), 10% dextran sulfate, 1% SDS, 0.1-2 mg/ml denatured calf thymus DNA at 42° C., (5) 5×SSC, 5×Denhardt's solution, 1% SDS, 100 μg/ml denatured calf thymus DNA at 65° C., or (6) 5×SSC, 5×Denhardt's solution, 50% formamide, 1% SDS, 100 μg/ml denatured calf thymus DNA at 42° C., with moderate stringency washes of 1×SSC, 0.1% SDS at 65° C. The above conditions are intended to be used for DNA-DNA hybrids of 50 base pairs or longer. Where the hybrid is believed to be less than 18 base pairs in length, the hybridization and wash temperatures should be 5-10° C. below that of the calculated Tₘ of the hybrid, where Tₘ in ° C.=(2× the number of A and T bases)+(4× the number of G and C bases). For hybrids believed to be about 18 to about 49 base pairs in length, the Tₘ in ° C.=(81.5° C.+16.6(log₁₀M)+0.41(% G+C)−0.61 (% formamide)−500/L), where “M” is the molarity of monovalent cations (e.g., Na⁺), and “L” is the length of the hybrid in base pairs.

Low stringency conditions can employ hybridization at either (1) 4×SSC, (10×SSC=3 M NaCl, 0.3 M Na₃-citrate . . . 2H₂O (88 g/liter), pH to 7.0 with 1 M HCl), 1% SDS (sodium dodecyl sulfate), 0.1-2 mg/ml denatured calf thymus DNA at 50° C., (2) 6×SSC, 50% formamide, 1% SDS, 0.1-2 mg/ml denatured calf thymus DNA at 40° C., (3) 1% bovine serum albumin (fraction V), 1 mM Na₂ . . . EDTA, 0.5 M NaHPO₄ (pH 7.2) (1 M NaHPO₄=134 g Na₂HPO₄ . . . 7H₂O, 4 ml 85% H₃PO₄ per liter), 7% SDS, 0.1-2 mg/ml denatured calf thymus DNA at 50° C., (4) 50% formamide, 5×SSC, 0.02 M Tris-HCl (pH 7.6), 1×Denhardt's solution (100X=10 g Ficoll 400, 10 g polyvinylpyrrolidone, 10 g bovine serum albumin (fraction V), water to 500 ml), 10% dextran sulfate, 1% SDS, 0.1-2 mg/ml denatured calf thymus DNA at 40° C., (5) 5×SSC, 5×Denhardt's solution, 1% SDS, 100 μg/ml denatured calf thymus DNA at 50° C., or (6) 5×SSC, 5×Denhardt's solution, 50% formamide, 1% SDS, 100 μg/ml denatured calf thymus DNA at 40° C., with low stringency washes of either 2×SSC, 0.1% SDS at 50° C., or (2) 0.5% bovine serum albumin (fraction V), 1 mM Na₂EDTA, 40 mM NaHPO₄ (pH 7.2), 5% SDS. The above conditions are intended to be used for DNA-DNA hybrids of 50 base pairs or longer. Where the hybrid is believed to be less than 18 base pairs in length, the hybridization and wash temperatures should be 5-10° C. below that of the calculated Tₘ of the hybrid, where Tₘ in ° C.=(2× the number of A and T bases)+(4× the number of G and C bases). For hybrids believed to be about 18 to about 49 base pairs in length, the Tₘ in ° C.=(81.5° C.+16.6(log₁₀M)+0.41(% G+C)−0.61 (% formamide)−500/L), where “M” is the molarity of monovalent cations (e.g., Na.+), and “L” is the length of the hybrid in base pairs.

The VL nucleic acid sequences of the present invention, or a fragment thereof, can also be used to isolate additional homologs. For example, a cDNA or genomic DNA library from the appropriate organism can be screened with labeled VL nucleic acid sequence to identify homologous genes as described in e.g., Ausebel, et al., Eds., Current Protocols In Molecular Biology, John Wiley & Sons, New York (1997).

Immunogenic antigens can be produced recombinantly using a DNA sequence that encodes the antigen, which has been inserted into an expression vector and expressed in an appropriate host cell. DNA sequences encoding L. infantum antigens can, for example, be identified by screening an appropriate L. infantum genomic or cDNA expression library with sera obtained from patients infected with L. infantum. Such screens can generally be performed using techniques well known to those of ordinary skill in the art, such as those described in Sambrook et al., Molecular Cloning: A Laboratory Manual, Cold Spring Harbor Laboratories, Cold Spring Harbor, N.Y., 2001. Degenerate oligonucleotide sequences for use in such a screen can be designed and synthesized, and the screen can be performed. Polymerase chain reaction (PCR) can also be employed, using the above oligonucleotides in methods well known in the art, to isolate a nucleic acid probe from a cDNA or genomic library. The library screen can then be performed using the isolated probe. The present method can optionally include a labeled VL antigenic probe.

Alternatively, genomic or cDNA libraries derived from L. infantum can be screened directly using peripheral blood mononuclear cells (PBMCs) or T cell lines or clones derived from one or more L. infantum-immune individuals. In general, PBMCs and/or T cells for use in such screens can be prepared as described below. Direct library screens can generally be performed by assaying pools of expressed recombinant proteins for the ability to induce proliferation and/or interferon-γ production in T cells derived from an L. infantum-immune individual.

The invention also provides vectors, plasmids or viruses containing one or more of the VL nucleic acid molecules (e.g., having the sequence of SEQ ID NO: 1, 3, 5, 7, 9, 11, 13, 15, 17, 19, 21, or combinations thereof). Suitable vectors for use in eukaryotic and prokaryotic cells are known in the art and are commercially available or readily prepared by a skilled artisan. Additional vectors can also be found, for example, in Ausubel, F. M., et al., Current Protocols in Molecular Biology, (John Wiley & Sons, 2004) and Sambrook et al., “Molecular Cloning: A Laboratory Manual,” (2001).

Recombinant polypeptides containing portions and/or variants of a native antigen can be readily prepared from a DNA sequence encoding the polypeptide using a variety of techniques well known to those of ordinary skill in the art. For example, supernatants from suitable host/vector systems which secrete recombinant protein into culture media can be first concentrated using a commercially available filter. Following concentration, the concentrate can be applied to a suitable purification matrix such as an affinity matrix or an ion exchange resin. Finally, one or more reverse phase HPLC steps can be employed to further purify a recombinant protein.

Any of a variety of expression vectors known to those of ordinary skill in the art can be employed to express recombinant polypeptides of this invention. Expression can be achieved in any appropriate host cell that has been transformed or transfected with an expression vector containing a DNA molecule that encodes a recombinant polypeptide. Suitable host cells include prokaryotes, yeast and higher eukaryotic cells. Preferably, the host cells employed are E. coli, yeast or a mammalian cell line such as COS or CHO. The DNA sequences expressed in this manner can encode naturally occurring antigens, portions of naturally occurring antigens, or other variants thereof.

Uses of plasmids, vectors or viruses containing the cloned VL receptors or receptor fragments include one or more of the following; (1) generation of hybridization probes for detection and measuring level of VL or isolation of VL homologs; (2) generation of VL mRNA or protein in vitro or in vivo; and (3) generation of transgenic non-human animals or recombinant host cells.

In one embodiment, the present invention encompasses host cells transformed with the plasmids, vectors or viruses described above. Nucleic acid molecules can be inserted into a construct which can, optionally, replicate and/or integrate into a recombinant host cell, by known methods. The host cell can be a eukaryote or prokaryote and includes, for example, yeast (such as Pichia pastorius or Saccharomyces cerevisiae), bacteria (such as E. coli, L. infantum, or Bacillus subtilis), animal cells or tissue, insect Sf9 cells (such as baculoviruses infected SF9 cells) or mammalian cells (somatic or embryonic cells, Human Embryonic Kidney (HEK) cells, Chinese hamster ovary cells, HeLa cells, human 293 cells and monkey COS-7 cells). Host cells suitable in the present invention also include a mammalian cell, a bacterial cell, a yeast cell, an insect cell, and a plant cell.

The nucleic acid molecule can be incorporated or inserted into the host cell by known methods. Examples of suitable methods of transfecting or transforming cells include calcium phosphate precipitation, electroporation, microinjection, infection, lipofection and direct uptake. “Transformation” or “transfection” as used herein refers to the acquisition of new or altered genetic features by incorporation of additional nucleic acids, e.g., DNA. “Expression” of the genetic information of a host cell is a term of art which refers to the directed transcription of DNA to generate RNA which is translated into a polypeptide. Methods for preparing such recombinant host cells and incorporating nucleic acids are described in more detail in Ausubel, F. M., et al., Current Protocols in Molecular Biology, (John Wiley & Sons, 2004) and Sambrook et al., “Molecular Cloning: A Laboratory Manual,” (2001), for example.

The host cell is then maintained under suitable conditions for expression and recovery of the antigenic VL polypeptide of the present invention. Generally, the cells are maintained in a suitable buffer and/or growth medium or nutrient source for growth of the cells and expression of the gene product(s). The growth media are not critical to the invention, are generally known in the art and include sources of carbon, nitrogen and sulfur. Examples include Luria broth, Superbroth, Dulbecco's Modified Eagles Media (DMEM), RPMI-1640, M199 and Grace's insect media. The growth media can contain a buffer, the selection of which is not critical to the invention. The pH of the buffered Media can be selected and is generally one tolerated by or optimal for growth for the host cell.

The host cell is maintained under a suitable temperature and atmosphere. Alternatively, the host cell is aerobic and the host cell is maintained under atmospheric conditions or other suitable conditions for growth. The temperature should also be selected so that the host cell tolerates the process and can be for example, between about 13-40 degree Celsius.

### EXEMPLIFICATION

### Example 1

### L. Infantum Proteins in Urine of VL Patients

Despite the clear need to control visceral leishmaniasis (VL), the existing diagnostic tests have serious shortcomings. Here, we introduce an innovative approach to directly identify Leishmania infantum antigens produced in vivo in humans with VL. We combined reverse-phase high-performance liquid chromatography (RP-HPLC) with mass spectrometry and categorized three distinct L. infantum proteins presumably produced in bone marrow/spleen/liver and excreted in the urine of patients with VL. The genes coding for these proteins (L. infantum iron superoxide dismutase, NCBI accession number XP_{—}001467866.1; L. infantum tryparedoxin, NCBI accession number XP_{—}001466642.1; and L. infantum nuclear transport factor 2, NCBI accession number XP_{—}001463738.1) were cloned and the recombinant molecules were produced in Escherichia coli. Antibodies to these proteins were produced in rabbits and chickens and were used to develop a capture enzyme-linked immunosorbant assay (ELISA) designed to detect these L. infantum antigens in the urine of VL patients. Specificity of the antibodies was confirmed by Western blot analysis using both recombinant proteins and whole parasite extract. Importantly, a urinary antigen detection assay assembled with pairs of antibodies specific for each of these antigens identified 17 of 19 patients with VL. These results indicate that an improved antigen detection assay based on L. infantum proteins present in the urine of patients with VL represents an important new strategy for the development of a specific and accurate diagnostic test that has the potential to both distinguish active VL from asymptomatic infection as well as to serve as an important tool to monitor therapy efficacy.

### Materials and Methods

### Human Samples:

A total of 25 urine samples from patients with VL were evaluated in this study. These samples were collected from patients diagnosed with VL based on the following criteria: a clinical course consistent with VL (e.g., fever, anemia, hepatosplenomegaly), and confirmatory laboratory findings (identification of Leishmania in bone marrow aspirates). A single urine sample was collected from each patient at the time of his/her first visit to the hospital for diagnosis and treatment of VL. Urine specimens were stored frozen at −20° C. and subsequently shipped in dry ice to the Forsyth Institute, Cambridge, Mass. The patients were from the Natan Portella Institute of Tropical Diseases, Federal University of Piaui, Teresina, PI, Brazil. In addition, urine samples were obtained from healthy subjects (n=16), from patients with cutaneous leishmaniasis (n=10), Chagas' disease (n=8), schistosomiasis (n=14), and tuberculosis (n=10). These samples were from patients from the area of Montes Claros city, MG, Brazil, and were collected and confirmed by the Department of Parasitology, Federal University of Minas Gerais, Belo Horizonte, MG, Brazil. Urine donation protocols were approved by the Investigational Review Board and Ethics Committee of the Federal University of Piaui Medical School and Federal University of Minas Gerais respectively.

### Mass Spectroscopy Analysis:

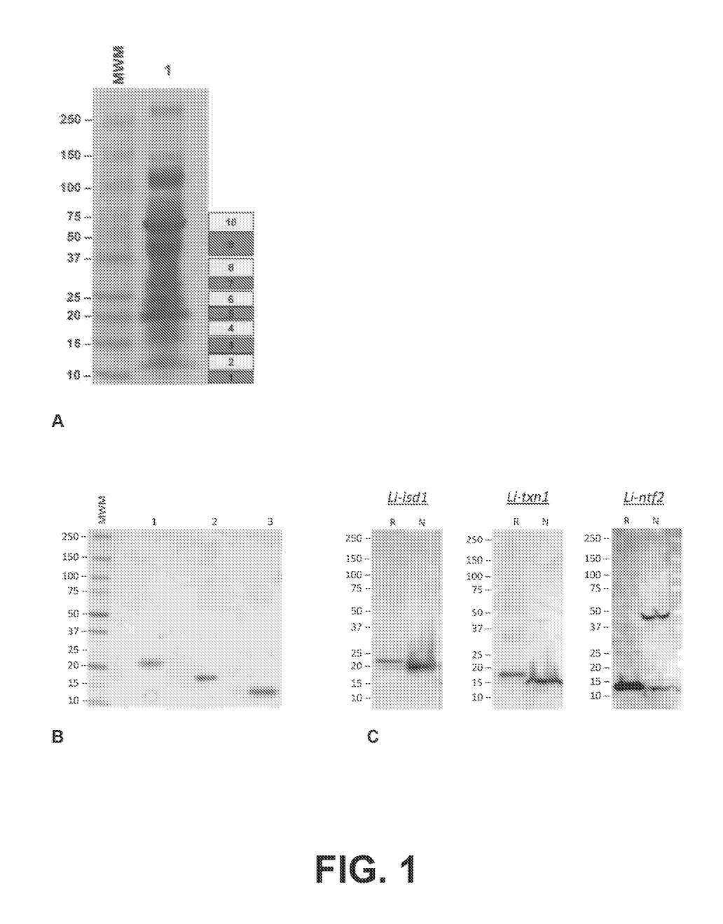

Frozen individual samples (15 ml) from five patients with confirmed VL were thawed, centrifuged, and filtered over 0.2-μm filters. Urine specimens were then concentrated using Centricon P3 (3-kDa cutoff filters) to ˜200-300 μl. Equal volumes of concentrated urine specimens were mixed with electrophoresis sample buffer and then submitted to SDS-PAGE followed by Coomassie staining Bands ranging from ˜10 kDa o ˜75 kDa were, in general excised from the gel and submitted for mass spectroscopy (MS) analysis at the Taplin Mass Spectrometry Facility, Harvard Medical School, Boston, Mass. FIG. 1, panel A illustrates a typical pattern of bands obtained for the concentrated urine of a patient with VL. This pattern was highly analogous to those of all other VL urine samples as well as to that obtained for urine specimens of two healthy control subjects. FIG. 1, panel A also illustrates the positions of the bands that were excised from the gel for further analyses. For each urine sample, 8 to 10 bands wre cut from the gel. Each band was then independently submitted to MS runs. Gel bands were trypsin-digested into peptides. Peptides were analyzed by nanoscale liquid chromatography coupled to a tandem mass spectrometer. Eluted peptides first had their molecular masses measured and then were fragmented, and finally the fragment masses were measured. The specific fragmentation pattern was computer-searched against predicted tryptic peptides from all known proteins from genome sequencing projects of human and Leishmania protozoa. The power of the technique is in its redundancy. Because many peptides are generated from the initial gel band, multiple matches to the protein of interest are detected. In this way, the protein identity is completely unambiguous. Peptide score cutoff values were chosen at cross-correlation (Xcorr) values of 1.8 for singly charged ions, 2.5 for doubly charged ions, and 3.0 for triply charged ions, along with the magnitutde of predicted fragment ion values (deltaCN) of 0.1 and final correlation score rank (RSP) values of 1. The cross correlation values chosen for each peptide ensure a high confidence match for the different charge states, while the deltaCN cutoff ensures the uniqueness of the top hit in the preliminary scoring and that the peptide fragment file matched only one protein hit.

### Cloning of the Leishmania Infantum Gene, Protein Expression, and Purification:

Oligonucleotide PCR primers were designed to amplify the full-length open reading frame of the target genes from genomic DNA of L. infantum. The forward primer contained an NdeI restriction site at the ATG initiation codon followed by sequences derived from the gene. The reverse primer included a BamHI restriction site followed by a stop codon and sequences from the gene. The resultant PCR products were digested with restriction enzymes and subcloned into the pET-14b expression vector, which was similarly digested for directional cloning. Alternatively, the DNA sequence was codon optimized for expression in E. coli containing the same restriction enzyme sites (NdeI and BamHI). The DNA fragment was synthesized (Blue Heron, Bothell, Wash.) and the synthetic gene was subcloned into pET-14b as well. A ligated pET-14b vector was subsequently used to transform E. coli BL21(DE3)pLysS host cells (Novagen, Madison, Wis.) for expression. Recombinant proteins were obtained and purified from 100 ml of isopropyl-β-D-thiogalactopyranoside (IPTG)-induced batch cultures by affinity chromatography using QIAexpress Ni-NTA agarose matrix (QIAGEN, Chatsworth, Calif.) as described previously (Kashino S. S., et al., Identification and characterization of Mycobacterium tuberculosis antigens in urine of patients with active pulmonary tuberculosis: an innovative and alternative approach of antigen discovery of useful microbial molecules, Clin. Exp. Immunol., 153: 56-62 (2008), Mukherjee S., et al., Cloning of the gene encoding a protective Mycobacterium tuberculosis secreted protein detected in vivo during the initial phases of the infectious process, J. Immunol., 175: 5298-5305 (2005)). The yields of recombinant proteins were 10 to 20 mg per liter of induced bacterial culture, and purity was assessed by SDS-PAGE followed by Coomassie blue staining Endotoxin levels present in the purified recombinant protein preparations were measured by the Limulus amebocyte lysate assay (Lonza, Walkersville, Md.) and shown to be <5 endotoxin units (EU)/mL.

### Generation of Specific Polyclonal Antibodies:

The purified recombinant protein (250 μg) was emulsified with equal volume of incomplete Freund adjuvant and injected at multiple subcutaneous (s.c.) sites into female New Zealand rabbits or New Hampshire Red chickens (Capralogics Inc., Hardwick, Mass.). The animals were given two s.c. boosters (250 μg of Ag in IFA) 3 weeks apart. One week after the final boost the animals were bled and sera (from the rabbits) and yolks (from the chicken eggs) were collected. IgG (from rabbits) and IgY (from chickens) were purified by standard affinity chromatography or by antigen immobilized on Sepharose 4B resin (cyanogen bromide [CNBr]-activated Sepharose 4B, GE Healthcare). A portion of the rabbit IgG was biotinylated with the EZ-Link-Sulfo-NHS-LC biotinylation kit from Thermo Fischer Scientific (Pittsburgh, Pa.) according to manufacturer's instructions. Between 1 and 2.5 molecules of biotin per IgG were invariably obtained for all the different batches.

### Western Blot:

Purified recombinant proteins (100 ng) and whole antigen extract from Leishmania infantum were fractionated by SDS-PAGE (4-20% gradient gel) and transferred to a polyvinylidene fluoride (PVDF) membrane (Millipore, Medford, Mass.). Crude Leishmania lysates were prepared from promastigote parasites cultured for 7 to 10 days in complete Schneider's medium at 26° C. (promastigote lysate preparation). The blots were blocked overnight at 4° C. with Tris-buffered saline with 0.1% Tween 20 (TBS-T) containing 1% bovine serum albumin (BSA) and subsequently probed with antigen specific rabbit antisera or pre-immune rabbit sera. After several rinses with TBS-T, goat anti-rabbit IgG labeled with horseradish peroxidase (Thermo Scientific Pierce, Rockford, Ill.) was added. After additional washings, bound conjugates were detected using the ECL enhanced chemiluminescence system (Amersham/GE Healthcare, Piscataway, N.J.) and proteins were visualized by autoradiography (Kodak BioMax, Rochester, N.Y.).

### Capture ELISA:

A capture ELISA antigen detection test was developed using purified IgG anti-L. infantum recombinant antigens obtained from antisera produced in two different rabbits or IgY produced in chickens Briefly, wells of 96 well ELISA plates (high-binding EIA/RIA plates; Corning International, Corning, N.Y.) were coated overnight at 4° C. with 0.2 μg of purified IgG (or IgY) diluted in bicarbonate buffer pH 9.0. Wells were washed with PBS+0.1% Tween-20 (Sigma Chemical Co., St. Luis, Mo.) and blocked at room temperature with PBS+1% BSA+0.1% Tween 20 (PBS/BSA/Tween) for 2 h. After washing, human urine samples were added and incubated overnight at 4° C. Plates were washed followed by incubation for 1 h with biotin labeled IgG (obtained from the second immunized rabbit) or biotin labeled IgY at 1/2,000, a dilution previously determined by conventional sandwich ELISA. Following several rinses in PBS/BSA/Tween, peroxidase-labeled streptavidin at a 1/2,000 dilution (BD Bioscience, Franklin Lakes, N.J.) was added for 30 minutes. The plates were then washed, and reactions were developed with 3,3′,5,5′-tetramethylbenzidine (TMB) substrate and read at 450 nm.

### Statistical Analysis:

Statistical significance was determined by unpaired t test for comparisons between two groups. Comparisons were performed using the Mann-Whitney Rank Sum Test (p values <0.05 were considered statistically significant).

### Results

Isolation of Three Unique L. Infantum Protein Antigens from the Urine of Patients with VL:

Urine was collected from five patients with active, parasitologically confirmed VL registered at the Natan Portella Institute of Tropical Medicine, Teresina, PI, Brazil. None of the enrolled patients had any clinical signs or symptoms or laboratory findings compatible with renal or urinary tract abnormalities. These exclusion criteria were important to rule out renal pathology, which can theoretically be a factor that would be biasing the finding of L. infantum antigens present in the patients' urine. None of the patients were under anti-leishmania therapy at the time of urine collection. Individual urine samples were analyzed by mass spectrometry generating a total of approximately 400 peptide sequences. Most sequences of the identified peptides had sequence homologies to those of human proteins. However, eight peptide sequences that had no known homologies with human proteins had sequence homologies to the deduced sequences of three different L. infantum proteins (Table 1). Two of these peptides (LNAAAESNSGLASK (SEQ ID NO: 2) and GGGEPSGPLASAIVDSFGSFASFK (SEQ ID NO: 4)) had sequences with perfect matching to the sequence of L. infantum iron superoxide dismutase (NCBI accession number XP_{—}001467866.1) at positions 40-53 and 87-110, respectively. Four other peptides (QNDMVDMSSLSGK (SEQ ID NO: 6), MPWLSIPFEK (SEQ ID NO: 8), QYKVESIPTLIGLNADTGDTVTTR (SEQ ID NO: 10), and VESIPTLIGLNADTGDTVTTR (SEQ ID NO: 12)) had sequences with perfect matching sequences to the sequence of L. infantum tryparedoxin (NCBI accession number XP_{—}001466642.1) at positions 17-29, 84-93, 103-126, and 106-126 respectively. Finally, two peptides (FANLGFTEAAFK (SEQ ID NO: 14) and EQVQGVDAIMAR (SEQ ID NO: 16)) had sequences with perfect and strong matching to the sequence of a putative L. infantum protein (nuclear transport factor 2, NCBI accession number XP_{—}001463738.1) at positions 40-51 and 52-63, respectively. Importantly, most of these L. infantum peptides were identified in two out of five urine samples, thus strongly validating these findings (Table 2). Finally, no leishmanial

TABLE 1
L. infantum peptides identified by mass spectroscopy in individual urines
of patients with visceral leishmaniasis

| | | Putative | | | Molecular | |
|---|---|---|---|---|---|---|
| | | L. infantum | | Gene | mass of | |
| | XCorr | peptide | NCBI | Length | Protein | |
| Peptide | value | donor protein | Annotation | (bp) | (kDa) | pl |
| LNAAAESNSGLASK | 3.602 | Iron superoxide | XM_001467829 | 588 | 21.53 | 8.78 |
| (SEQ ID NO: 2) | | dismutase | | | | |
| GGGEPSGPLASAIVDSFGSFASF | 5.274 | (Li-isd1) | | | | |
| K (SEQ ID NO: 4) | | | | | | |
| QNDMVDMSSLSGK | 2.813 | Tryparedoxin | XM_001466605 | 438 | 16.7 | 5.2 |
| (SEQ ID NO: 6) | | (Li-txn1) | | | | |
| MPWLSIPFEK (SEQ ID NO: 8) | 2.789 | | | | | |
| QYKVESIPTLIGLNADTGDTVTT | 3.630 | | | | | |
| R (SEQ ID NO: 10) | | | | | | |
| VESIPTLIGLNADTGDTVTTR | 4.168 | | | | | |
| (SEQ ID NO: 12) | | | | | | |
| FANLGFTEAAFK | 4.0849 | Nuclear | XM_001463701 | 375 | 13.89 | 4.9 |
| (SEQ ID NO: 14) | | transport | | | | |
| EQVQGVDAIMAR | | factor 2 | | | | |
| (SEQ ID NO: 16) | 3.1871 | (Li-ntf2) | | | | |

matching peptides were found in urine specimens of two control subjects processed in a manner identical to that of the urine specimens of the VL patients (not shown).

### Gene Cloning and Protein Expression/Purification and Characterization of Discovered L. Infantum Antigens:

The open reading frame of each of the full-length genes coding for each protein was amplified by PCR from L. infantum genomic DNA followed by sub-cloning into the pET-14b expression vector. The recombinant proteins L. infantum iron superoxide dismutase (Li-isd1) and L. infantum tryparedoxin (Li-txn1) were readily obtained. However, this cloning strategy resulted in no expression of the L. infantum nuclear transport (Linft2) protein. Nevertheless, using a synthetic gene with an optimized sequence for E. coli followed by subcloning in pET-14b resulted in excellent overexpression of the recombinant protein. Recombinant proteins were next purified using Ni-nitrilotriacetic acid (Ni-NTA) agarose resin and purity was assessed by SDS-PAGE with Coomassie blue staining and is illustrated in FIG. 1B. To validate the recombinant proteins as replicas of the native parasite molecules, Western blot analysis

TABLE 2
Occurrence of identified L. infantum peptides among the VL patients who donated urine for
MS analyses

| | | Presence of each peptide in each patient | | | | |
|---|---|---|---|---|---|---|
| | | (gender, age [yr]) | | | | |
| | | A | B | C | D | E |
| Protein | Peptide | (Female, 2) | (Male, 7) | (Male, 29) | (Female, 2) | (Female, 6) |
| Li-isd1 | LNAAAESNSGLASK (SEQ ID NO: 2) | + | | | | |
| | GGGEPSGPLASAIVDSFGSFASFK | + | | | | |
| | (SEQ ID NO: 4) | | | | | |
| Li-txn1 | QNDMVDMSSLSGK (SEQ ID NO: 6) | | + | | + | |
| | MPWLSIPFEK (SEQ ID NO: 8) | | + | | + | |
| | QYKVESIPTLIGLNADTGDTVTTR | | + | | + | |
| | (SEQ ID NO: 10) | | | | | |
| | VESIPTLIGLNADTGDTVTTR | | + | | + | |
| | (SEQ ID NO: 12) | | | | | |
| Li-ntf2 | FANLGFTEAAFK (SEQ ID NO: 14) | | | + | | + |
| | EQVQGVDAIMAR (SEQ ID NO: 16) | | | | | + |

was carried out using specific rabbit IgG antibodies. A crude lysate of the parasite and the purified recombinant proteins were immunobloted and then probed with the specific polyclonal rabbit antisera (FIG. 1C). All three antisera recognized a single band in the crude parasite lysate that migrated at a molecular mass that was similar to that for the recombinant proteins. Therefore, the results support that the recombinant proteins are replicas of the native molecules produced by L. infantum. A second band of MW ˜45 kDa was also recognized by the anti-Li-nom antiserum in the crude parasite lysate. It is possible that the Li-ntf2 aggregates with itself or that the band is part of a larger protein complex or a building block of a larger protein originated by post-translational modification.
Recognition of Recombinant Li-isd1, Li-txn1, and Li-ntf1 Proteins by Sera from Patients with VL:

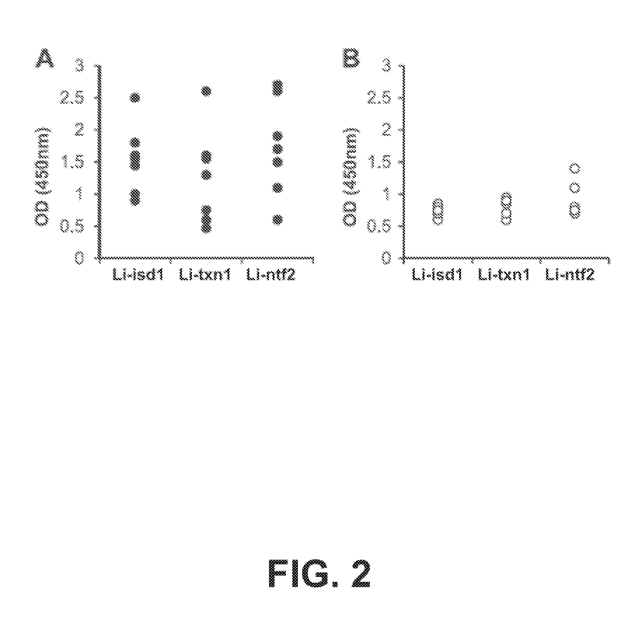

To evaluate if the discovered antigens were biologically active during disease, ELISAs were carried out using sera from seven well characterized patients with active VL. All patients were bone marrow smear-confirmed for VL. The patients and control sera were from the Natan Portella Institute of Tropical Medicine, Teresina, PI, Brazil. FIG. 2 indicates that the patients' sera tested by ELISA reacted with the three antigens at a higher titer than did control sera (n=5). These results suggest that during disease the L. infantum Li-isd1, Li-txn1, and Li-nft2 proteins are produced in sufficient quantities to sensitize the patient's immune system to produce specific antibodies, thus confirming them as biologically significant molecules produced in vivo during disease.

### Optimization of a Capture ELISA for Li-isd1, Li-txn1, and Li-ntf1:

Reagents were initially tested in order to obtain the highest sensitivity limit of detection (LOD) to identify the Leishmania antigens in human urine. Purified rabbit and chicken immunoglobulins (IgG/IgY) were initially titrated and those antibodies that provided optical density (OD) readings arbitrarily higher than 0.5 above the background at the lowest concentrations were selected for further evaluations as capture reagents. The purified antibodies were then titrated as capture reagents in an antigen detection ELISA. Plates were coated with different concentrations of the antibodies followed by incubation with a fixed concentration of corresponding antigens (5 ng/ml). Biotinylated antibody specific for each antigen was then added followed by peroxidase-labeled streptavidin and TMB. This approach led to the selection of antigen affinity-purified rabbit antibody specific for the antigen Li-txn1, a whole rabbit IgG fraction (affinity purified using Streptococcus protein G) specific for Li-ntf2, and an antigen affinity purified chicken IgY specific for the antigen Li-isd1. The concentrations of antibody required to provide the highest OD signals above the background varied for each antigen/antibody capture ELISA and were 100 ng/well, 875 ng/well, and 2,000 ng/well for Li-isd1, Li-txn1, and Li-ntf2, respectively.

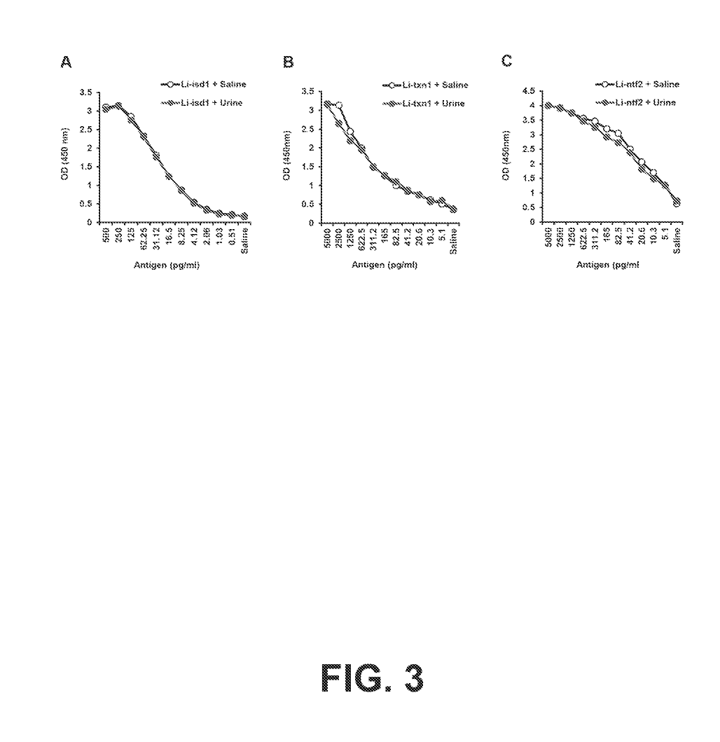

To determine the LOD, several concentrations of the antigens were tested in the presence and absence of human urine samples obtained from normal and healthy subjects. As can be seen in FIG. 3, as little as ˜4 pg/ml of the Li-isd1 antigen can be detected in the presence of either PBS or control human urine. The limits of detection for Li-txn1 and Li-ntf2 were ˜20 pg/ml and ˜5 pg/ml, respectively. Importantly, normal human urine did not interfere with the sensitivity of the antigen detection capture ELISAs specific for the three candidate antigens. These results are important in that they indicate that interference of urine should not occur with assays developed to diagnose VL using this human specimen.

### Detection of Li-isd1, Li-txn1, and Li-ntf1 In Patients' Urine Specimens Using Capture ELISA:

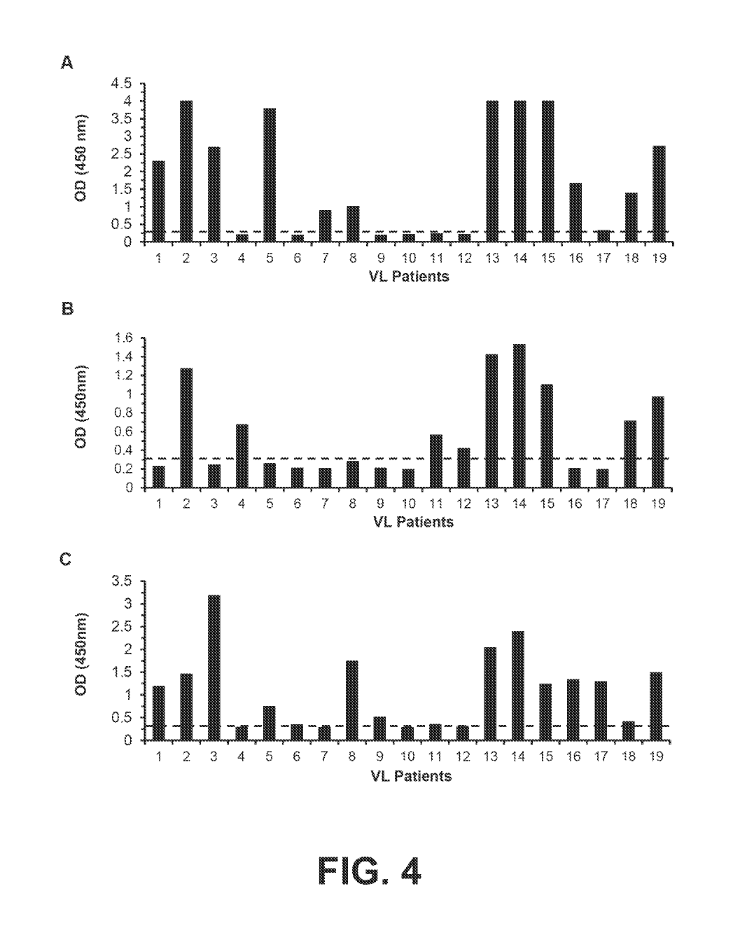

Given that the limit of antigen detection of the capture ELISAs assembled with different antibodies to the L. infantum antigens present in the urine of patients with VL was in the range of 5-50 pg/ml of protein and that human urine did not interfere with the assay sensitivity we next investigated the possible utility of these assays for the development of an antigen detection assay for the diagnosis of active VL. This type of assay is in high demand to accurately diagnose VL and to distinguish active disease from asymptomatic infection. To validate the assay, 19 urine specimens from well-characterized VL patients from the Natan Portella Institute of Tropical Medicine (Teresina, PI, Brazil) and 16 urine specimens from healthy control individuals were initially tested. The patient population comprised of 4 females aged 2-36 years old and 15 males aged 5-44 years old. Diagnosis of VL was based on the presence of the following signs and symptoms in all patients: chronic and irregular fever, abdominal pain, progressive emaciation, malaise, anemia, weight loss, hepatosplenomegaly (>2 cm from the costal margin), and finding of a leishmania amastigote in bone marrow aspirates. All urine specimens were collected before the initiation of the anti-leishmania therapy. FIG. 4 shows the results and indicates that the majority of the VL patients' urine samples provided strong positive ELISA signals for the three antigens. The cutoffs to consider positive signals were established as the OD readings given by the mean of the readings of the 16 urine samples from healthy normal individuals plus 3 standard deviations (SDs) of the mean. Specifically, 15 patients' samples provided strong positive ELISA signals for the antigen Li-isd1. In addition, an equal number of urine samples provided positive ELISA signals for the antigen Li-ntf2, and 9 patients' urine samples were positive for the antigen Li-txn1. Altogether, these results validate the use of these L. infantum proteins as important candidates for the development of a sensitive and specific antigen detection assay for the diagnosis of active VL.

### Specificity of the Capture ELISA for VL:

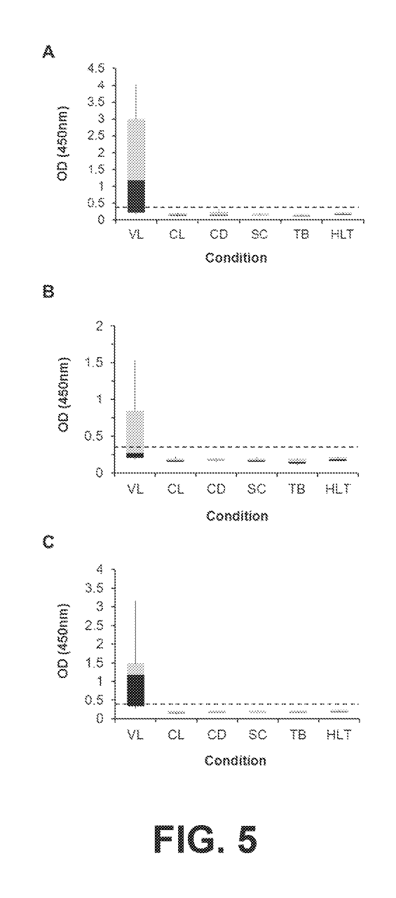

To begin to evaluate the specificity of the capture ELISA for VL, urine samples were collected from patients with cutaneous leishmaniasis (CL), Chagas' disease, schistosomiasis, and tuberculosis (TB). These diseases were chosen for this initial specificity validation because they represent important pathologies caused by organisms that produce proteins that can potentially cross react with the antibodies used in the VL antigen detection assay under study. Therefore, the capture ELISAs for Li-isd1, Li-txn1, and Li-ntf2 were performed simultaneously with the urine specimens from these patients and the urine specimens from VL patients. No cross reaction was observed with any of the urine specimens from the four groups of patients tested (FIG. 5). Although the number of patients thus far studied is limited, these results suggest that the antigen detection assay under development should be highly specific for VL.

### Discussion

The translation of this strategy for antigen discovery to patients with VL was readily achieved. Pooled urine specimens collected from patients with well-characterized VL yielded over 400 peptide sequences with homology to those of human proteins and eight peptide sequences with homology to those of three L. infantum proteins. Therefore, we reasoned that these molecules are interesting possible molecular markers of active disease and, consequently, good candidates for the development of an antigen detection assay for VL.

Expression and purification of the recombinant proteins were achieved with no major difficulties and the validation as genuine L. infantum molecules was done by Western blot analysis. The demonstration that the molecule was produced in vitro was done using rabbit or chicken antisera specific for the recombinant proteins. The antisera specific for the proteins Li-isd1 and Li-txn1 detected a single band of the expected molecular mass of L. infantum proteins in the parasite lysate. In contrast, the antiserum specific for Li-ntf2, in addition to a band that migrates at approximately the 14 kDa position, which coincides with the predicted molecular mass of the native Li-ntf2 (13.89 kDa) detects a second band that migrates at ˜45 kDa molecular mass position. At this time we cannot conclude whether the band of ˜45 kDa is a homo- or heteropolymer of Li-ntf2, or not. Nonetheless, it is unlikely that it represents a nonspecific reaction of the antiserum with an unrelated antigen present in L. infantum because the recognition of this band was much stronger than that of the band that matches the predicted molecular mass of the native molecule. However, further investigation will need to be carried out to clarify the nature of this 45 kDa molecule. Moreover, and as expected, all three recombinant molecules migrate slightly slower than the native molecules. This is indeed expected as the recombinant proteins have a slightly higher molecular mass than the native molecules due to the presence of the 6H is tag.

The concept of detecting microbial molecules in human bodily fluids of infected individuals for diagnosis purposes has strong precedent. For example, molecules from numerous viruses, bacteria such as Streptococcus pneumoniae or Legionella pneumophila, and parasites such as Entamoeba histolytica have long been described in various human samples (e.g., blood, mucous secretions, and feces) of patients suffering from the diseases caused by these microorganisms. Interestingly, many of these molecules were successfully used either as vaccines (e.g., for hepatitis A and B) or as tools for the development of antigen detection-based diagnostics. Perhaps the most successful example of such tests is the commercially available test to detect Streptococcus pyogenes (Group A) in patients with tonsillitis. This rapid test has been universally used as a routine diagnostic of S. pyogenes pharyngitis for more than 10 years.

Paradoxically, although an antigen detection assay has the potential to discriminate active VL from asymptomatic infection and cured patients, development of such a test has only recently become a matter of interest. Indeed, a test that is based on detection of L. donovani polysaccharide antigens in the urine of VL patients is under clinical validation. Despite conflicting results regarding the sensitivity and specificity a significant correlation between this assay with conventional serological and parasitological tests has been reported. Therefore, the highly purified recombinant proteins described in the present studies are of great interest as candidates to either replace or complement the sensitivity and specificity of the previously described polysaccharide detection test. This possibility is supported by the findings that together, the protein detection test can detect the native antigens in the urine of 17/19 patients with active VL. Interestingly, no cross reaction was observed with urine specimens of patients with CL, Chagas' disease, schistosomiasis, or TB, pointing to high specificity. Although the sensitivity is slightly below the ideal for an actual clinical test, we believe that the results are highly encouraging because the capture ELISA used to detect the antigen Li-txn1 and Lintf2 was assembled with whole IgG fractions from the immunized rabbits and not with antigen-purified antibody. We are currently in the process of preparing monoclonal antibodies specific for these antigens as well as large quantities of the recombinant molecule to facilitate the purification in large amounts of antibody to assemble a more sensitive capture ELISA. These reagents will also facilitate the development of other detection systems, e.g., tests based on luminescence or fluorescence readouts (Rissin D M, et al., Single-molecule enzyme-linked immunosorbent assay detects serum proteins at subfemtomolar concentrations, Nat. Biotechnol., 28: 595-599 (2010)).

It is important to mention that because antigen detection assays are highly dependent on the antigen load present in the sample under analysis they are theoretically useful tools to follow up therapy efficacy particularly of infectious diseases. Therefore, it will be interesting to test VL capture ELISAs for the antigens Li-isd1, Li-txn1, and Li-ntf2 for this purpose. In addition, the capture ELISA should be useful for the diagnosis of VL in VL/HIV-coinfected patients, as the conventional serological diagnosis of VL in these patients is problematic and not sensitive.

Finally, the genes coding for the identified L. infantum proteins are highly conserved among the organisms of the L. donovani complex. Therefore, the antibodies raised against the L. infantum recombinant proteins should recognize equally well the proteins produced by L. donovani thus suggesting that the proposed test will be equally sensitive to detect VL caused by either L. donovani or L. infantum.

In conclusion, these results strongly support the premise of the approach used in these studies, confirming that we have developed a powerful and reliable antigen discovery strategy to directly identify L. infantum diagnostic candidate antigens in human bodily secretions. In addition, these results confirm our previous hypothesis that this approach is broadly applicable to several infectious diseases, particularly those caused by organisms that have their genome already completely sequenced.

### Example 2

### L. infantum chagasi ntf1, a Vaccine Candidate for Kala-Azar

Here, we describe an innovative approach for the direct identification of VL vaccine candidate molecules that are produced in vivo during disease and that are present in bodily fluids of patients with VL. This approach led to the identification of several polypeptides of L. infantum chagasi, one of which has been extensively studied and is reported here. Importantly, this antigen, Li-ntf-2 (NCBI accession XP_{—}001463738.1), tested as a vaccine candidate formulated with the adjuvant BpMPLA-SE, induced marked parasite burden reduction in a mouse model of VL.

### Material and Methods

### Human Samples:

A total of seven urine samples were evaluated in this study. These samples were collected from seven patients diagnosed with VL based on the following criteria: a clinical course consistent with VL (e.g., fever, anemia, hepatosplenomegaly), and confirmatory laboratory findings (identification of Leishmania in bone marrow aspirates). Two patients were from the University Hospital, University of Brasilia Medical School (Brasilia, DF, Brazil) and five patients were from the Natan Portella Institute of Tropical Diseases, Federal University of Piaui, Teresina, PI, Brazil. Urine donation protocol was approved by the Investigational Review Boards and Ethics Committees of both University hospitals. Urines were frozen immediately after collection and sent frozen to The Forsyth Institute, Cambridge, Mass., USA.

### Mass Spectroscopy Analysis:

Individual samples (15 ml) were concentrated using Centricon P3 (3 kDa cutoff filters) to approximately ˜200-300 μl. Urine samples were then submitted to SDS-PAGE followed by Coomassie staining Bands were excised from the gel and submitted for mass spectroscopy analysis at the Taplin Mass Spectrometry Facility, Harvard Medical School, Boston, Mass. Gel bands were then trypsin-digested into peptides. Peptides were analyzed by nano-scale liquid chromatography coupled to a tandem mass spectrometer. Eluted peptides first had their molecular masses measured, then were fragmented, and finally the fragment masses were measured. The specific fragmentation pattern was computer-searched against predicted tryptic peptides from all known proteins from genome sequencing projects of human and Leishmania. The power of the technique is in its redundancy. Because many peptides are generated from the initial gel band, multiple matches to the protein of interest are detected. In this way, the protein identity is completely unambiguous. Peptide score cutoff values were chosen at Xcorr of 1.8 for singly charged ions, 2.5 for doubly charged ions, and 3.0 for triply charged ions, along with deltaCN values of 0.1, and RSP values of 1. The cross correlation values chosen for each peptide assure a high confidence match for the different charge states, while the deltaCN cutoff insures the uniqueness of the peptide hit. The RSP value of 1 insured that the peptide matched the top hit in the preliminary scoring and that the peptide fragment file only matched to one protein hit.

### Animals:

Female BALB/c mice and LVG Golden Syrian hamsters were purchased from Charles River Laboratories (Wilmington, Mass.) and kept under specific pathogen-free conditions. Animals were used at 12 weeks of age. All experiments were carried out under the guidelines of the Institutional Animal Care and Use Committee at the Forsyth Institute.

### Parasite:

The L. infantum chagasi strain used in these studies was kindly supplied by Dr. Mary E. Wilson (University of Iowa, Iowa City, Iowa) and was maintained in vivo in hamsters. Metacyclic promastigotes of the parasite were used for challenge infections. For challenge infection of BALB/c mice parasites were isolated from the spleen of hamster and cultured in Schneider's medium (Invitrogen, Carlsbad, Calif.) supplemented with 20% FBS (Hyclone, Thermo Scientific, Rockford, Ill.) and 2 mM L-glutamine (Gibco-Invitogen, Carlsbad, Calif.) for 7-10 days at 26° C.

Cloning of Leishmania infantum Chagasi Gene, Protein Expression and Purification:

Oligonucleotide PCR primers were designed to amplify the full-length open reading frame of the target gene from genomic DNA of L. infantum chagasi. The forward primer contained an NdeI restriction site at the ATG initiation codon followed by sequences derived from the gene. The reverse primer included a BamHI restriction site followed by a stop codon and sequences from the gene. The resultant PCR product was digested with restriction enzymes and subcloned into pET-14b expression vector, which was similarly digested for directional cloning. Alternatively, the Li-ntf-2 DNA sequence was codon optimized for expression in E. coli containing the same restriction enzyme sites (NdeI and BamHI). The DNA fragment was synthesized (Blue Heron, Bothel, Wash.) and the synthetic gene was sub-cloned into pET-14b as well. A ligated pET-14b vector was subsequently used to transform E. coli BL21(DE3)pLysS host cells (Novagen, Madison, Wis.) for expression. Recombinant protein was obtained and purified from 100 mL of IPTG-induced batch cultures by affinity chromatography using QIAexpress Ni-NTA agarose matrix (QIAGEN, Chatsworth, Calif.) as described. The yields of recombinant protein were 10-20 mg per liter of induced bacterial culture, and purity was assessed by SDS-PAGE followed by Coomassie blue staining Endotoxin level present in the purified recombinant protein preparations was measured by the Limulus Amebocyte Lysate assay (Lonza, Walkersville, Md.) and shown to be 2.89 EU/ml.

### Western Blot:

Purified recombinant proteins (100 ng) and whole antigen extract from Leishmania infantum chagasi were fractionated by SDS-PAGE (4-20% gradient gel) and transferred to polyvinylidene fluoride membrane (PVDF, Millipore, Medford, Mass.). Crude Leishmania chagasi lysates were prepared from promastigote parasites cultured for 7-10 days in complete Schneider's medium at 26° C. The blots were blocked overnight at 4° C. with Tris-buffered saline with 0.1% Tween 20 (TBS-T) containing 1% bovine serum albumin (BSA) and subsequently probed with antigen-specific rabbit antisera and pre-immune rabbit. After several rinses with TBS-T, goat anti-rabbit IgG labeled with horseradish peroxidase (ThermoScientific Pierce, Rockford, Ill.) was added. After additional washings, bound conjugates were detected using ECL enhanced chemiluminescence system (Amersham/GE Healthcare, Piscataway, N.J.) and proteins were visualized by autoradiography (Kodak BioMax, Rochester, N.Y.).

### Immunization Regimen and Challenge:

Groups of 10 mice were injected subcutaneously as follows: control mice were injected with saline plus the adjuvant BpMPLA-SE (20 μg/ml) and immunized mice were injected with recombinant protein (5 μg/mouse) in presence of 20 μg/ml of the adjuvant BpMPLA-SE (Institute Butantan, Sao Paulo, Brazil). BpMPLA-SE is an oil-in water emulsion containing monophosphoryl lipid A (MPLA) derived from Bordetella pertussis. The B. pertussis MPLA_SE is a stable oil-in-water emulsion that contains squalene (the oil phase) and the surfactant Tween 80. A detailed description of this adjuvant formulation and its biological and immunological properties has been described elsewhere (Miyaki C, Quintilio W, Miyaji E N, et al. Production of H5N1 (NIBRG-14) inactivated whole virus and split virion influenza vaccines and analysis of immunogenicity in mice using different adjuvant formulations. Vaccine 2010; 28: 2505-2509; Quintilio W, Kubrusly F S, Iourtov D, et al. Bordetella pertussis monophosphoryl lipid A as adjuvant for inactivated split virion influenza vaccine in mice. Vaccine 2009; 27: 4219-4224). Mice in each group were vaccinated three times with same doses of inoculums following three weeks intervals. Ten days after the last immunization three mice from each group were euthanized to carry out T cell immunogenicity assays, and four weeks after immunization remaining mice were challenged intravenously with 10×10⁶ L. infantum chagasi promastigotes to assess vaccination efficacy.

### In Vitro Cytokine Response Assay:

Ten days after the last boost spleens were excised and splenocytes harvested using 70 μm cell strainers. After centrifugation over Histopaque (Sigma, St. Louis, Mo., USA) and washings, mononuclear cell suspensions were prepared in RPMI supplemented with 10% FBS (Hyclone), 100 μg/mL streptomycin, 100 U/mL penicillin, 25 mM HEPES, 2 mM L-glutamine, 0.05 mM 2-ME (all Sigma). Cell viability was estimated with Trypan Blue 0.4% (Sigma). About 2×10⁵ cells were added to the wells of a 96-well flat-bottomed culture microplate (Costar, Lowell, Mass., USA). Cells were stimulated for 72 h with 5 μg/mL of recombinant protein. Supernatants were collected and frozen until use. Cells cultured in the presence of ConA (5 ng/mL) or complete medium alone were included as controls. Cytokine (IFN-γ and IL-4) concentration in the supernatants was measured using specific sandwich ELISA kit (R&D Systems, Minneapolis, Minn., USA).

### Flow Cytometry and Intracellular Cytokine Staining Assay:

Ten days after the last boost mice were sacrificed, spleens were excised, and splenocytes were obtained as described earlier. Cells from both immunized and control mice were counted and adjust to 2×10⁷ cells/ml in RPMI 1640 medium (Gibco-Invitrogen) supplemented with 10% FBS, 25 mM HEPES, 2 mM L-glutamine, 20 U of penicillin/ml, 20 μg of streptomycin/ml. Two million cells (100 μl) from individual mice were incubated with medium or recombinant protein (2 μg/ml) plus Golgi Stop (2 μl/ml) on a 96-cell culture plate. As positive control, splenocytes were incubated with PMA (2 μg/ml), ionomycin (10 μg/ml) and Golgi Stop. The cells were incubated at 37° C. for 6 h and transferred to 4° C. overnight. The following day, cells were washed with 2% FBS in PBS followed by incubation with monoclonal antibodies specific for cell surface molecules for an additional 30 min. Permeabilization was performed for one hour with Cytofix/Cytoperm solution (BD Biosciences, San Jose, Calif.). Cells were washed with 1× Perm/Wash buffer (BD Biosciences) and then stained with anti-cytokine mAb (anti-IFN-γ or anti IL-4). After an additional washing with 1× Perm/Wash buffer, the cells were fixed in 2% formaldehyde-PBS. Samples were collected on FACSAria III instrument (BD Biosciences, San Jose, Calif.) and analyzed using FlowJo software (Tree Star, Ashland, Oreg.). The following antibodies from BD Biosciences were used: anti-CD3e-FITC (145-2C11); anti-IFN-γ-APC (XMG1.2); anti-IL-4-PE (BVD4-1D11); and anti-CD4-APC-Cy7 (GK1.5).

### Parasite Quantification:

The load of Leishmania in the spleen was estimated by limiting dilution assay. Forty days after challenge with L. infantum chagasi promastigotes animals were euthanized and spleens were removed and homogenized in complete Schneider's medium. Homogenates were 10-fold serially diluted in 12 replicates for each dilution in 96-well flat-bottomed microplates (one plate per spleen) and incubated for 7-10 days at 26° C. The number of viable parasites was calculated from the highest dilution in which parasites could be found under phase contrast light microscopy in any of the 12 replicates (Titus R G, Marchand M, Boon T & Louis J A. A limiting dilution assay for quantifying Leishmania major in tissues of infected mice. Parasite Immunol 1985; 7: 545-555).

### Antibody ELISA:

IgG1 and IgG2a antibodies raised against L. infantum chagasi recombinant protein were titrated by standard ELISA protocol. High binding 96-well microplates (Costar) were coated with purified recombinant protein (2 μg/ml) prepared in 0.2M sodium carbonate-bicarbonate buffer (pH 9.6) and incubated overnight at 4° C. Wells were washed with PBS containing 0.05% Tween 20 (PBS-T) and blocked with 1% BSA in PBS-T for 2 hours. Serum samples were added at 2-fold serial dilutions in PBS-T containing 0.1% BSA and plates were incubated for 1 hour at RT. After another washing step, biotinylated rat anti-mouse IgG1 or IgG2a mAb (2 μg/ml, BD Biosciences) was added and incubated for 1 hour. Streptavidin-HRP conjugate (BD Biosciences) was used at 1/2000 followed by addition of ready-to-use substrate solution containing tetramethylbenzidine (KPL, Gaithersburg, Md.). Color development was stopped using 1N HCl. Optical density data were recorded at A₄₅₀.

### Statistical Analysis:

Data are presented as mean and SEM. Statistical significance was determined by the Student's t test for comparison of two groups. Values of p<0.05 were considered statistically significant.

### Results

### Antigen Discovery of L. Infantum Chagasi Proteins in Urine of VL Patients:

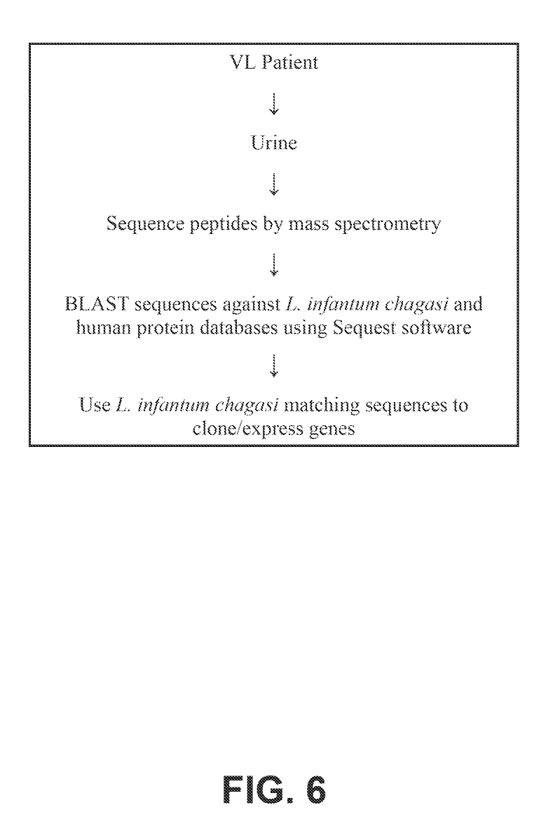

The general protocol to analyze the human urine samples from VL patients is illustrated in FIG. 6. Urines were initially collected from seven patients with active, culture-confirmed VL (University Hospital, University of Brasilia, Brasilia, DF, and Federal University of Piaui, Teresina, PI, Brazil). These patients did not have any clinical signs or symptoms or laboratory findings compatible with renal or urinary tract abnormalities. None of the patients were under anti-leishmaniasis therapy at the time of urine collection. Individual urine samples were analyzed by mass spectrometry and generated approximately 400 peptide sequences. As expected, most sequences of the identified peptides had identical sequence homologies with that of human proteins. Importantly, eight peptide sequences that had no known homologies with human proteins had identical sequence homologies with the deduced sequences of three different L. infantum chagasi proteins. Two of these peptides (LNAAAESNSGLASK (SEQ ID NO:2) and GGGEPSGPLASAIVDSFGSFASFK (SEQ ID NO:4)) had highly significant XCorr (3.602 and 5.274 respectively) and perfect matching sequences with the sequence of Leishmania infantum chagasi iron superoxide dismutase 1 (NCBI accession: XP_{—}001467866.1; Gene ID EuPathDB: LinJ.32.1910) at positions 40-53 and 87-110, respectively. Four of other peptides (QNDMVDMSSLSGK (SEQ ID NO:6), MPWLSIPFEK (SEQ ID NO:8), QYKVESIPTLIGLNADTGDTVTTR (SEQ ID NO:10) and VESIPTLIGLNADTGDTVTTR(SEQ ID NO:12)) had highly significant XCorr (2.813, 2.789, 3.63, and 4.168, respectively) and perfect matching sequences with the sequence of Leishmania infantum chagasi tryparedoxin 1 (NCBI accession: XP_{—}001466642.1; Gene ID EuPathDB: LinJ.29.1250) at positions 17-29, 84-93, 103-126, and 106-126 respectively. Finally, two peptides (FANLGFTEAAFK (SEQ ID NO:14) and EQVQGVDAIMAR(SEQ ID NO:16)) had highly significant XCorr (4.0849 and 3.1871, respectively) and perfect and strong matching sequences with the sequence of a putative L. infantum chagasi protein (nuclear transport factor 2, NCBI accession XP_{—}001463738.1; Gene ID EuPathDB: LinJ.10.0900) at positions 40-51 and 52-63, respectively. Importantly, these L. infantum chagasi peptides were identified in multiple urine samples, thus strongly validating these findings. FIG. 7 depicts the discovered proteins and highlights the peptides found in the patients' urines. Overall, these findings suggest that these proteins are abundant antigens of L. infantum chagasi produced during the disease and broadly present in human bodily fluids.

Both L. infantum chagasi iron superoxide dismutase 1 and L. infantum chagasi tryparedoxin 1 have been previously described and their immunological properties studied. In contrast, little is known about the biology of the putative L. infantum nuclear transport factor 2 (Li-ntf2), and no reports were found about the immunological significance of the molecule. Therefore, the present study concentrates on the evaluation of Li-ntf2 as a vaccine candidate for VL.

The open reading frame of the full-length gene coding Li-ntf2 was amplified by PCR and sub-cloned into the E. coli pET-14b expression vector. This cloning strategy resulted in no expression of protein. However, using a synthetic gene with optimized sequence for E. coli followed by sub-cloning in pET-14b resulted in excellent over-expression of the recombinant protein, which was then purified using the Ni-NTA agarose resin.

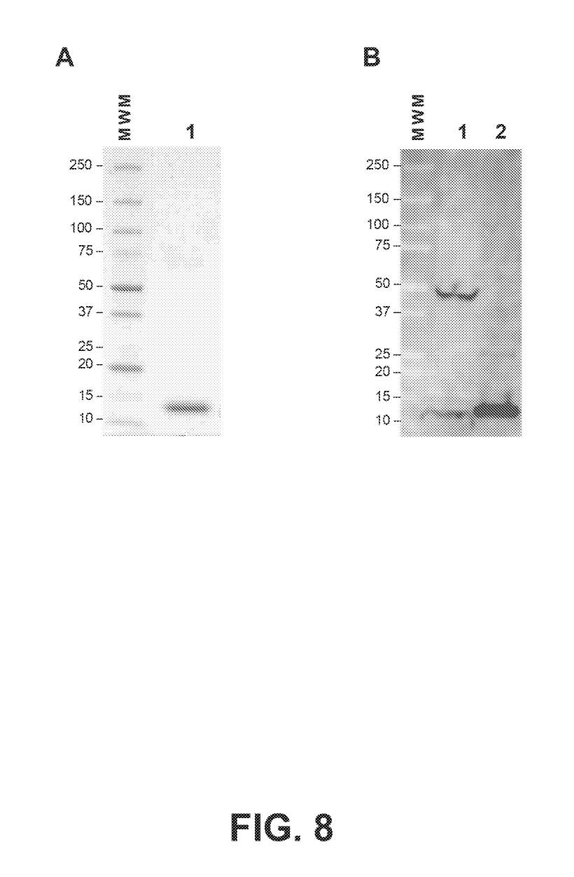

In order to validate the E. coli-expressed and purified protein (FIG. 8A) as true L. infantum chagasi protein, a crude lysate of the parasite and the recombinant protein were immunoblotted and then probed with specific polyclonal rabbit anti-recombinant Li-ntf2 antisera (FIG. 8B). The antiserum clearly recognized a protein band in the crude parasite lysate at similar molecular weight as the recombinant protein. Therefore, the results support that the recombinant Li-ntf2 is a replica of the native protein produced by L. infantum chagasi. A second band of MW ˜45 kDa was also recognized by the antiserum. However, the nature of this band is not known. It is possible that the Li-ntf2 aggregates with itself, or is part of a larger protein complex, or a building block of a larger protein originated by post-translational modification.

### Immunogenicity Studies in Mice:

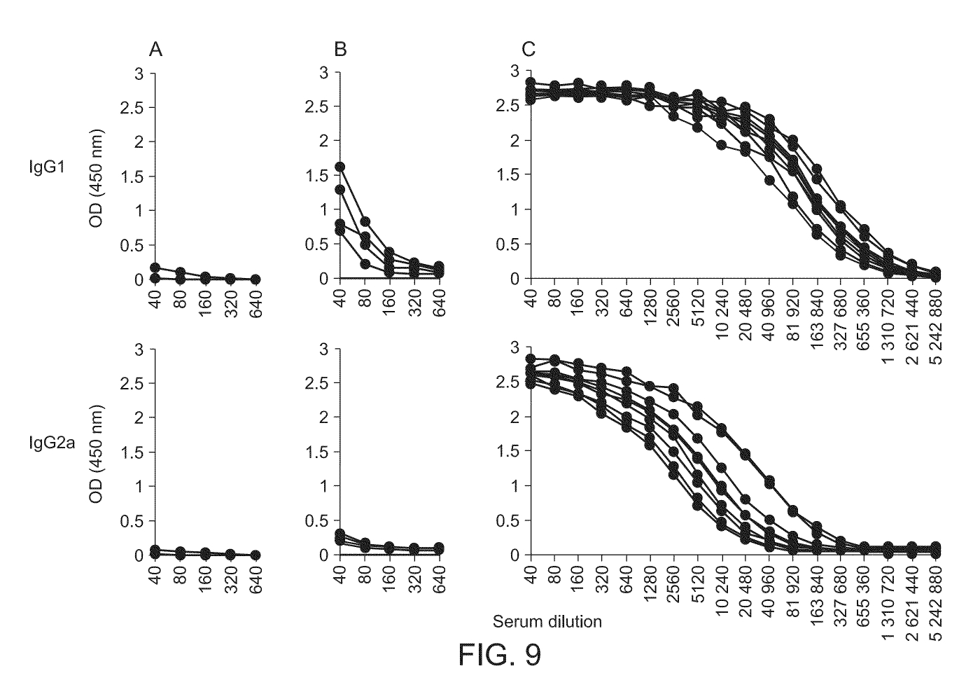

Because CD4+ T cells of the Th1 phenotype are crucial mediators of resistance to VL, we chose to immunize mice with a formulation containing the purified recombinant Li-ntf2 plus the adjuvant monophosphoryl lipid A (MPLA) from Bordetella pertussis emulsified with squalene (Butantan Institute, São Paulo, SP, Brazil). An MPL-based adjuvant was chosen because this type of formulation has been shown to be safe and to preferentially stimulate Th1 responses. Protection against cutaneous leishmaniasis induced by recombinant antigens in murine and nonhuman primate models of the human disease. A clinical trial to evaluate the safety and immunogenicity of the LEISH-F1+MPL-SE vaccine when used in combination with meglumine antimoniate for the treatment of cutaneous leishmaniasis. Vaccine 2010; 28: 6581-6587; Miyaki C, Quintilio W, Miyaji E N, et al. Production of H5N1 (NIBRG-14) inactivated whole virus and split virion influenza vaccines and analysis of immunogenicity in mice using different adjuvant formulations. Vaccine 2010; 28: 2505-2509; Quintilio W, Kubrusly F S, Iourtov D, et al. Bordetella pertussis monophosphoryl lipid A as adjuvant for inactivated split virion influenza vaccine in mice. Vaccine 2009; 27: 4219-4224). Squalene is added to condition a stable oil-in-water emulsion (BpMPLA-SE) to further increase the stimulation of the immune system. Mice were immunized three times with 5 μg of Li-ntf2 alone or mixed with 20 μg of BpMPLA-SE. As a control, another group of mice was immunized with saline+BpMPLA-SE. The choice of 20 μg of BpMPLA-SE was based on initial dose-response studies that indicated that this concentration, compared to 2.5, 5.0, and 10 μg, induced the most robust immune response (not shown). Ten days after the last immunization mice were bled (for serologic studies) and sacrificed. Spleens were removed and splenic cell suspensions were prepared and stimulated for 6 hours with either medium (control) or purified recombinant Li-ntf2. Cells were harvested and cytokine responses (IFN-γ and IL-4) were measured by flow cytometry for intracellular cytokine staining (ICS). Culture supernatants from cells stimulated for 72 h were used to measure total cytokine response by conventional capture ELISA. FIG. 9 shows the serological titration of the IgG1 and IgG2a specific antibody response to the recombinant Li-ntf2. High titers of both IgG1 and IgG2a were generated after immunization with Li-ntf2+BpMPLA-SE. In contrast, animals immunized with the protein alone (mixed with saline) produced low titers of IgG1 and no detectable levels of IgG2a. Because class switch to IgG2a is dependent on the production of IFN-γ, this result is consistent with a stimulation of Th1 response by the antigen/adjuvant formulation.

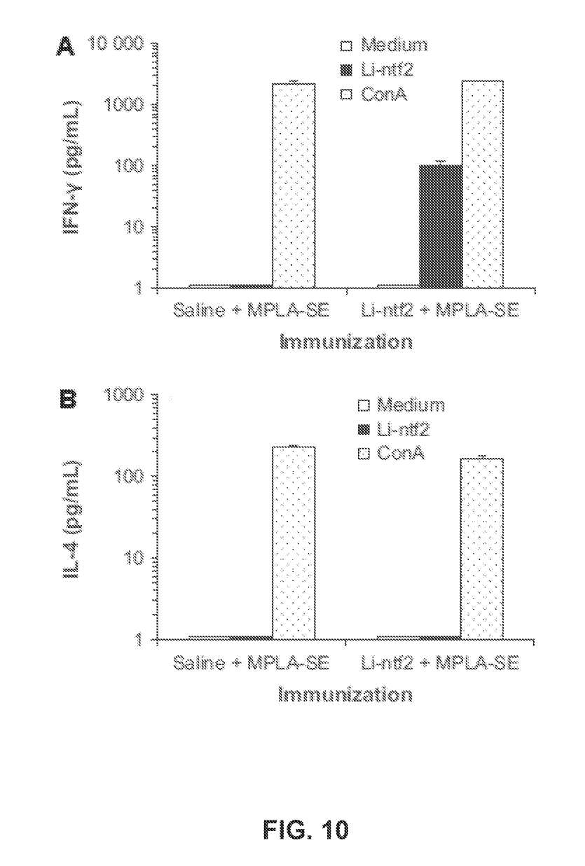

The antigen stimulated cytokine response of cells obtained from animals immunized with Li-ntf2-BpMPLA-SE as well as with saline+BpMPLA-SE was initially tested in the culture supernatant of spleen cell cultures stimulated with Li-ntf2. Results are illustrated in FIG. 10. In the cell cultures obtained from antigen immunized mice and stimulated in vitro with Li-ntf2, the Th1 cytokine IFN-γ was readily detected in the culture supernatants. This cytokine was not detected in the culture supernatants of cells stimulated with medium alone or in the cultures from spleen cells obtained from mice immunized with saline+BpMPLA-SE and stimulated in vitro with recombinant Li-ntf2. Moreover, no IL-4 could be detected in the supernatants of antigen-stimulated cultures. However, both IFN-γ and IL-4 were readily detected in the culture supernatants of cultures stimulated with the mitogen ConA thus confirming the potential of the cells to produce both cytokines. These results confirm the antigen specificity of the responses obtained from cells of mice immunized with recombinant Li-ntf2 plus BpMPLA-SE, i.e., the trace amount of LPS (<2.9 EU/ml) present in the antigen preparation is not responsible for the antigen induction of cytokine production by cells from immunized Li-ntf2-immunized mice.

The confirmation that the IFN-γ detected in the culture supernatants was produced by CD4+ T cells was obtained by intracellular cytokine staining (ICS). The results are illustrated in FIG. 11. As can be seen, the results obtained using ICS clearly point to CD4+ T cells as an important source of IFN-γ produced upon stimulation with recombinant Li-ntf2. In the cell cultures obtained from antigen immunized mice and stimulated in vitro with Li-ntf2, the Th1 cytokine IFN-γ was readily detected (FIGS. 11A, B upper panel). In contrast, no IL-4-producing cells could be detected (FIGS. 11A, B, lower panel). As expected, no IFN-γ- or IL-4-producing cells were detected in cells obtained from mice immunized with saline+BpMPLA-SE and re-stimulated in vitro with Li-ntf2. However, CD4+ T cells producing both IFN-γ and IL-4 were clearly detected in cultures stimulated with the polyclonal activators PMA+ionomicin (FIG. 11C). Together these results confirm that the immunization protocol using the adjuvant BpMPLA-SE induces a preferential Th1 response to the L. infantum chagasi vaccine candidate Li-ntf2.

### Protection Studies in Mice:

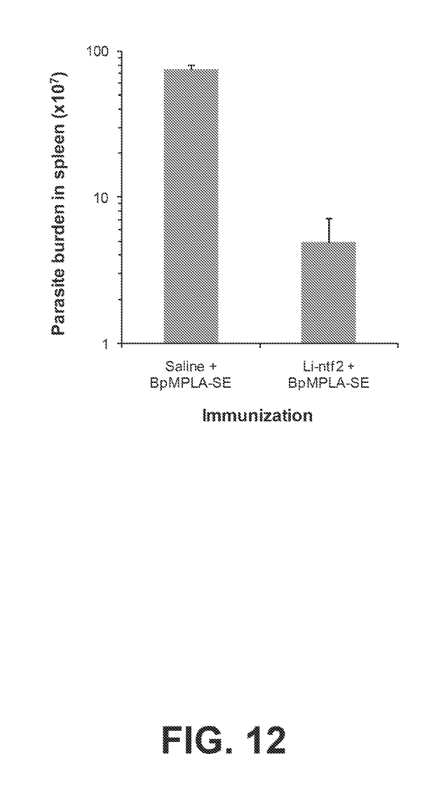

Mice have been used and accepted for initial evaluation and selection of vaccine candidate antigens for future validation in large animal models (e.g., dogs). Two groups of BALB/c mice were immunized three times (three weeks interval) with either 5 μg of the recombinant Li-ntf2 mixed with 20 μg of the adjuvant BpMPLA-SE or with saline mixed with 20 μg of BpMPLA-SE. For these experiments, a group of animals immunized with Li-ntf2 alone was not included because the immunogenicity studies indicated that this formulation stimulated little or undetectable immune response. Thirty days after the last immunization mice were challenged i.v. with 10×10⁶ promastigotes (stationary phase of growth) freshly derived from amastigotes isolated from the spleen of L. infantum chagasi-infected hamster. To determine protection, animals were sacrificed 40 days later and spleen homogenates were prepared in a serial dilution manner and incubated in Schneider's medium for limiting dilution quantification. Parasite enumeration was performed one week later and results are expressed in FIG. 12. As can be seen, immunization with recombinant Li-ntf2 plus BpMPLA-SE caused significant reduction in the parasite burden (1.3 log₁₀) compared to animals immunized with saline+BpMPLA-SE. Therefore, these results support the hypothesis that abundant L. infantum chagasi antigens present in bodily fluids of humans during disease can be potential target vaccine candidates.

### Discussion

Although much effort has been placed on the development of anti-VL human vaccine, little or no success has been achieved. Two vaccines to canine visceral leishmaniasis (CVL) are commercially available in Brazil but these products have not been approved for human use due to low protection efficacy in dogs as well as complications with standard operating procedures for the manufacture of one of them, which is a glycoprotein fraction purified from L. donovani promastigotes.

The barriers to achieve better success in vaccine development to VL are not entirely understood. However, most of the antigen discovery approaches tested so far have used antibodies and in some cases PBMC from patients with VL to identify parasite molecules of interest. Although these approaches have been validated for other diseases, including CL, we believe that they are not ideal for VL because VL patients have both massive hypergammaglobulinemia due to polyclonal activation of B cells and severe immunossupression of T cell responses. Consequently, these readouts do not necessarily reflect host resistance to L. donovani/infantum chagasi.

In this work we address this critical barrier of antigen discovery. Our target vaccine candidates are parasite molecules that are abundantly produced in vivo in human patients with active VL. The hypothesis of this proposal is that microbial antigens found in vivo in the bodily fluids of the host are potentially effective vaccine candidate molecules because they represent microbial molecules actively and continuously produced and released during disease, therefore by definition, good targets for vaccine development. Moreover, our former studies in tuberculosis confirm this hypothesis.

Thus, we combined RP-HPLC and mass spectrometry (MS) and categorized three distinct L. infantum chagasi proteins present in the urine of seven VL patients. The rationale to use urine as source of the pathogen's antigens was based on the premise that L. infantum chagasi proteins or their breakdown products (peptides) produced in vivo, e.g., in spleen/liver/bone marrow lesions, would reach the host's blood circulation and subsequently would be excreted in the patient's urine. The three L. infantum chagasi proteins identified in the urines of seven patients with VL were as follows: L. infantum chagasi iron superoxide dismutase, L. infantum chagasi tryparedoxin, and L. infantum chagasi nuclear transport factor 2 (Li-ntf2). The MS results were unambiguously conclusive that the peptides identified in the patients' urines were indeed derived from the parasite's proteins. This assertion is strongly supported by the facts that the inferred amino acid sequences of the derived peptides had very high XCorr (>3.0) and several different peptides found in the patients' urines spanned at different sequence positions of the corresponding peptide donor proteins.

The concept of detecting microbial molecules in human bodily fluids of infected individuals particularly for diagnosis purposes has strong precedent. For example, molecules from numerous viruses (e.g., hepatitis A and B), bacteria (e.g., Streptococcus pneumonia, Streptococcus pyogenes, Legionella pneumophila), and parasites (e.g., Entamoeba histolytica) have long been described in various human samples (e.g., blood, mucous secretions, and feces) of patients suffering from the diseases caused by these microorganisms. Comparison of serologic tests with urinary antigen detection for diagnosis of legionnaires' disease in patients with community-acquired pneumonia. Clinical utility of urinary antigen detection for diagnosis of community-acquired, travel-associated, and nosocomial legionnaires' disease. Preliminary observations using a multi-layer ELISA method for the detection of Entamoeba histolytica trophozoite antigens in stool samples. A solid-phase sandwich radioimmunoassay for Entamoeba histolytica proteins and the detection of circulating antigens in amoebiasis. Interestingly, some of these molecules have been successfully used as vaccines in humans (e.g., for hepatitis A and B). Regarding leishmaniasis, L. donovani polysaccharide antigens are present in the urine of VL patients and have been used in an antigen detection assay for the diagnosis of this disease. However, because of the polysaccharide nature of these molecules they are, by definition, not rational antigens to be used as vaccine candidates to VL. Resistance to VL is T cell mediated and it is well known that polysaccharide antigens, in contrast to proteins, by in large do not induce T cell responses. Therefore, the proteins described in the present studies can be of great interest as vaccine candidates to VL.

The gene coding for the identified L. infantum chagasi proteins are also present, in various other members of the Trypanosomatidae family including L. donovani, L. major, L. brasiliensis, L. amazonensis, African trypanosomes, and Trypanosoma cruzi.

The present studies were concentrated on the putative Li-ntf2 because little is known about the biological and immunological properties of this leishmanial molecule. Cloning of the gene and production and purification of the recombinant molecule were easily achieved. Approximately 20 mg of purified protein was obtained per liter of induced E. coli culture broth. Importantly, Western blot analyses using specific rabbit antiserum raised against the purified recombinant Li-ntf2 clearly showed that this molecule is constitutively produced by L. infantum chagasi. Two clear bands present in the parasite's whole antigen preparation could be detected by the antiserum. One band that migrates at approximately 14 kDa position coincides with the predicted MW of the native Li-nom (13.89 kDa). A second band migrates at ˜45 kDa MW position. As expected, the recombinant Li-ntf2 migrates slightly slower than the native molecule, which is due to its slightly higher MW due to the presence of the 6 His tag. It is interesting to note that the 45 kDa band reacts stronger with the specific anti-Li-ntf2 antiserum than the band that matches the predicted MW of the native molecule. The band of ˜45 kDa can be a homo- or a heteropolymer of Li-ntf2.

To verify the protection potential of Li-n we chose to formulate this antigen with a monophosphoryl lipid A (MPL) adjuvant. This choice was based in our former and successful experience with this type of adjuvant in vaccine development to cutaneous leishmaniasis. MPL has proven to modulate the immune system to a predominant Th1 response, which is essential to control leishmaniasis and several other infectious processes. The MPL used in the present studies (BpMPLA-SE) is the monophosphoryl lipid A (MPLA) derived from Bordetella pertussis lipopolysaccharide containing squalene and one surfactant that facilitates the formation of a stable oil-in-water emulsion. This adjuvant has proven to induce potent immune response to influenza virus (including H5N1) vaccine candidates. In the present studies we confirmed that this adjuvant formulated with recombinant Li-ntf2 induces in BALB/c mice a potent Th1 response to this protein.

This conclusion was reached based on several evidences. First, high titer of specific antibody response of both IgG1 and IgG2a isotypes was generated against Li-ntf2. This pattern of high level of IgG2a production is generally accepted as reliable surrogate of Th1 response. Because IL-4 is known for many years to promote immunoglobulin class switching to IgG1, an antibody response that included this isotype of immunoglobulin was believed in the past to be a surrogate of the Th2 response. However, later evidence demonstrated that IgG1 antibodies are divided into two distinct sub-families of molecules (Faquim-Mauro E L, Coffman R L, Abrahamsohn I A & Macedo M S. Cutting edge: mouse IgG1 antibodies comprise two functionally distinct types that are differentially regulated by IL-4 and IL-12. J. Immunol. 1999; 163: 3572-3576). One that is dependent on IL-4 (Th2 associated) and another that is dependent on IL-12 and IFN-γ (Th1 associated). Therefore, the generation of high titers of IgG1 antibody can only be interpreted as a surrogate of a polarized Th2 response in the absence of IgG2a antibody. In contrast, because the class switch to IgG2a is solely dependent on IFN-γ, high titers IgG2a antibody, whether or not associated with IgG1 antibody has been generally accepted as a good surrogate of a typical Th1 response. Second, in vitro antigen stimulation of cells obtained from mice immunized with Li-ntf2+BpMPLA-SE clearly indicated that IFN-γ was readily produced by CD4+ T cells. In contrast IL-4 producing CD4+ T cells could not be detected. Together, these findings clearly point to a typical Th1 phenotype of immune response induced by the antigen/adjuvant formulation.

The protection experiments in the mouse model were carried out using an identical immunization protocol used for the immunogenicity studies. Although the mice were challenged i.v. using a high inoculum (10×10⁶ promastigote forms of the parasite at stationary phase of growth) of L. infantum chagasi freshly derived from amastigote forms isolated from infected hamsters, excellent protection was achieved. Over 1.3 log reduction in parasite burden was observed in vaccinated animals compared to controls. This level of protection is comparable and even superior to most VL vaccine reported to date. The reasons to explain the difficulty to elicit a more complete protection in this animal model are not known. One possibility to explain this limitation is that most regimens of immunization do not necessarily generate CD8+ T cell responses to parasite antigens, and these cells seem to also participate in immunity/memory mechanisms of protection against Leishmania. Indeed this possibility has been evaluated in more details in infections caused by other pathogens such as Mycobacterium tuberculosis and Plasmodium. Interestingly, using heterologous prime/boost protocols of immunizations that generate both CD4+ and CD8+ T cell responses to the vaccine candidate, better protection has been achieved than with immunizations that used conventional boosting strategies. In a typical protocol, animals are primed with a vaccine candidate using an antigenic formulation that induces preferential CD4+ T cells (e.g., protein plus adjuvant) and are boosted using a formulation that stimulates preferentially CD8+ T cells (e.g., genetic immunizations using virus or plasmid DNA carriers of the vaccine candidate). Experiments designed to evaluate the efficacy of this strategy to improve the protection induced by Li-ntf2 are in progress.

It is important to emphasize that the findings reported in this work strongly support the proposed antigen discovery strategy of vaccine candidates to VL. Moreover, and equally important this simple but innovative approach of antigen discovery opens novel and broader possibilities for vaccine development to other serious infectious diseases like tuberculosis, trypanosomiasis, malaria, and AIDS.

### Example 3

### Antigen Detection Assay for Visceral Leishmaniasis

Visceral leishmaniasis (VL), or kala-azar, remains a major infectious cause of morbidity and mortality worldwide. Diagnosis of VL is performed either with obsolete, invasive, and inadequate tests or with conventional serological tests that cannot distinguish active disease from asymptomatic VL or from cured infection. Here, we describe a novel antigen detection assay for accurate diagnosis of VL using patients' urine samples. The test comprises a single antigen capture ELISA assembled with combined antibodies to the Leishmania infantum antigens Li-isd1, Li-txn1, and Li-ntf2. In this pilot study performed with 20 urine samples from well-characterized VL patients, 20 samples from healthy control subjects, and 42 samples from non-VL patients (cutaneous leishmaniasis, Chagas' disease, schistosomiasis, and tuberculosis) the sensitivity and specificity of the test were both 100%. The results are very promising and warrant a future clinical trial to validate this important assay for the diagnosis of active VL.

### Development and Validation of a New Urine Test for Active VL

An ideal VL diagnostic should: a) be capable to differentiate active disease from formerly infected but healthy individuals, b) be amenable to monitor treatment efficacy, and d) identify relapse or re-infection that is common in HIV co-infected subjects.

Recently, an innovative approach was introduced for directly identifying antigens (proteins) from Leishmania infantum (etiological agent of VL) produced in vivo, in patients with active VL. By combining reverse-phase high-performance liquid chromatography (RP-HPLC) with mass spectroscopy we identified in the patients' urines three distinct L. infantum proteins namely L. infantum iron superoxide dismutase 1, NCBI accession number XP_{—}001467866.1; L. infantum tryparedoxin 1, NCBI accession number XP_{—}001466642.1; and L. infantum nuclear transport factor 2, NCBI accession number XP_{—}001463738.1). The genes coding for these proteins were cloned, the recombinant proteins were produced and purified, and antibodies to them were produced in rabbits and chickens. The antibodies were purified from the sera and were used to develop capture enzyme linked immunosorbent assays (ELISA) designed to detect individually each of these L. infantum antigens in the urine of VL patients. In a sample of 20 irrefutably diagnosed VL patients, each L. infantum protein was identified in approximately 10-12 overlapping and non-overlapping urine samples. Moreover, in one sample no leishmanial antigen could be identified by any of the three assays. Moreover, none of the antigens was detected in patients' urines with cutaneous leishmaniasis (CL), Chagas disease (CD), schistosomiasis (SC), or tuberculosis (TB). When compiled together, the urinary antigen detection ELISAs had a sensitivity of 89%, specificity of 100%, and a limit of detection of 4-10 pg of antigen per ml of urine. These samples were from patients from the area Teresina, PI, Brazil and of Montes Claros city, MG, Brazil. Urine donation protocols were approved by the Investigational Review Board and the Ethics Committee of the Federal University of Piaui Medical School and Federal University of Minas Gerais, respectively.

Here, the results of a single assay assembled with the combination of the reagents used to individually detect the three Leishmania antigens (Li-isd1, Li-txn1 and Li-ntf2) is reported. This assay adds simplicity and sensitivity to the VL diagnostic test. As can be seen on FIG. 14, confirming our previous results, 12/20 urine samples from VL patients were positive for the antigen Li-isd1 with a cut off (dotted line) of 0.432 calculated using the average of the ODs obtained from the urine specimens from 20 normal, healthy control subjects plus 3 SDs (FIG. 14A); 9/20 VL patient urine samples were positive for the antigen Li-txn1 with a cut off of 0.610; 11/20 VL patient urine samples were positive for Li-ntf2 with a cut off of 0.647. More importantly and surprisingly, 20/20 VL patient urine samples were positive for the combined capture ELISA assay. Interestingly, this result does not simply represent the detection of the individual antigens simultaneously in the combined assay. Thus, the urine of VL patient #11 was negative for each of the three individual assays but is clearly above cut off for the combined assay. In addition, samples 6, 8, and 9, which were just borderline positive for antigen Li-ntf2, were clearly positive in the combined assay. Therefore, a synergistic effect was encountered by combining the three Leishmania antigen assays in a single capture ELISA. In summary, the combined assay was 100% sensitive in this pilot study using urine samples from patients diagnosed with VL using the gold standard test of bone marrow aspiration and parasite identification.

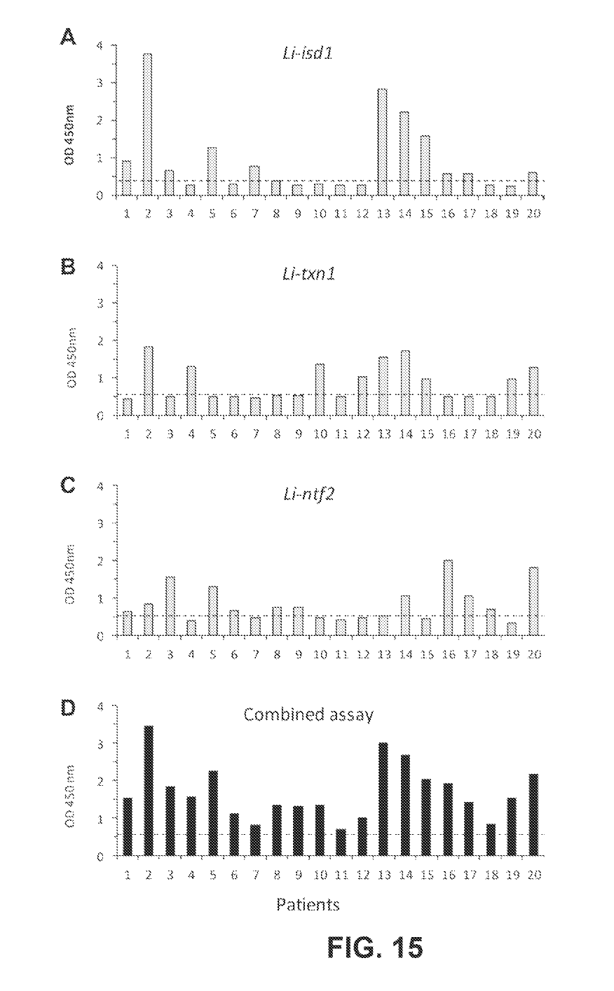

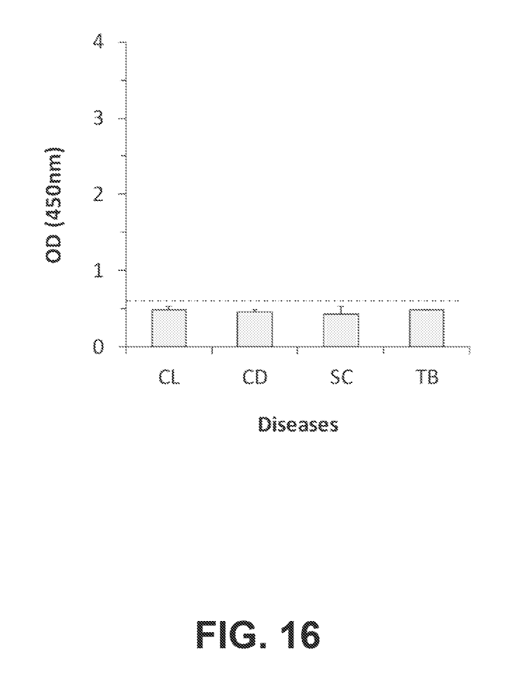

Next, the specificity of the combined capture ELISA remained at 100% was determined. Urine from patients with CL, CD, SC, and TB were tested with the combined capture ELISA and as expected were all negative (FIG. 15) keeping the specificity of the assay at 100%. This is an important observation, as specificity of a clinically useful test for VL is critical because in areas endemic for VL, diseases like CL, CD, SC, and TB are prevalent as well.

A latex agglutination test that detects leishmanial carbohydrate antigens in the urine of VL patients, KATEX™ latex agglutination test, became commercially available in Europe (Kalon Biological Ltd, Guilford, UK). However, recent field studies with KATEX™ latex agglutination test reported a wide variability in its sensitivity and specificity, 36-100% and 64-100% respectively (Boelaert M., El-Safi S., Hailu A., Mukhtar M., Rijal S., Sundar S., Wasunna M., Aseffa A., Mbui J., Menten J., Desjeux P., Peeling R. W., Diagnostic tests for kala-azar: a multi-centre study of the freeze-dried DAT, rK39 strip test and KAtex in East Africa and the Indian subcontinent, Trans. R. Soc. Trop. Med. Hyg., 102(1):32-40 (2008); Salam M. A., Khan M. G., Mondal D., Urine antigen detection by latex agglutination test for diagnosis and assessment of initial cure of visceral leishmaniasis, Trans. R. Soc. Trop. Med. Hyg., 105(5):269-72 (2011)). In contrast, the capture ELISA reported here overcomes these drawbacks (particularly the specificity problems) because it detects proteins instead of carbohydrates. As it is well known, carbohydrates are very heterogeneous molecules particularly compared to the unique parasite proteins reported here. Moreover, the KATEX™ latex agglutination assay readout is based on degree of latex agglutination on a glass slide. This is of major concern due to the high subjectivity of this procedure.

### Overcoming Problems

Larger groups of VL caused by L. infantum (VL patients from the New World) and from VL caused by Leishmania donovani (VL patients from the Old World) will be tested. We expect that our combined capture ELISA test will be equally useful for the diagnosis of VL patients from the Old World. This prediction is based on the fact that the three antigens discovered in the urines of patients with VL from the New World are 98% homologous to the same group of proteins produced by L. donovani.

In addition, in order to increase the sensitivity of the capture ELISA we are in the process of preparing monoclonal antibodies against these antigens as well as large amounts of recombinant antigens for affinity purification of the polyclonal antibodies. These reagents should increase the sensitivity of the assay. Once this is achieved, we will convert the current ELISA format to a point of care device utilizing the same pair of antibodies.

### CONCLUSION

We introduced a promising non-invasive antigen detection assay for the diagnosis of VL. This new assay is based on three L. infantum proteins previously identified in the urine of patients with VL. These antigens were used to generate antibodies that were used in combination to assemble a single capture ELISA for the clinical detection of these antigens in the urine of patients with VL. This new urine based assay identified 20/20 VL patients and did not react with 62 urine samples obtained from control subjects. These encouraging results warrant a clinical trial to validate this important assay for the diagnosis of active VL. This trial will involve a large number of confirmed VL cases (including post-treated kala-azar), endemic controls, non-endemic controls, and other febrile diseases particularly in areas where these infections are co-endemic with VL.

The phrase “consists essentially of” or “consisting essentially of” refers to elements in the claimed invention that are essential or needed for the claimed invention to work or operate in any embodiment described herein. For example, with respect to diagnostic assays and kits, detection molecules and reagents for detecting the polypeptides described herein can be included. With respect to vaccine or pharmaceutical compositions, adjuvants or immune enhances, in certain embodiments, may be needed for the vaccine or composition to be effective.

The relevant teachings of all the references, patents and/or patent applications cited herein are incorporated herein by reference in their entirety.

While this invention has been particularly shown and described with references to preferred embodiments thereof, it will be understood by those skilled in the art that various changes in form and details can be made therein without departing from the scope of the invention encompassed by the appended claims.

What is claimed is:

1. An isolated polypeptide molecule comprising an immunogenic portion of an L. infantum or L. donovani antigen, wherein said antigen comprises an amino acid sequence selected from the group consisting of:
a) an amino acid sequence encoded by a nucleic acid having greater than or equal to about 70% identity with SEQ ID NO: 1, 3, 5, 7, 9, 11, 13, 15, 17, 19, 21, or combination thereof;
b) an amino acid sequence encoded by a nucleic acid molecule having greater than or equal to about 70% identity with coding region of SEQ ID NO: 1, 3, 5, 7, 9, 11, 13, 15, 17, 19, 21, or combination thereof;
c) an amino acid sequence encoded by a nucleic acid having greater than or equal to about 70% identity with a complement of SEQ ID NO: 1, 3, 5, 7, 9, 11, 13, 15, 17, 19, or 21; by coding region of a complement of SEQ ID NO: 1, 3, 5, 7, 9, 11, 13, 15, 17, 19, or 21; or combination thereof;
d) an amino acid sequence encoded by a nucleic acid molecule having greater than or equal to about 70% identity with a molecule that hybridizes to SEQ ID NO: 1, 3, 5, 7, 9, 11, 13, 15, 17, 19, or 21; to coding region of SEQ ID NO: 1, 3, 5, 7, 9, 11, 13, 15, 17, 19, or 21; to complement of SEQ ID NO: 1, 3, 5, 7, 9, 11, 13, 15, 17, 19, or 21, to coding region of a complement of SEQ ID NO: 1, 3, 5, 7, 9, 11, 13, 15, 17, 19, or 21, or to combination thereof; and
e) an amino acid sequence having greater than or equal to about 70% similarity to a sequence set forth in SEQ ID NO: 2, 4, 6, 8, 10, 12, 14, 16, 18, 20, 22, or combination thereof.

2. The isolated polypeptide molecule of claim 1, wherein the isolated polypeptide molecule stimulates an immunogenic specific visceral leishmaniasis (VL) response in a host.

3. An isolated nucleic acid molecule that encodes a polypeptide molecule that comprises an immunogenic portion of a L. infantum or L. donovani antigen, wherein said antigen is encoded by a nucleic acid molecule having greater than or equal to about 70% identity with a sequence selected from the group consisting of:
a) SEQ ID NO: 1, 3, 5, 7, 9, 11, 13, 15, 17, 19, 21, or combination thereof;
b) the coding region of SEQ ID NO: 1, 3, 5, 7, 9, 11, 13, 15, 17, 19, 21, or combination thereof;
c) a complement of SEQ ID NO: 1, 3, 5, 7, 9, 11, 13, 15, 17, 19, or 21; the coding region of a complement of SEQ ID NO: 1, 3, 5, 7, 9, 11, 13, 15, 17, 19, or 21; or combination thereof;
d) a sequence that hybridizes to SEQ ID NO: 1, 3, 5, 7, 9, 11, 13, 15, 17, 19, or 21; to the coding region of SEQ ID NO: 1, 3, 5, 7, 9, 11, 13, 15, 17, 19, or 21; to a complement of SEQ ID NO: 1, 3, 5, 7, 9, 11, 13, 15, 17, 19, or 21; to the coding region of a complement of SEQ ID NO: 1, 3, 5, 7, 9, 11, 13, 15, 17, 19, or 21; or combination thereof; and
e) a sequence that encodes SEQ ID NO: 2, 4, 6, 8, 10, 12, 14, 16, 18, 20, 22, or combination thereof.

4. The isolated nucleic acid molecule of claim 3, wherein the isolated nucleic acid molecule encodes a polypeptide molecule that stimulates an immunogenic specific VL response in a host.

5. The isolated nucleic acid molecule of claim 3, wherein the nucleic acid molecule is an RNA molecule.

6. A vector or plasmid that comprises the nucleic acid molecule of claim 3.

7. A host cell transformed with the nucleic acid sequence of claim 3.

8. An antibody that binds to a polypeptide of claim 1.

9. A fusion protein comprising a polypeptide selected from the group consisting of:
a) at least one of the polypeptides of claim 1;
b) at least two of the polypeptides of claim 1;
c) at least one polypeptide of claim 1 and a known L. infantum or L. donovani antigen; and
d) at least one polypeptide of claim 1 and an L. infantum or L. donovani antigen presented on a MHC Class-2 molecule.

10. A method for stimulating a specific immunogenic VL response in an individual, or preventing or reducing the severity of VL disease, the method comprises administering an amount of one or more of the polypeptide molecules of claim 1.

11. The method of claim 10, wherein the polypeptide molecule or the nucleic acid molecule is administered in a carrier.

12. A method for monitoring treatment of the VL disease in an individual, the method comprises:
a) detecting the level of one or more of the polypeptide molecules of claim 1 in a sample from the individual; and
b) comparing the level of the one or more molecules with a standard;
wherein a level of molecules that is higher than the standard indicates ineffective treatment, and a level that is less than or equal to the standard indicates effective treatment.

13. A method for monitoring treatment of the VL disease in an individual, the method comprises:
a) detecting the level of one or more polypeptide molecules of claim 1 in a sample from the individual at more than one time points; and
b) comparing the level of the one or more polypeptide molecules at the one or more time points, wherein an increase in the level of the molecules indicates ineffective treatment, and decrease or no change in the level indicates effective treatment.

14. A method of diagnosing VL disease in an individual, the method comprises detecting the presence or absence of one or more of the polypeptide molecules of claim 1; wherein the presence of the one or more polypeptide molecules indicates the presence of the VL disease and the absence of the one or more polypeptide molecules indicates the absence of the disease.

15. A method for detecting L. infantum or L. donovani infection in a biological sample, the method comprises assessing the presence of one or more of the polypeptide molecules of claim 1 in the sample, wherein the presence of the molecule indicates the presence of L. infantum or L. donovani infection; and the absence of the molecule indicates the absence of L. infantum or L. donovani infection.

16. A method for detecting L. infantum or L. donovani infection in a biological sample, the method comprises:
a) contacting the sample with an antibody that binds with a polypeptide molecule of claim 1, sufficiently to allow formation of a complex between the sample and the antibody, to thereby form an antigen-antibody complex; and
b) detecting the antigen-antibody complex, wherein the presence of the complex indicates the presence of L. infantum or L. donovani infection; and the absence of a complex indicates the absence of L. infantum or L. donovani infection.

17. A method for detecting L. infantum or L. donovani infection in a biological sample, the method comprises:
a) contacting the sample with at least two oligonucleotide primers in a polymerase chain reaction, wherein at least one of the oligonucleotide primers is specific for one or more of the isolated nucleic acid molecule of claim 3, sufficiently to allow amplification of the primers; and
b) detecting in the sample the amplified nucleic acid sequence; wherein the presence the amplified nucleic acid sequence indicates L. infantum or L. donovani infection, and the absence of the amplified nucleic acid sequence indicates an absence of L. infantum or L. donovani infection.

18. A method for detecting L. infantum or L. donovani infection in a biological sample, the method comprises:
a) contacting the sample with one or more oligonucleotide probes specific for the nucleic acid molecule of claim 3 under high stringency conditions, sufficiently to allow hybridization between the sample and the probe; and
b) detecting the nucleic acid molecule that hybridizes to the oligonucleotide probe in the sample; wherein the presence of hybridization of the probe indicates L. infantum or L. donovani infection, and the absence of hybridization indicates an absence of L. infantum or L. donovani infection.

19. The method of claim 18, wherein said antibody is detectably labeled.

20. The method of claim 19, wherein the method further includes contacting the sample with a second antibody specific to said antigen or said antigen-antibody complex.

21. The method of claim 20, wherein the polypeptide or the antibody is bound to a solid support.

22. The method of claim 18, wherein the biological sample is urine.

23. A composition that comprises the polypeptide sequence of claim 1 and a physiologically acceptable carrier.

24. The composition of claim 23, further including an immune response enhancer.

25. The composition of claim 24, wherein the immune response enhancer is an adjuvant or another VL antigen.

26. The composition of claim 25, wherein the composition is a vaccine composition.

27. The composition of claim 23, wherein the adjuvant includes at least one component selected from the group consisting of MPL and QS21.

28. The composition of claim 20, wherein the composition is formulated in an oil-in-water emulsion.

29. A kit for diagnosing the presence or absence of L. infantum or L. donovani infection in a person, wherein the kit comprises one or more reagents for detecting the polypeptide molecule of claim 1.

30. A kit for diagnosing the presence or absence of L. infantum or L. donovani infection in a person, wherein the kit comprises one or more reagents for detecting one or more nucleic acid molecules having a sequence of SEQ ID NO:1, 3, 5, 7, 9, 11, 13, 15, 17, 19, 21, or combination thereof; the complements of said sequences, and nucleic acid sequences that hybridize to a sequence recited in SEQ ID NO:1, 3, 5, 7, 9, 11, 13, 15, 17, 19, 21, or combination; and a detection reagent.

31. A method for detecting L. infantum or L. donovani infection in a biological sample, the method comprises:
a) contacting the sample with a combination of two or more antibodies that bind with two or more polypeptide molecules of claim 1, sufficiently to allow formation of a complex between the sample and one or more antibody of the combination, to thereby form an antigen-antibody complex; and
b) detecting the antigen-antibody complex;
wherein the presence of the complex indicates the presence of L. infantum or L. donovani infection; and the absence of a complex indicates the absence of L. infantum or L. donovani infection.

32. The method of claim 31, wherein the combination of two or more antibodies comprises antibodies raised against polypeptides comprising SEQ ID NOs: 18, 20, and 22.
